SEQUENCE LISTING (36 sequences)

SEQ ID NO: 1 - DNA, 2220 bp, Homo sapiens
atgccctctccgcgtccagtgctgcttagaggtgctcgcgccgctctgctgctgctgctgccgccccggctcttagcccgaccctcgctcctgctccgccggtccctcagcgcggcctcctgccccccgatctccttgcccgccgccgcctcccggagcagcatggacggcgcgggggctgaggaggtgctggcacctctgaggctagcagtgcgccagcagggagatcttgtgcgaaaactcaaagaagataaagcaccccaagtagacgtagacaaagcagtggctgagctcaaagcccgcaagagggttctggaagcaaaggagctggcgttacagcccaaagatgatattgtagaccgagcaaaaatggaagataccctgaagaggaggtttttctatgatcaagcttttgctatttatggaggtgttagtggtctgtatgactttgggccagttggctgtgctttgaagaacaatattattcagacctggaggcagcactttatccaagaggaacagatcctggagatcgattgcaccatgctcacccctgagccagttttaaagacctctggccatgtagacaaatttgctgacttcatggtgaaagacgtaaaaaatggagaatgttttcgtgctgaccatctattaaaagctcatttacagaaattgatgtctgataagaagtgttctgtcgaaaagaaatcagaaatggaaagtgttttggcccagcttgataactatggacagcaagaacttgcggatctttttgtgaactataatgtaaaatctcccattactggaaatgatctatcccctccagtgtcttttaacttaatgttcaagactttcattgggcctggaggaaacatgcctgggtacttgagaccagaaactgcacaggggattttcttgaatttcaaacgacttttggagttcaaccaaggaaagttgccttttgctgctgcccagattggaaattcttttagaaatgagatctcccctcgatctggactgatcagagtcagagaattcacaatggcagaaattgagcactttgtagatcccagtgagaaagaccaccccaagttccagaatgtggcagaccttcacctttatttgtattcagcaaaagcccaggtcagcggacagtccgctcggaaaatgcgcctgggagatgctgttgaacagggtgtgattaataacacagtattaggctatttcattggccgcatctacctctacctcacgaaggttggaatatctccagataaactccgcttccggcagcacatggagaatgagatggcccattatgcctgtgactgttgggatgcagaatccaaaacatcctacggttggattgagattgttggatgtgctgatcgttcctgttatgacctctcctgtcatgcacgagccaccaaagtcccacttgtagctgagaaacctctgaaagaacccaaaacagtcaatgttgttcagtttgaacccagtaagggagcaattggtaaggcatataagaaggatgcaaaactggtgatggagtatcttgccatttgtgatgagtgctacattacagaaatggagatgctgctgaatgagaaaggggaattcacaattgaaactgaagggaaaacatttcagttaacaaaagacatgatcaatgtgaagagattccagaaaacactatatgtggaagaagttgttccgaatgtaattgaaccttccttcggcctgggtaggatcatgtatacggtatttgaacatacattccatgtacgagaaggagatgaacagagaacattcttcagtttccctgctgtagttgctccattcaaatgttccgtcctcccactgagccaaaaccaggagttcatgccatttgtcaaggaattatcggaagccctgaccaggcatggagtatctcacaaagtagacgattcctctgggtcaatcggaaggcgctatgccaggactgatgagattggcgtggcttttggtgtcaccattgactttgacacagtgaacaagaccccccacactgcaactctgagggaccgtgactcaatgcggcagataagagcagagatctctgagctgcccagcatagtccaagacctagccaatggcaacatcacatgggctgatgtggaggccaggtatcctctgtttgaagggcaagagactggtaaaaaagagacaatcgaggaatga

SEQ ID NO: 2 - protein, 685 aa, Homo sapiens
MDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 3 - DNA, 21 bp, Artificial Sequence
  Synthetic oligonucleotide
gcatggagtatctcacaaagt

SEQ ID NO: 4 - DNA, 21 bp, Artificial Sequence
  Synthetic oligonucleotide
gcccaaagatgatattgtaga

SEQ ID NO: 5 - DNA, 21 bp, Artificial Sequence
  Synthetic oligonucleotide
gctgcccagattggaaattct

SEQ ID NO: 6 - DNA, 19 bp, Artificial Sequence
  Synthetic oligonucleotide
ggcgttacagcccaaagat

SEQ ID NO: 7 - DNA, 19 bp, Artificial Sequence
  Synthetic oligonucleotide
gcccaaagatgatattgta

SEQ ID NO: 8 - DNA, 19 bp, Artificial Sequence
  Synthetic oligonucleotide
cctggaggcagcactttat

SEQ ID NO: 9 - DNA, 19 bp, Artificial Sequence
  Synthetic oligonucleotide
gctgcccagattggaaatt

SEQ ID NO: 10 - DNA, 19 bp, Artificial Sequence
  Synthetic oligonucleotide
ggagcaattggtaaggcat

SEQ ID NO: 11 - DNA, 19 bp, Artificial Sequence
  Synthetic oligonucleotide
gcaattggtaaggcatata

SEQ ID NO: 12 - DNA, 19 bp, Artificial Sequence
  Synthetic oligonucleotide
ccgaatgtaattgaacctt

SEQ ID NO: 13 - DNA, 19 bp, Artificial Sequence
  Synthetic oligonucleotide
gcctgggtaggatcatgta

SEQ ID NO: 14 - DNA, 19 bp, Artificial Sequence
  Synthetic oligonucleotide
ggagatgaacagagaacat

SEQ ID NO: 15 - DNA, 19 bp, Artificial Sequence
  Synthetic oligonucleotide
gcatggagtatctcacaaa

SEQ ID NO: 16 - DNA, 19 bp, Artificial Sequence
  Synthetic oligonucleotide
ccagaatgtggcagacctt

SEQ ID NO: 17 - DNA, 19 bp, Artificial Sequence
  Synthetic oligonucleotide
cctgggtaggatcatgtat

SEQ ID NO: 18 - DNA, 19 bp, Artificial Sequence
  Synthetic oligonucleotide
ggcccagcttgataactat

SEQ ID NO: 19 - DNA, 19 bp, Artificial Sequence
  Synthetic oligonucleotide
gggtacttgagaccagaaa

SEQ ID NO: 20 - DNA, 19 bp, Artificial Sequence
  Synthetic oligonucleotide
ggcagaaattgagcacttt

SEQ ID NO: 21 - DNA, 19 bp, Artificial Sequence
  Synthetic oligonucleotide
ggaagaagttgttccgaat

SEQ ID NO: 22 - DNA, 19 bp, Artificial Sequence
  Synthetic oligonucleotide
ggatcatgtatacggtatt

SEQ ID NO: 23 - DNA, 19 bp, Artificial Sequence
  Synthetic oligonucleotide
ggcatggagtatctcacaa

SEQ ID NO: 24 - DNA, 19 bp, Artificial Sequence
  Synthetic oligonucleotide
cctatgctttgaaggttct

SEQ ID NO: 25 - DNA, 19 bp, Artificial Sequence
  Synthetic oligonucleotide
gctttgaaggttctcgtgt

SEQ ID NO: 26 - DNA, 19 bp, Artificial Sequence
  Synthetic oligonucleotide
tcagagctgtgtccctgaa

SEQ ID NO: 27 - DNA, 19 bp, Artificial Sequence
  Synthetic oligonucleotide
gcaaatctgttcgctcgca

SEQ ID NO: 28 - DNA, 19 bp, Artificial Sequence
  Synthetic oligonucleotide
gcggcgatttcatcatgct

SEQ ID NO: 29 - DNA, 19 bp, Artificial Sequence
  Synthetic oligonucleotide
gcgatttcatcatgctccg

SEQ ID NO: 30 - DNA, 19 bp, Artificial Sequence
  Synthetic oligonucleotide
cccaaagatgatattgtag

SEQ ID NO: 31 - DNA, 19 bp, Artificial Sequence
  Synthetic oligonucleotide
gctgtgctttgaagaacaa

SEQ ID NO: 32 - DNA, 19 bp, Artificial Sequence
  Synthetic oligonucleotide
gcactttatccaagaggaa

SEQ ID NO: 33 - DNA, 19 bp, Artificial Sequence
  Synthetic oligonucleotide
tcccattactggaaatgat

SEQ ID NO: 34 - DNA, 19 bp, Artificial Sequence
  Synthetic oligonucleotide
gctatttcattggccgcat

SEQ ID NO: 35 - DNA, 19 bp, Artificial Sequence
  Synthetic oligonucleotide
gcatctacctctacctcac

SEQ ID NO: 36 - DNA, 21 bp, Artificial Sequence
  Synthetic oligonucleotide
ggcatagggacccatcattga

(19) United States
(12) Patent Application Publication
(10) Pub. No.: US 2019/0381086 A1
(43) Pub. Date: Dec. 19, 2019

(54) INHIBITION OF NEDDYLATION USING GLYCYL-tRNA SYNTHETASE INHIBITORS

(71) Applicant: The Scripps Research Institute, La Jolla, CA (US)

(72) Inventors: Xiang-Lei Yang, San Diego, CA (US); Zhongying Mo, San Diego, CA (US); Paul Schimmel, Hobe Sound, FL (US)

(21) Appl. No.: 15/767,627

(22) PCT Filed: Oct. 13, 2016
(86) PCT No.: PCT/US16/56862

§ 371 Date: Apr. 11, 2018

Related U.S. Application Data
(60) Provisional application No. 62/241,386, filed on Oct. 14, 2015.

(51) Int. Cl. A61K 31/7076; A61K 31/713; C12N 15/113

(52) U.S. Cl. CPC A61K 31/7076; C12N 2310/122; C12N 15/113; A61K 31/713

(57) ABSTRACT

Disclosed herein are methods and compositions for inhibiting neddylation using Glycyl-tRNA synthase (GlyRS) inhibitors. Also disclosed are related compositions and methods for treating diseases such as cancer.

### CROSS-REFERENCE TO RELATED APPLICATIONS

The present application is a U.S. national phase application under 35 U.S.C. § 371 of International Application No. PCT/US2016/056862, filed on Oct. 13, 2016, and published on Apr. 20, 2017; which claims the benefit of priority to U.S. Provisional Patent Application No. 62/241,386, filed Oct. 14, 2015. The content of each of these related applications is incorporated by reference herein in its entirety.

### STATEMENT REGARDING FEDERALLY SPONSORED R&D

This invention was made with government support under National Institutes of Health grant R01GM088278. The U.S. Government has certain rights in this invention.

### REFERENCE TO SEQUENCE LISTING

The present application is being filed along with a Sequence Listing in electronic format. The Sequence Listing is provided as a file entitled SEQLISTING.TXT, created Oct. 7, 2015, which is 15.2 Kb in size. The information in the electronic format of the Sequence Listing is incorporated herein by reference in its entirety.

### BACKGROUND

### Field of the Disclosure

The present disclosure relates to the fields of molecular biology and medicine. In particular, disclosed herein are compositions and methods for inhibiting neddylation using Glycyl-tRNA synthase (GlyRS) inhibitors, and related compositions and methods for treating diseases such as cancer.

### Description of the Related Art

The NEDD8 pathway plays a critical role in the activation of the ubiquitin E3 ligase activity of cullin-RING ligase (CRL) E3s via the covalent attachment of NEDD8 to the core cullin protein of these enzyme complexes. This process of neddylation has been shown to be essential for the E3 ligase activity of CRLs. CRLs are a large superfamily of E3s that are responsible for the ubiquitination of multiple substrate proteins, including several that are involved in the regulation of normal cellular function as well as some that have been shown to be associated with cancer.

### SUMMARY

Some embodiments disclosed herein relate to methods of reducing neddylation in a cell. In some embodiments, the methods comprise: contacting a cell with a composition comprising a Glycyl-tRNA synthetase (GlyRS) inhibitor, wherein the level of neddylation is decreased in the cell. Some embodiments relate to methods of reducing neddylation in a cell population. In some embodiments, the methods comprise: acquiring knowledge of the level of neddylation in a cell population; and contacting the cell population with a composition comprising a Glycyl-tRNA synthetase (GlyRS) inhibitor to thereby decrease the level of neddylation in the cell population. Some embodiments relate to methods of reducing neddylation in a cell population. In some embodiments, the methods comprise: identifying a cell population having undesirable level of neddylation; and contacting the cell population with a composition comprising a Glycyl-tRNA synthetase (GlyRS) inhibitor to thereby decrease the level of neddylation in the cell population.

In the methods of reducing neddylation, the composition can be, for example, a pharmaceutical composition. In some embodiments, the GlyRS inhibitor inhibits GlyRS functions in aminoacylation and neddylation. In some embodiments, the GlyRS inhibitor does not significantly inhibit GlyRS function in aminoacylation.

In some embodiments, the GlyRS inhibitor is an inhibitor for a mammalian GlyRS protein. In some embodiments, the GlyRS inhibitor is an inhibitor for a human GlyRS protein. In some embodiments, the human GlyRS protein comprises an amino acid sequence having at least 90% identity to the amino acid sequence set forth in SEQ ID NO: 2. In some embodiments, the GlyRS inhibitor is an inhibitor for a plant GlyRS protein.

The GlyRS inhibitor can be, for example, a protein, a nucleic acid, a small molecule compound, or a combination thereof. In some embodiments, the GlyRS inhibitor is an RNA molecule capable of silencing the expression of a GlyRS gene. In some embodiments, the GlyRS inhibitor is an RNA molecule that binds to an mRNA encoded by a GlyRS gene. In some embodiments, the GlyRS gene is a mammalian GlyRS gene, for example a human GARS gene. In some embodiments, the human GARS gene consists of or comprises a nucleotide sequence having at least 90% sequence identity to the nucleotide sequence set forth in SEQ ID NO: 1. In some embodiments, the GlyRS inhibitor is a short-hairpin RNA (shRNA) consisting of or comprising a nucleotide sequence having at least 90% sequence identity to the nucleic acid sequence of SEQ ID NOs: 3-35. For example, the GlyRS inhibitor is a RNA consisting of or comprising the nucleic acid sequence of SEQ ID NOs:3-35. In some embodiments, the GlyRS inhibitor is GlySA or a derivative thereof.

In some embodiments, the GlyRS inhibitor interferes with the binding between the GlyRS protein and NEDD8 in the cell or the cell population. In some embodiments, the GlyRS inhibitor interferes with the binding between the GlyRS protein and Ubc12 in the cell or the cell population. In some embodiments, the GlyRS inhibitor interferes with the binding between the GlyRS protein and NEDD8-conjugated Ubc12 in the cell or the cell population. In some embodiments, the GlyRS inhibitor binds to one or more of amino acids 84-93 of SEQ ID NO: 2. In some embodiments, the GlyRS inhibitor binds to one or more of amino acids 232-238 of SEQ ID NO: 2. In some embodiments, the GlyRS inhibitor binds to one or more of amino acids Arg277, Glu279, Val289, Glu296, Ile404, and Glu552 of SEQ ID NO: 2. In some embodiments, the GlyRS inhibitor is a GlySA derivative. In some embodiments, the GlyRS inhibitor decreases the amount of NEDD8-conjugated Ubc12 in the cell or the cell population. In some embodiments, the GlyRS inhibitor decreases Ubc12 activity in the cell or the cell population. In some embodiments, the GlyRS inhibitor increases Ubc12 degradation in the cell or the cell population. In some embodiments, the GlyRS inhibitor interferes with the binding between a GlyRS protein and heterodimeric E1 enzyme for neddylation (APPBP1/UBA3) in the cell or the cell population. In some embodiments, the GlyRS inhibitor decreases neddylation of a cullin protein in the cell or the cell population.

In some embodiments, the cell is a mammalian cell. In some embodiments, the contacting is performed in vitro, ex vivo, or in vivo. In some embodiments, the cell or the cell population is present in a tissue or in a body of a subject. In some embodiments, the level of neddylation in the cell or the cell population is reduced by at least 50%.

Some embodiments disclosed herein relate to isolated double-stranded ribonucleic acid (dsRNA) molecules that inhibit expression of a Glycyl-tRNA synthetase (GlyRS) gene, wherein a first strand of the dsRNA is substantially identical to at least 19 consecutive nucleotides of the GlyRS gene, and a second strand of the dsRNA is substantially complementary to the first strand. In some embodiments, the GlyRS gene is a mammalian GlyRS gene, for example a human GARS gene. In some embodiments, the human GARS gene comprises or consists of a nucleotide sequence having at least 90% identity to the nucleotide sequence set forth in SEQ ID NO: 1. In some embodiments, the GlyRS gene is a plant GlyRS gene. In some embodiments, the dsRNA is encoded by a polynucleotide, wherein the first strand and the second strand of the dsRNA are transcribed from said polynucleotide and form a hairpin loop.

Some embodiments disclosed herein relate to isolated single stranded oligonucleotides that are complementary to a portion of a Glycyl-tRNA synthetase (GlyRS) gene of at least 10 consecutive nucleotides. In some embodiments, the GlyRS gene is a mammalian GlyRS gene, for example a human GARS gene. In some embodiments, the human GARS gene comprises or consists of a nucleotide sequence having at least 90% identity to the nucleotide sequence set forth in SEQ ID NO: 1. In some embodiments, the GlyRS gene is a plant GlyRS gene.

Some embodiments disclosed herein relate to methods of identifying an inhibitor of neddylation. The methods, in some embodiments, comprise: providing a test compound; testing the testcompound for its ability to reduce or inhibit the binding between a Glycyl-tRNA synthetase (GlyRS) protein and NEDD8-conjugated Ubc12; and identifying the compound as an inhibitor of neddylation if the compound has the ability to reduce or inhibit the binding between the GlyRS protein and NEDD8-conjugated Ubc12. In some embodiments, the inhibitors bind to the catalytic domain of the GlyRS protein. The GlyRS protein can be a mammalian GlyRS protein, for example a human GlyRS protein. In some embodiments, the human GlyRS protein comprises or consists of an amino acid sequence having at least 90% sequence identity to the amino acid sequence set forth in SEQ ID NO:2. In some embodiments, the methods comprise testing the test compound for its ability to reduce or inhibit the aminoacylation activity of the GlyRS protein. In some embodiments, the methods comprise testing one or more additional test compounds for their ability to reduce or inhibit the binding between the GlyRS protein and NEDD8-conjugated Ubc12. In some embodiments, the methods comprise testing the one or more additional test compounds for their ability to reduce or inhibit the aminoacylation activity of the GlyRS protein.

Also disclosed herein are compositions comprising a Glycyl-tRNA synthetase (GlyRS) inhibitor, for example pharmaceutical compositions comprising one or more pharmaceutically acceptable excipients. In some embodiments, the GlyRS inhibitor is an isolated siRNA molecule that binds to an mRNA of the GlyRS protein. In some embodiments, the GlyRS inhibitor is a molecule that inhibits binding between the GlyRS protein and NEDD8-conjugated Ubc12. In some embodiments, the GlyRS inhibitor is GlySA or a derivative thereof. In some embodiments, the GlyRS inhibitor inhibits GlyRS functions in aminoacylation and neddylation. In some embodiments, the GlyRS inhibitor does not significantly inhibit GlyRS function in aminoacylation.

Some embodiments disclosed herein relate to methods of reducing cell proliferation. In some embodiments, the methods comprise: contacting a cell with a composition comprising a Glycyl-tRNA synthetase (GlyRS) inhibitor, whereby the proliferation of the cell is reduced. In some embodiments, the activity of the CRL1 (cullin1-RING) ubiquitin ligases is inhibited in the cell. In some embodiments, the activity of a substrate of the CRL1 ubiquitin ligase is increased in the cell. In some embodiments, the substrate of the CRL1 ubiquitin ligase is selected from the group consisting of c-Myc, c-Jun, cyclin E, Emil, Cdt-1, pIκBα, NRF2, HIF-1α, β-catenin, Cdc25A, mTOR, BimEL and p27. In some embodiments, the methods comprise providing MLN4924 to the cell. The cell can be, for example, a mammalian cell, a plant cell. In some embodiments, the proliferation of the cell is reduced by at least 50%. In some embodiments, the GlyRS inhibitor inhibits GlyRS functions in aminoacylation and neddylation. In some embodiments, the GlyRS inhibitor does not significantly inhibit GlyRS function in aminoacylation.

Also disclosed herein are methods of treating or ameliorating cancer in a subject. In some embodiments, the methods comprise: administering a therapeutically effective amount of a pharmaceutical composition comprising a Glycyl-tRNA synthetase (GlyRS) inhibitor to a subject in need thereof. In some embodiments, the pharmaceutical composition further comprises one or more of additional therapeutic agents. In some embodiments, the methods comprise administering one or more additional pharmaceutical compositions comprising one or more of additional therapeutic agents. In some embodiments, the cancer is breast cancer, ovarian cancer, lung cancer, breast duct carcinoma, colorectal adenocarcinoma and lung squamous cell carcinoma, or a combination thereof. In some embodiments, the cancer is selected from the group consisting of breast cancer, cervical cancer, colon cancer, liver cancer, prostate cancer, melanoma, ovarian cancer, lung cancer, renal cell carcinoma, Schwannoma, mesothelioma, acute myeloid leukemia, multiple myeloma, non-Hodgkin lymphoma, and a combination thereof. In some embodiments, the cancer is a solid tumor. In some embodiments, the cancer is a hematological malignancy. In some embodiments, the GlyRS inhibitor is GlySA or a GlySA derivative. In some embodiments, the GlyRS inhibitor inhibits GlyRS functions in aminoacylation and neddylation. In some embodiments, the GlyRS inhibitor does not significantly inhibit GlyRS function in aminoacylation. In some embodiments, the GlyRS inhibitor is an inhibitor for a human GlyRS

### BRIEF DESCRIPTION OF THE DRAWINGS

FIGS. 1A-E show specific binding between GlyRS and NEDD8. FIG. 1A: A schematic flowchart of the neddylation pathway. FIG. 1B: The domain composition of human GlyRS. FIG. 1C: NEDD8, but not ubiquitin and SUMO1, specifically binds to His-tagged GlyRS, but not His-tagged SerRS and TrpRS. FIG. 1D: GlyRS binds to NEDD8 in vivo. HEK293 cells were transfected with Myc-NEDD8 constructs. 48 hours after transfection cells were harvested and lysed with acid lysis buffer and used for immunoprecipitation assay. FIG. 1E: Domain mapping by His-Tag pull-down assay suggests that NEDD8 binds to catalytic domain of GlyRS.

FIGS. 2A-D shows that GlyRS plays critical role in neddylation. FIG. 2A: Over-expression of human GlyRS specifically leads to increase of Ubc12^{N8} in HEK293 cells. The cells were transfected with pcDNA6V5 vectors containing the indicated gene fragment with a V5-tag. FIGS. 2B-D: Ubc12^{N8} and substrates neddylation decrease specifically upon GlyRS knock-down. Knockdown of the expression of GlyRS, but not SerRS, specifically decreases the levels of Ubc12^{N8}, and NEDD8-modified cullin proteins in HeLa cells. HeLa cells were transfected by pLenti vectors containing either a scramble sequence, SerRS or GlyRS specific sequences respectively. Cells were harvested 48 hours after transfection and lysed with acid lysis buffer and subjected to SDS-PAGE.

FIGS. 3A-H show that GlyRS preferentially binds to and promote Ubc12^{N8}. FIG. 3A: Co-immunoprecipitation indicates that GlyRS prefers binding to Ubc12^{N8} than to the apo Ubc12. V5-tagged GlyRS and Flag-tagged Ubc12-C111S genes were co-transfected to HEK293 cell for the assay. FIG. 3B: Strong binding of Ubc12^{N8} to GlyRS as determined by biolayer interferometry. A.U. indicates Arbitrary Unit. Binding analysis to immobilized GST-GlyRS was carried out with the same concentration (62.5 nM) of NEDD8, Ubc12, or Ubc12^{N8} at 30°. FIG. 3C: Biolayer interferometry analysis confirms that GlyRS binding to Ubc12^{N8}. Binding analysis to immobilized Ubc12^{N8} was carried out with a range of concentrations of full-length (8.07 nM-129 nM) GlyRS. FIG. 3D: Structural model of the GlyRS-Ubc12^{N8} interaction generated using Patchdock (a server for molecular docking). FIGS. 3E-G: Biolayer interferometry analysis confirms that F84-L93 and I232-M238 regions, but not Insertion 1, are important for GlyRS binding to Ubc12^{N8}. Binding analysis to immobilized Ubc12^{N8} was carried out with a range of concentrations (1.25-20 μg/mL) of ΔF84-L93 (8.16-130 nM), ΔI232-M238 (8.13-130 nM), and Alnsertion 1 (62.5-125 nM) GlyRS. FIG. 3H: Over-expression of ΔI232-M238 GlyRS in HEK293 cells cannot promote NEDD8 conjugation of Ubc12. HEK293 cells were transfected with pcDNA6V5 vectors containing the indicated gene fragment with a V5-tag.

FIGS. 4A-E show that GlyRS captures Ubc12^{N8} and escorts it to substrates. FIG. 4A: GlyRS binds to APPBP1 in HEK293 cells. Cells were lysed with lysis buffer and used for immunoprecipitation assay. FIG. 4B: Structural model for how GlyRS protects Ubc12^{N8} during the neddylation cascade. The interaction between GlyRS and E1 (APPBP1 subunit) is modeled by using the Patchdock server. The interaction between E1^{N8} and Ubc12 is adapted from a crystal structure (PDB 2NVU). The crystal structure of Ubc12^{N8} is from PDB 4P50. The transient interaction between E1 and Ubc12^{N8} is modeled by aligning the NEDD8 molecule from the above two crystal structures. FIG. 4C: Synergistic effect between GlyRS and Ubc12^{N8} for interacting with E1 suggesting the capture of Ubc12^{N8} by GlyRS as the conjugated E2 is released from E1. Binding analysis to immobilized E1 was carried out with 5 μg/mL of Ubc12^{N8} (119 nM), 5 μg/mL of GlyRS (32 nM), or the mixture of Ubc12^{N8} and GlyRS each at 5 μg/mL. The black dotted line indicates the calculated sum of the binding curves for Ubc12^{N8} and GlyRS to E1. FIG. 4D: The ABD domain alone lacks the synergistic effect with Ubc12^{N8} for binding to E1. Binding analysis to immobilized E1 was carried out with 5 μg/mL of Ubc12^{N8} (119 nM), 5 μg/mL of ABD GlyRS (289 nM), or the mixture of Ubc12^{N8} and ABD each at 5 μg/mL. The black dotted line indicates the calculated sum of the binding curves for Ubc12^{N8} and ABD to E1, separately. FIG. 4E: Cullin1 competes off GlyRS for Ubc12^{N8} interaction. Binding analysis to immobilized Ubc12^{N8} was carried out with 20 μg/mL of GlyRS (128 nM), 20 μg/mL of cullin1_{cfd}/Rbx1 (385 nM), or the mixture of cullin1_{cfd}/Rbx1 and GlyRS each at 20 μg/mL. The black dotted line indicates the calculated sum of the binding curves for cullin1_{ctd}/Rbx1 and GlyRS to Ubc12^{N8}.

FIGS. 5A-C show that GlyRS is involved in cell cycle regulation via neddylation. FIG. 5A: A schematic figure showing how cell cycle kinase inhibitor p27^{kip} half-life is tightly regulated for proper cell cycle progression. p27^{kip} undergoes fast turn over through neddylation activated poly-ubiquitination directed degradation and NEDD8 specific inhibitor MLN4924 could disrupt this and result in abnormal accumulation of p27^{kip}. FIG. 5B: Knock-down of GARS extends p27^{kip} half-life. As indicated, HeLa cells were transfected by pLenti vectors containing either a scramble sequence, or GlyRS specific sequences respectively. 24 hours after transfection, cells were treated with fresh medium containing either 0.204 MLN4924 or DMSO for 24 hours. Cell medium were then replaced with that containing cycloheximide (30 μg/mL) at indicated time. Meanwhile some cells were treated with 20 μM MG132 and harvested after 7 hours. Cells were harvested and lysed using acid lysis buffer and then subjected to SDS-PAGE. FIG. 5C: FACS analyses of cell cycle confirmed GlyRS involved in cell cycle regulation. Briefly, HeLa cells were transfected with indicated constructs and 24 hours after transfection cells were treated with either 0.2 μM MLN4924 or DMSO for another 24 hours. Cells were then collected, fixed and stained with PI and analyzed by flow cytometry. Cells treated with MLN4924 or GARS knock-down showed significant drop of the 2N peak and sequestered in the 4N population, indicating cell cycle arrest.

FIG. 6 shows that GlyRS binds to NEDD8 via its catalytic domain. Hydrogen-deuterium exchange (HDX) analysis shows that NEDD8 bind mainly to the catalytic domain of GlyRS. Changes in deuterium incorporation resulting from the GlyRS-NEDD8 interaction are mapped to the protein sequence and the crystal structure of GlyRS (PDB 2PME).

FIGS. 7A-B shows that GlyRS knockdown does not affect Ube2F conjugation. FIG. 7A: Ube2F^{N8} remains unchanged upon GlyRS knockdown. HeLa cells were transfected by pLenti vectors containing either a scramble sequence, SerRS or GlyRS specific sequences respectively. Cells were harvested 48 hours after transfection and lysed with acid lysis buffer and subjected to SDS-PAGE. FIG. 7B: Ube2F binds to GlyRS much weaker compared to that of Ubc12. Biolayer interferometry analysis confirms that Ube2F does not bind to GlyRS compared to that of Ubc12. Binding analysis to immobilized GST-GlyRS was carried out with 1.0 μM of either Ubc12 or Ube2F.

FIGS. 8A-C show that GlyRS catalytic domain mediates interaction with Ubc12. FIG. 8A: Domain mapping by GST pull-down assay suggests that Ubc12 also binds to the catalytic domain of GlyRS. FIG. 8B: Hydrogen-deuterium exchange (HDX) analysis confirms that Ubc12 binds to the catalytic domain of GlyRS. Changes in deuterium incorporation resulting from the GlyRS-Ubc12 interaction are mapped to the protein sequence and the crystal structure of GlyRS (PDB 2PME). FIG. 8C: GlyRS but not BSA significantly extends the half-life of Ubc12^{N8} in vitro. Ubc12^{N8} (504) were incubated with GlyRS, BSA or same volume of PBS buffer at 37° for indicated time in the PBS buffer (PH7.4 supplemented with 5 mM DTT). Samples were then subjected to SDS-PAGE and stained with commassie blue. The images of the gels were then quantified by ImageJ and plotted against the time. Error bars represent standard deviations for the image quantification (n=3).

FIGS. 9A-F show that GlyRS binds to APPBP1 and facilitates cullin neddylation. FIG. 9A: Domain mapping by GST pull-down suggests that E1 (APPBP1/UBA3) binds to the anti-codon binding domain (ABD) of GlyRS, as ABD alone can be pulled-down by GST-APPBP1. FIG. 9B: Biolayer interferometry analysis confirms that ABD alone is sufficient for E1 interaction, as full-length, AWHEP, and ABD GlyRS bind to E1 (APPBP1/UBA3) with similar affinity. Binding analysis to immobilized E1 was carried out with a range of concentrations (62.5-500 nM) of full-length, AWHEP, or ABD GlyRS. FIG. 9C: Biolayer interferometry analysis confirms that ABD alone binds to E1 regulatory subunit APPBP1, as ABD GlyRS binds to APPBP1 with similar affinity to that of APPBP1/UBA3. Binding analysis to immobilized APPBP1 was carried out with a range of concentrations (0.58-4.6 μM) of ABD GlyRS. FIG. 9D: Molecular docking of GlyRS (PDB 2PME) and APPBP1-UBA3 (PDB 2NVU) by using Patchdock. FIG. 9E: Modeling analysis suggesting that GlyRS is unlikely to interfere with NEDD8 transferring from Ubc12 to cullin. The complex structure of cullin1-Rbx1-Ubc12^{N8} is adapted from PDB 4P50. Thioester bond formed between NEDD8 and Ubc12 is exposed outside while bound to GlyRS and would not interfere with its transfer to cullin. FIG. 9F: In vitro neddylation assay shows GlyRS facilitate cullin neddylation.

FIG. 10 shows a non-limiting schematic illustration showing that GlyRS is associated with cell proliferation via dual cellular functions. The schematic summarizes the dual function of GlyRS in aminoacylation as an enzyme and in neddylation as a chaperone that supports protein synthesis and cell-cycle progression.

FIG. 11 shows bioinformatic data demonstrating that high level of GlyRS is associated with rapid breast cancer progression. The expression of all cytoplamic human tRNA synthetases in breast cancer was analyzed by Kaplan-Meier plots and hazard ratio (HR). Patient samples were divided in halves as low-expression and high-expression sets for each tRNA synthetase in the analysis. n=3557 patients. P values were calculated with two-sided log-rank tests.

FIG. 12 shows bioinformatic data demonstrating that high level of GlyRS is associated with rapid ovarian cancer progression. Kaplan-Meier plots and hazard ratio (HR) of the expression of human GlyRS in stage 2 ovarian cancer were analyzed. Patient samples were divided in halves as low-expression and high-expression sets for GlyRS in the analysis. n=60 patients. P values were calculated with two-sided log-rank tests.

FIG. 13 shows bioinformatic data demonstrating that high level of GlyRS is associated with rapid lung cancer progression. Kaplan-Meier plots and hazard ratio (HR) of the expression of human GlyRS in lung squamous cell carcinoma were analyzed. Patient samples were divided in halves as low-expression and high-expression sets for GlyRS in the analysis. n=524 patients. P values were calculated with two-sided log-rank tests.

FIGS. 14A-B show higher level of GlyRS staining in most malignant patient cancer tissue samples. FIG. 14A shows that high level staining of GlyRS is observed in patient tissue samples of breast duct carcinoma, colorectal adenocarcinoma and lung squamous cell carcinoma. FIG. 14B shows that most malignant patient cancer tissue samples have higher level of GlyRS expression compared to normal tissue.

FIG. 15 is a non-limiting schematic illustration of GlySA binding to GlyRS active site (PDB 2ZT8). GlySA is an analog of Gly-AMP, reaction intermediate of GlyRS.

FIG. 16 is a plot showing that GlySA (but not MLN4924) inhibits GlyRS aminoacylation. The aminoacylation assay was performed using recombinant human GlyRS (200 nM) proteins at room temperature. MLN4924 is an inhibitor of neddylation currently used in clinical trials for multiple solid and hematopoietic cancers. MLN4924 targets the E1 enzyme (UBA3) of neddylation.

FIG. 17 shows experimental data demonstrating that GlySA decreases GlyRS binding to activated NEDD8 E2 (Ubc12^{N8}). The interactions of GlyRS (0.25 μM) to that of Ubc12^{N8} (100 nM; immobilized to the sensor tips) were compared in the presence of DMSO or GlySA at 30° C. by biolayer interferometry (Octet).

FIG. 18 shows experimental data demonstrating that unlike MLN4924, GlySA does not affect NEDD8 E1 (UBA3) activation. In vitro NEDD8 activation assay was performed with recombinant human APPBP1-UBA3 (2.7 μM) protein and fluorescein-labeled NEDD8 proteins at 37° C. for 1 hour. The concentration of GlySA and MLN4924 was 300 μM.

FIG. 19 shows experimental data demonstrating that GlySA, but not SerSA, TyrSA, inhibits cullin neddylation in MDA-MB-231 cells. MDA-MB-231 cells at 80% confluence were treated overnight with each compound and then the cells were harvested and lysed with the acid lysis buffer and subjected to non-reducing SDS-PAGE. SerSA and TyrSA are analogs of Ser-AMP and Tyr-AMP, reaction intermediate of SerRS and TyrRS, respectively. MLN4924 was used as a positive control for the experiment.

FIG. 20 shows experimental data determining IC₅₀ of GlySA for inhibiting cullin neddylation in MDA-MB-231 cells. MDA-MB-231 cells at 80% confluence were treated overnight with GlySA and then the cells were harvested and lysed with the acid lysis buffer and subjected to non-reducing SDS-PAGE.

FIG. 21 shows experimental data on time course of GlySA in inhibiting neddylation in MDA-MB-231 cells. MDA-MB-231 cells at 80% confluence were treated with 200 nM GlySA and then the cells were harvested at different time points and lysed with the acid lysis buffer and subjected to non-reducing SDS-PAGE.

FIG. 22 shows experimental data on GlySA effect over a range of concentrations on key components and substrates of the neddylation pathway in MDA-MB-231 cells. MDA-MB-231 cells at 80% confluence were treated overnight with GlySA and then the cells were harvested and lysed with the acid lysis buffer and subjected to non-reducing SDS-PAGE.

FIG. 23 shows experimental data on GlySA effect over a range of concentrations on key components and substrates of neddylation in MDA-MB-468 cells. MDA-MB-468 cells at 80% confluence were treated overnight with GlySA and then the cells were harvested and lysed with the acid lysis buffer and subjected to non-reducing SDS-PAGE.

FIG. 24 shows experimental data on GlySA effect over a range of concentrations on key components and substrates of neddylation in MCF7 cells. MCF7 cells at 80% confluence were treated overnight with GlySA and then the cells were harvested and lysed with the acid lysis buffer and subjected to non-reducing SDS-PAGE.

FIG. 25 shows a schematic illustration of a non-limiting exemplary maximum tolerant dosage assay of GlySA. GlySA (DMSO stock solution diluted by saline) were administrated to three month old female BALB CJ mice via tail vein injections. Mice after four injections were evaluated and all were alive. The GlySA concentration tested were 0.4 mg/kg (10 μM), 2.0 mg/kg (50 μM), 4.0 mg/kg (100 μM). N=3 for each group.

FIG. 26 shows a schematic illustration of non-limiting exemplary lung metastasis assay methods. 1×10⁵ MDA-MB-231 cells were injected via tail vein to NOD.Cg-Prkdc^{scid} Il2rg mice. Then mice were separated into 3 groups. Group A: vehicle alone (PBS with 1% DMSO), group B: GlySA (4 mg/kg; 100 μM), group C: MLN4924 GlySA (4.4 mg/kg; 100 μM) were administrated via tail vein injections twice per week. N=10 for each group.

FIG. 27 shows experimental data demonstrating that GlySA treatment reduces lung metastasis in mice. Top panel: mice lungs 14 days after tumor cells (MDA-MB-231) injection. White dots show the surface tumor colonies. Bottom panel: numbers of lung metastasis colonies are analyzed by two tails unpaired T test. The error bars represent SEM (n=8-10).

FIG. 28 shows a non-limiting schematic illustration of a working model of GlySA on inhibiting both protein synthesis and cell-cycle regulation.

FIG. 29 shows GlySA and several GlySA derivatives. The top panel shows chemical structures of GlySA and several GlySA derivatives. The bottom panel shows the key interacting residues on GlyRS with GlySA based on a co-crystal structure of GlySA bound GlyRS (PDB: 2ZT8).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### General Techniques

The practice of the techniques described herein may employ, unless otherwise indicated, conventional techniques and descriptions of organic chemistry, polymer technology, molecular biology (including recombinant techniques), cell biology, biochemistry, sequencing technology, and micro- and nano-fabrication which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, Biochemistry (4th Ed.) (1995) W.H. Freeman, New York N.Y.; Gait, Oligonucleotide Synthesis: A Practical Approach (2002) IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W.H. Freeman Pub., New York, N.Y., Jaeger, Introduction to Microelectronic Fabrication (2002) 2nd Ed., Prentice Hall, and Madou, Fundamentals of Microfabrication (2002) all of which are herein incorporated in their entireties by reference for all purposes.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, N.Y. 1994). All publications mentioned herein are incorporated by reference for the purpose of describing and disclosing devices, formulations and methodologies that may be used in connection with the presently described methods and disclosures.

For purposes of the present disclosure, the following terms are defined below.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

The terms “polypeptide”, “oligopeptide”, “peptide,” and “protein” are used interchangeably herein to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may include, for example, modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The terms “polynucleotide,” “oligonucleotide,” “nucleic acid” and “nucleic acid molecule” are used interchangeably herein to refer to a polymeric form of nucleotides of any length, e.g., at least 8, 9, 10, 20, 30, 40, 50, 100, 200, 300, 400, 500, 1,000 or more nucleotides, and may include ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g., nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

As used herein, “sequence identity” or “identity” or “homology” in the context of two protein sequences (or nucleotide sequences) includes reference to the residues in the two sequences which are the same when aligned for maximum correspondence over a specified comparison window. The portion of the amino acid sequence or nucleotide sequence in the comparison window may comprise additions or deletions (i.e., gaps) as compared to the reference sequence for optimal alignment of the two sequences. When percentage of sequence identity is used in reference to proteins it is recognized that residue positions which are not identical often differ by conservative amino acid substitutions, where amino acids are substituted for other amino acid residues with similar chemical properties (e.g. charge or hydrophobicity) and therefore do not change the functional properties of the molecule. Where sequences differ in conservative substitutions, the percentage sequence identity may be adjusted upwards to correct for the conservative nature of the substitutions. Sequences, which differ by such conservative substitutions are said to have “sequence similarity” or “similarity”. Means for making these adjustments are well known to persons skilled in the art. The percentage is calculated by determining the number of positions at which the identical amino acid or nucleic acid base residue occurs in both sequences to yield the number of matched positions, dividing the number of matched positions by the total number of positions in the window of comparison and multiplying the result by 100 to yield the percentage of sequence identity. Typically this involves scoring a conservative substitution as a partial rather than a full mismatch, thereby increasing the percentage sequence identity. Thus, for example, where an identical amino acid is given a score of 1 and a non-conservative substitution is give a score of zero, a conservative substitution is given a score between 0 and 1. The scoring of conservative substitutions is calculated, e.g. according to the algorithm of Meyers and Miller (Computer Applic. Biol. Sci., 1998, 4, 11-17).

As used herein, the term “homologue” is used to refer to a nucleic acid which differs from a naturally occurring nucleic acid (i.e., the “prototype” or “wild-type” nucleic acid) by minor modifications to the naturally occurring nucleic acid, but which maintains the basic nucleotide structure of the naturally occurring form. Such changes include, but are not limited to: changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art for the production of nucleic acids including, but not limited to, recombinant DNA techniques, chemical synthesis, or any combination thereof.

As used herein, “complementary or matched” means that two nucleic acid sequences have at least 50% sequence identity. For example, the two nucleic acid sequences can have at least 60%, 70%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. For example, the two nucleic acid sequences can have at least 95%, 96%, 97%, 98%, 99% or 100% of sequence identity. Alternatively, “substantially complementary or substantially matched” means that two nucleic acid sequences can hybridize under high stringency condition(s).

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e. Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place.

As used herein, “administration” or “administering” refers to a method of giving a dosage of a pharmaceutically active ingredient to a vertebrate.

As used herein, a “dosage” refers to an amount of therapeutic agent administered to a patient.

As used herein, a “daily dosage” refers to the total amount of therapeutic agent administered to a patient in a day.

As used herein, the term “therapeutic agent” means a substance that is effective in the treatment of a disease or condition.

As used herein, “therapeutically effective amount” or “pharmaceutically effective amount” is meant an amount of therapeutic agent, which has a therapeutic effect. The dosages of a pharmaceutically active ingredient which are useful in treatment are therapeutically effective amounts. Thus, as used herein, a therapeutically effective amount refers to an amount of therapeutic agent which produces the desired therapeutic effect as judged by clinical trial results and/or model animal studies.

As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “treatment” refers to a clinical intervention made in response to a disease, disorder or physiological condition manifested by a patient, particularly a patient suffering from cancer. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing the symptoms. This takes place at primary, secondary and tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

A therapeutic agent or a protective agent may comprise a “drug.” As used herein, a “drug” refers to a therapeutic agent or a diagnostic agent and includes any substance, other than food, used in the prevention, diagnosis, alleviation, treatment, or cure of a disease. Stedman's Medical Dictionary, 25th Edition (1990). The drug can include any substance disclosed in at least one of: The Merck Index, 12th Edition (1996); Pei-Show Juo, Concise Dictionary of Biomedicine and Molecular Biology, (1996); U.S. Pharmacopeia Dictionary, 2000 Edition; and Physician's Desk Reference, 2001 Edition. In some embodiments, the therapeutic agent is one of the embodiments of the compositions described herein.

In some embodiments, the drug used in the therapeutic system will often be placed on, embedded, encapsulated or otherwise incorporated into a delivery matrix. The delivery matrix may be included in or on either the first skeletal structure or the second cushioning structure, or both. The delivery matrix, in turn, comprises either a biodegradable or a non-biodegradable material. The delivery matrix may include, although it is not limited to, a polymer. Examples of biodegradable polymers include protein, hydrogel, polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid), and combinations thereof. Non-biodegradable polymers may comprise silicone, acrylates, polyethylenes, polyurethane, polyurethane, hydrogel, polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, polymer foam, silicone rubber, polyethylene terephthalate, ultra-high molecular weight polyethylene, polycarbonate urethane, polyurethane, polyimides, stainless steel, nickel-titanium alloy (e.g., Nitinol), titanium, stainless steel, cobalt-chrome alloy (e.g., ELGILOY® from Elgin Specialty Metals, Elgin, Ill.; CONICHROME® from Carpenter Metals Corp., Wyomissing, Pa.). In one embodiment, the hydrogel may comprise poly(alkyleneoxides), such as poly(ethyleneoxide), also known as polyethyleneglycols or PEGs.

The term “comprising” as used herein is synonymous with “including,” “containing,” or “characterized by,” and is inclusive or open-ended and does not exclude additional, unrecited elements or method steps.

Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present disclosure will become apparent from the following specification taken in conjunction with the accompanying drawings.

In the following description, numerous specific details are set forth to provide a more thorough understanding of the present disclosure. However, it will be apparent to one of skill in the art that the methods of the present disclosure may be practiced without one or more of these specific details. In other instances, well-known features and procedures well known to those skilled in the art have not been described in order to avoid obscuring the disclosure.

### GlyRS Proteins and Polynucleotides

Glycyl-tRNA synthetase (GlyRS; also known as glycine-tRNA ligase) is an enzyme that belongs to the aminoacyl tRNA synthetase (aaRS) family. aaRS is an enzyme that attaches the appropriate amino acid onto its tRNA. It does so by catalyzing the esterification of a specific cognate amino acid or its precursor to one of all its compatible cognate tRNAs to form an aminoacyl-tRNA. GlyRS is an enzyme that catalyzes the chemical reaction:

ATP+glycine+tRNAGly [symbol] AMP+diphosphate+glycyl-tRNAGly

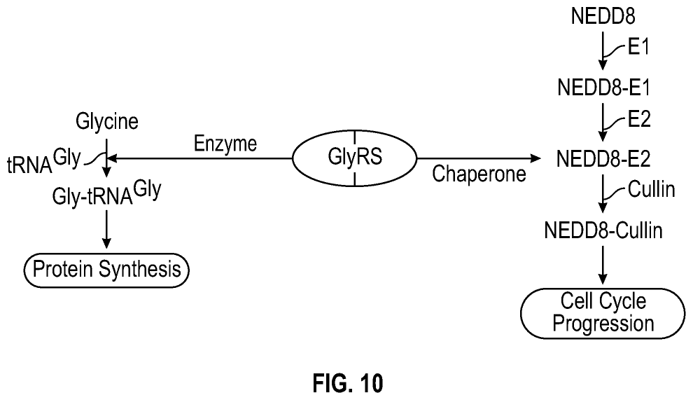

The three substrates of the GlyRS enzyme are ATP, glycine, and tRNA(Gly), whereas the three products are AMP, diphosphate and glycyl-tRNA(Gly). Human GlyRS is encoded by the GARS gene, and is composed of three distinct domains: the N-terminal metazoan-specific WHEP domain, catalytic domain, and the C-terminal anticodon-binding domain (ABD). As described herein (for example shown in FIG. 10), a GlyRS, in some embodiments, can function in aminoacylation as an enzyme and in neddylation (for example as a chaperon that supports protein synthesis and cell-cycle progress).

In some embodiments, the GlyRS proteins disclosed herein are capable of interaction with one or more components of the neddylation pathway, including NEDD8, E1 and E2. For example, the GlyRS proteins may capable of binding to the APPBP1 subunit of E1 and activated E2 (NEDD8-conjugated Ubc12). In some embodiments, the GlyRS proteins are capable of increasing the level of neddylation in a cell, for example, neddylation of E1, E2, and neddylation substrates. Neddylation substrates include, but are not limited to, members of the cullin protein family, e.g., cullin 1, cullin 2, cullin 3, cullin 4A, cullin 4B, cullin 5, cullin 7, and cullin 9. In some embodiments, the substrates are human cullin proteins. In some embodiments, the GlyRS proteins are capable of increasing the level of cullin-RING ubiquitin ligases (CRLs) in a cell. Without being bound by any particular theory, it is believed that the GlyRS proteins disclosed herein may increase the level of neddylation through interacting with NEDD8-conjugation Ubc12 and protecting it from degradation. In some embodiments, the GlyRS proteins do not interact with other ubiquitin or ubiquitin-like proteins such as SUMO1.

The coding sequence of a human GARS gene is shown below (SEQ ID NO: 1). Also contemplated herein are GlyRS nucleotide sequences that have at least 70%, at least 80%, at least 85%, at least 90%, at least 95%, at least 98%, at least 99%, or more sequence identity to SEQ ID NO:1.

| | | | | | | |
|---|---|---|---|---|---|---|
| 361 | ccctctccgc | gtccagtgct | gcttagaggt | gctcgcgccg | ctctgctgct | gctgctgccg |
| 421 | ccccggctct | tagcccgacc | ctcgctcctg | ctccgccggt | ccctcagcgc | ggcctcctgc |
| 481 | cccccgatct | ccttgcccgc | cgccgcctcc | cggagcagca | tggacggcgc | gggggctgag |
| 541 | gaggtgctgg | cacctctgag | gctagcagtg | cgccagcagg | gagatcttgt | gcgaaaactc |
| 601 | aaagaagata | aagcacccca | agtagacgta | gacaaagcag | tggctgagct | caaagcccgc |
| 661 | aagagggttc | tggaagcaaa | ggagctggcg | ttacagccca | aagatgatat | tgtagaccga |
| 721 | gcaaaaatgg | aagataccct | gaagaggagg | tttttctatg | atcaagcttt | tgctatttat |
| 781 | ggaggtgtta | gtggtctgta | tgactttggg | ccagttggct | gtgctttgaa | gaacaatatt |
| 841 | attcagacct | ggaggcagca | ctttatccaa | gaggaacaga | tcctggagat | cgattgcacc |
| 901 | atgctcaccc | ctgagccagt | tttaaagacc | tctggccatg | tagacaaatt | tgctgacttc |
| 961 | atggtgaaag | acgtaaaaaa | tggagaatgt | tttcgtgctg | accatctatt | aaaagctcat |
| 1021 | ttacagaaat | tgatgtctga | taagaagtgt | tctgtcgaaa | agaaatcaga | aatggaaagt |
| 1081 | gttttggccc | agcttgataa | ctatggacag | caagaacttg | cggatctttt | tgtgaactat |
| 1141 | aatgtaaaat | ctcccattac | tggaaatgat | ctatcccctc | cagtgtcttt | taacttaatg |
| 1201 | ttcaagactt | tcattgggcc | tggaggaaac | atgcctgggt | acttgagacc | agaaactgca |
| 1261 | caggggattt | tcttgaattt | caaacgactt | ttggagttca | accaaggaaa | gttgcctttt |
| 1321 | gctgctgccc | agattggaaa | ttcttttaga | aatgagatct | cccctcgatc | tggactgatc |
| 1381 | agagtcagag | aattcacaat | ggcagaaatt | gagcactttg | tagatcccag | tgagaaagac |
| 1441 | caccccaagt | tccagaatgt | ggcagacctt | cacctttatt | tgtattcagc | aaaagcccag |
| 1501 | gtcagcggac | agtccgctcg | gaaaatgcgc | ctgggagatg | ctgttgaaca | gggtgtgatt |
| 1561 | aataacacag | tattaggcta | tttcattggc | cgcatctacc | tctacctcac | gaaggttgga |
| 1621 | atatctccag | ataaactccg | cttccggcag | cacatggaga | atgagatggc | ccattatgcc |
| 1681 | tgtgactgtt | gggatgcaga | atccaaaaca | tcctacggtt | ggattgagat | tgttggatgt |
| 1741 | gctgatcgtt | cctgttatga | cctctcctgt | catgcacgag | ccaccaaagt | cccacttgta |
| 1801 | gctgagaaac | ctctgaaaga | acccaaaaca | gtcaatgttg | ttcagtttga | acccagtaag |
| 1861 | ggagcaattg | gtaaggcata | taagaaggat | gcaaaactgg | tgatggagta | tcttgccatt |
| 1921 | tgtgatgagt | gctacattac | agaaatggag | atgctgctga | atgagaaagg | ggaattcaca |
| 1981 | attgaaactg | aagggaaaac | atttcagtta | acaaaagaca | tgatcaatgt | gaagagattc |
| 2041 | cagaaaacac | tatatgtgga | agaagttgtt | ccgaatgtaa | ttgaaccttc | cttcggcctg |
| 2101 | ggtaggatca | tgtatacggt | atttgaacat | acattccatg | tacgagaagg | agatgaacag |
| 2161 | agaacattct | tcagtttccc | tgctgtagtt | gctccattca | aatgttccgt | cctcccactg |
| 2221 | agccaaaacc | aggagttcat | gccatttgtc | aaggaattat | cggaagccct | gaccaggcat |
| 2281 | ggagtatctc | acaaagtaga | cgattcctct | gggtcaatcg | gaaggcgcta | tgccaggact |
| 2341 | gatgagattg | gcgtggcttt | tggtgtcacc | attgactttg | acacagtgaa | caagaccccc |
| 2401 | cacactgcaa | ctctgaggga | ccgtgactca | atgcggcaga | taagagcaga | gatctctgag |
| 2461 | ctgcccagca | tagtccaaga | cctagccaat | ggcaacatca | catgggctga | tgtggaggcc |
| 2521 | aggtatcctc | tgtttgaagg | gcaagagact | ggtaaaaaag | agacaatcga | ggaatgaatg |

The amino acid sequence of a human GlyRS is shown below (SEQ ID NO: 2). Also contemplated herein are GlyRS proteins having sequences that have at least 70%, at least 80%, at least 85%, at least 90%, at least 95%, at least 98%, at least 99%, or more sequence identity to SEQ ID NO: 2.

MDGAGAEEVLAPLRLAVRQQGDLVRKLKEDKAPQVDVDKAVAELKARK
RVLEAKELALQPKDDIVDRAKMEDTLKRRFFYDQAFAIYGGVSGLYDF
GPVGCALKNNIIQTWRQHFIQEEQILEIDCTMLTPEPVLKTSGHVDKF
ADFMVKDVKNGECFRADHLLKAHLQKLMSDKKCSVEKKSEMESVLAQL
DNYGQQELADLFVNYNVKSPITGNDLSPPVSFNLMFKTFIGPGGNMPG
YLRPETAQGIFLNFKRLLEFNQGKLPFAAAQIGNSFRNEISPRSGLIR
VREFTMAEIEHFVDPSEKDHPKFQNVADLHLYLYSAKAQVSGQSARKM
RLGDAVEQGVINNTVLGYFIGRIYLYLTKVGISPDKLRFRQHMENEMA
HYACDCWDAESKTSYGWIEIVGCADRSCYDLSCHARATKVPLVAEKPL
KEPKTVNVVQFEPSKGAIGKAYKKDAKLVMEYLAICDECYITEMEMLL
NEKGEFTIETEGKTFQLTKDMINVKRFQKTLYVEEVVPNVIEPSFGLG
RIMYTVFEHTFHVREGDEQRTFFSFPAVVAPFKCSVLPLSQNQEFMPF
VKELSEALTRHGVSHKVDDSSGSIGRRYARTDEIGVAFGVTIDFDTVN
KTPHTATLRDRDSMRQIRAEISELPSIVQDLANGNITWADVEARYPLF
EGQETGKKETIEE

GlyRS proteins suitable for the embodiments of the present disclosure may be produced with recombinant DNA technology in various host cells. For example, expression vectors capable of expressing eukaryotic proteins (e.g., plasmid pcDNA6) may be used to express the recombinant GlyRS proteins. In some embodiments, the host cells can be bacterial, fungal, plant, yeast, insect or mammalian cells. The term host cell includes both the cells, progeny of the cells and protoplasts created from the cells that are used to produce a GlyRS according to the disclosure. In some embodiments, the host cells are prokaryotic cells, for example bacteria host cells.

As a non-limiting example, to produce the GlyRS protein with the recombinant DNA technology, a DNA construct comprising nucleic acid encoding the amino acid sequence of the designated GlyRS can be constructed and transferred into, for example, an E. coli host cell. The vector may be any vector which when introduced into an E. coli host cell can be integrated into the host cell genome and can be replicated. The nucleic acid encoding the GlyRS can be operably linked to a suitable promoter, which shows transcriptional activity in E. coli host cell. The promoter may be derived from genes encoding proteins either homologous or heterologous to the host cell. As used herein, an “inducible promoter” may refer to a promoter that is active under environmental or developmental regulation.

In some embodiments, the GlyRS coding sequence can be operably linked to a signal sequence. In some embodiments, the expression vector may also include a termination sequence. In one embodiment, the termination sequence and the promoter sequence can be derived from the same source. In another embodiment, the termination sequence can be homologous to the host cell.

In some embodiments, the expression vector may include one or more selectable markers. Examples of representative selectable markers include ones that confer antimicrobial resistance (e.g., hygromycin and phleomycin). In some embodiments, nutritional selective markers including those markers known in the art as amdS, argB, and pyr4, can be used as the selectable marker.

An expression vector comprising a DNA construct with a polynucleotide encoding the GlyRS may be any vector which is capable of replicating autonomously in a given host organism or of integrating into the DNA of the host. In some embodiments, the expression vector can be a plasmid or a viral construct.

In some embodiments, two types of expression vectors for obtaining expression of genes are contemplated. For example, the first expression vector may comprise DNA sequences in which the promoter, GlyRS-coding region, and terminator all originate from the gene to be expressed. In some embodiments, gene truncation can be obtained by deleting undesired DNA sequences (e.g., DNA encoding unwanted domains) to leave the domain to be expressed under control of its own transcriptional and translational regulatory sequences. The second type of expression vector may be preassembled and contains sequences needed for high-level transcription and a selectable marker. In some embodiments, the coding region for the GARS gene or part thereof can be inserted into this general-purpose expression vector such that it is under the transcriptional control of the expression construct promoter and terminator sequences. In some embodiments, genes or part thereof may be inserted downstream of a strong promoter.

Methods used to ligate the DNA construct comprising a polynucleotide encoding the GlyRS, a promoter, a terminator and other sequences and to insert them into a suitable vector are well known in the art. Linking can be generally accomplished by ligation at convenient restriction sites. If such sites do not exist, the synthetic oligonucleotide linkers are used in accordance with conventional practice (Bennett & Lasure, More Gene Manipulations In Fungi, Academic Press, San Diego (1991) pp 70-76). Additionally, vectors can be constructed using known recombination techniques (e.g., Invitrogen Life Technologies, Gateway Technology).

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; and protoplast fusion. General transformation techniques are known in the art (see, e.g., Campbell et al., (1989) Curr. Genet. 16:53-56).

In some embodiments, genetically stable transformants can be constructed with vector systems whereby the nucleic acid encoding GlyRS is stably integrated into a host strain chromosome. Transformants can then be purified by known techniques.

### Methods of Inhibiting Neddylation

NEDD8 is an 81-amino acid protein with 9 kDa relative molecular mass and is 60% identical and 80% homologous to ubiquitin. NEDD8 has a dedicated E1-activating enzyme (AppBp1/UBA3, or NAE) and E2-conjugating enzymes (UBC12, UBE2F) and is essential for the enzymatic activity of the CRL family of E3 ligases, through conjugation to the cullin scaffold. Other components of the neddylation pathway include DEN1 which processes NEDD8 to its mature, 76-amino acid form, and the COPS signalosome complex, which is responsible for removing NEDD8 from cullin proteins. CAND1 (cullin-associated and neddylation-dissociated) is an additional component that regulates CRL complex assembly by binding to the cullin in the absence of NEDD8 activation.

Neddylation is a posttranslational modification that controls cell cycle and proliferation by conjugating the ubiquitin-like protein NEDD8 to specific targets. It is hereby disclosed that GlyRS plays a critical role in neddylation. In human cells, knockdown of GlyRS, but not a different tRNA synthetase, decreases the global level of neddylation and delays cell cycle progression. This function of GlyRS is achieved through direct interactions with multiple components of the neddylation pathway, including NEDD8, E1, and E2. GlyRS can bind to the APPBP1 subunit of E1 to capture and protect the activated E2 (NEDD8-conjugated Ubc12) before it reaches a downstream target.

Some embodiments disclosed herein provide methods of reducing or inhibiting neddylation in a cell. As used herein, inhibition of neddylation includes partially or fully blocks or abolished neddylation in a cell or a cell population. For example, the inhibition can reduce 1%, 5%, 10%, 15%, 20%, 25%, 30%, 35%, 40%, 45%, 50%, 60%, 70%, 80%, 90%, 95%, 99%, or a range between any two of these values, of neddylation in the cell or the cell population. In some embodiments, the inhibition can reduce about 1%, 5%, 10%, 15%, 20%, 25%, 30%, 35%, 40%, 45%, 50%, 60%, 70%, 80%, 90%, 95%, 99%, or a range between any two of these values, of neddylation in the cell or the cell population. In some embodiments, the neddylation in the cell or the cell population is completely abolished. In some embodiments, the methods comprise contacting the cell with a GlyRS inhibitor, wherein the level of neddylation is decreased in the cell. For example, the level of neddylation can be decreased to, or to about, 99%, 95%, 90%, 80%, 70%, 60%, 50%, 45%, 40%, 35%, 30%, 25%, 20%, 15%, 10%, 5%, 1%, or a range between any two of these values, of the level of neddylation in cell(s) not treated with the GlyRS inhibitor. Neddylation, in some embodiments, refers to the conjugation of NEDD8 to components of the neddylation pathway, e.g., the E1 enzyme, the E2 enzyme, or the E3 ligases. In some embodiments, neddylation may refer to the conjugation of NEDD8 to a cullin protein in the E3 ligases, e.g., a CRL. The decrease of neddylation may occur to individual components of the neddylation pathway, or at a global level. In some embodiments, the level of neddylation may be reduced by 10%, 20%, 30%, 40%, 50%, 60%, 70%, 80%, 90%, 95%, 98%, 99%, or more.

It would be appreciated that the GlyRS inhibitor may decrease the level of neddylation in the cell through a variety of mechanisms, for example, by interfering the binding between the GlyRS protein and NEDD8, by interfering the binding between the GlyRS protein and an E2 enzyme, such as Ubc12, by interfering the binding between the GlyRS protein and NEDD8-conjugated Ubc12, by interfering the binding between the GlyRS protein and an E1 enzyme, such as APPBP1/UBA3 heterodimer, by directly targeting E1 enzyme (given the similarity in the first step reaction between GlyRS and E1), or any combination thereof. In some embodiments, the GlyRS inhibitor may inhibit the activity or decrease the level of a component of the neddylation pathway, for example, the level of NEDD8-conjugated Ubc12, the activity of the Ubc12 enzyme, the protein level of the Ubc12 enzyme, or any combination thereof. In some embodiments, the GlyRS inhibitor binds to both GlyRS and E1 enzyme to decrease neddylation.

It will also be appreciated by one of skill in the art that inhibiting neddylation by the methods disclosed herein may result in the inhibition of ubiquitination of one or more of the E3 substrates in a cell, such as ATF4, CCNE1, CDC25, CDKN1A, CDKN1B, CTNNB1, DAPK1, Emil, FancM, HIF2A, IRS1, JUN, MCL1, NRF2, ORC1L, PDCD4, POLR2A, SETD8, SNAI3, USP18, etc.

### GlyRS Inhibitors

As discussed above, the term “GlyRS inhibitor” is used herein in a broad sense and includes any molecule that partially or fully blocks, inhibits or neutralizes a biological activity mediated by GlyRS. In some embodiments, it can prevent the activation of GlyRS. The term “GlyRS inhibitor” also includes any molecule that abolishes or reduces the function or expression of GlyRS.

The method by which GlyRS is inhibited is not limited in any way. In some embodiments, the GlyRS inhibitor can act directly on GlyRS, for example by binding to GlyRS, to prevent or reduce activation of GlyRS. In some embodiments, the GlyRS inhibitor can interfere, preferably abolish or reduce, GlyRS from interacting with a binding partner or a substrate, such a component of the neddylation pathway. In some embodiments, the GlyRS inhibitor can modulate the level of GlyRS gene expression, for example, inhibiting or reducing the transcription of GlyRS gene. In some embodiments, the GlyRS inhibitor can modulate the levels of GlyRS protein in cells by, for example, inhibiting or reducing the translation of GlyRS mRNA, or increasing the degradation of GlyRS mRNA or GlyRS protein. In some embodiments, the GlyRS inhibitor can block the interaction of GlyRS with NEDD8 and/or NEDD8-conjugated Ubc12.

As disclosed herein, a GlyRS can perform function in various biological processes, for example aminoacylation and neddylation. As used herein, a compound is considered to be a GlyRS inhibitor if the compound can reduce or inhibit one or more biological activities of a GlyRS. For example, a GlyRS inhibitor may reduce or inhibit GlyRS functions in both aminoacylation and neddylation. In some embodiments, the GlyRS inhibitor only reduce or inhibit GlyRS function in neddylation. In some embodiments, the GlyRS inhibitor does not significantly inhibit GlyRS function in aminoacylation. For example, a GlyRS inhibitor does not significantly inhibit GlyRS function in aminoacylation if the GlyRS inhibitor can at most reduce the activity of the GlyRS function in aminoacylation by, or by about, 80%, 70%, 60%, 50%, 40%, 30%, 20%, 10%, 50%, 1%, or a range between any two of these values. In some embodiments, the GlyRS inhibitor reduces the activity of the GlyRS function in aminoacylation by, or by about, 90%, 80%, 70%, 60%, 50%, 40%, 30%, 20%, 10%, 50%, 1%, or a range between any two of these values.

The types of GlyRS inhibitor are not limited in any way. GlyRS inhibitors include, for example, small molecules, nucleic acids, antibodies, peptides, or any combination thereof. In some embodiments, the GlyRS inhibitor can be a small molecule that binds to GlyRS. In some embodiments, the GlyRS inhibitor can be a molecule that blocks interaction of GlyRS and it binding partner. In some embodiments, the GlyRS inhibitor is a nucleic acid, for example, an anti-GlyRS small-hairpin RNA (shRNA) or an GlyRS anti-sense RNA.

Some embodiments of the present disclosure therefore include, for example, inhibitors of GlyRS function, for example, its interactions with components of the neddylation pathway. The GlyRS inhibitors can be used, for example, in any of the methods described herein. Any agent that may prevent or reduce the interaction between the GlyRS protein and NEDD8, E1, and E2, or eliminate or reduce the level of GlyRS protein expression, is contemplated by the present disclosure. A reduction refers to at least 10%, 20%, 30%, 40%, 50%, 60%, 70%, 80%, 90%, 95%, 99% or more of the interaction between the GlyRS protein and NEDD8, E1, and E2, or of the GlyRS protein expression level in a cell. Interaction refers the binding of NEDD8, E1, and E2 with the GlyRS protein, which may lead to a conformational change to the GlyRS protein and/or the NEDD8, E1, and E2 protein.

The ability of a molecule to inhibit GlyRS activity can be measured using assays that are known in the art. For example and without limitation, GlyRS inhibitors can be identified using biolayer interferometry. Briefly, in biolayer interferometry, a molecule can be examined for the ability to affect the binding between a GlyRS protein and its binding partner. For example, GlyRS proteins are immobilized on either anti-GST or Ni-NTA sensor tips in 96-well plates. A binding partner, e.g., NEDD8, is added to the buffer. Candidate GlyRS molecules are added to the buffer and the dissociation constants K_{d} are measured. GlyRS inhibitors that interfere with the binding between the GlyRS protein and NEDD8 can be identified based on the change in the dissociation constants K_{d}. One of skill in the art will be able to select the appropriate assays and reaction conditions based on the particular circumstances.

Some embodiments disclosed herein provide small molecule compounds that inhibit the function of a GlyRS protein. Non-limiting examples of inhibitory small molecule compounds include ones that exhibit binding specificity for at least one region of the GlyRS protein that is involved in its interaction with a component of the neddylation pathway, and/or its stability/degradation in a cell.

As used herein, the term “small molecule” refers to an organic compound that is of synthetic or biological origin (biomolecule), but is typically not a polymer.

The small molecule compounds disclosed herein may bind to a region of the GlyRS protein involved in its interaction with a component of the neddylation pathway, e.g., the catalytic domain, the ABD domain, or a combination thereof. For example, the small molecule compounds may interfere with the interaction between GlyRS and NEDD8, E1, and/or E2. In some embodiments, the small molecule compounds bind to a sequence comprising amino acids 84-93 of SEQ ID NO: 2. In some embodiments, the small molecule compounds bind to one or more of amino acids 84-93 of SEQ ID NO: 2. In some embodiments, the small molecule compounds bind to a sequence comprising amino acids 232-238 of SEQ ID NO: 2. In some embodiments, the small molecule compounds bind to one or more of amino acids 232-238 of SEQ ID NO: 2.

One non-limiting example of the GlyRS inhibitor is glycylsulfamoyladenosine (Gly-SA). The structure of this compound is as follows.

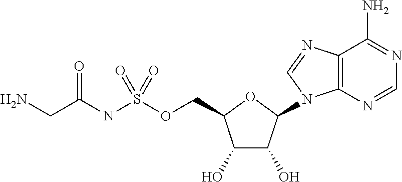 [SMILES: NCC(=O)NS(=O)(=O)OC[C@H]1O[C@@H](n2cnc3c(N)ncnc32)[C@H](O)[C@@H]1O]

Gly-SA is an analogue of the Gly-AMP reaction intermediate and inhibits GlyRS catalytic activity. Estimates of the potency of inhibition are obtained by performing enzyme assays in the presence of a range of inhibitor concentrations, and fitting the effect of inhibitor concentration on enzyme velocity to a four parameter logistic function that allows calculation of an IC₅₀ (the inhibitor concentration at which GlyRS activity is reduced by half). This parameter is directly related to the dissociation constant for inhibitor binding (Kj or K_{d}) and has a value of around 2.4 mM for Gly-SA when tested against the S. aureus GlyRS. Binding of Gly-SA to GlyRS can also be measured directly using stopped-flow fluorescence techniques because enzyme:inhibitor binary complex has around 5% higher tryptophan fluorescence than the free enzyme.

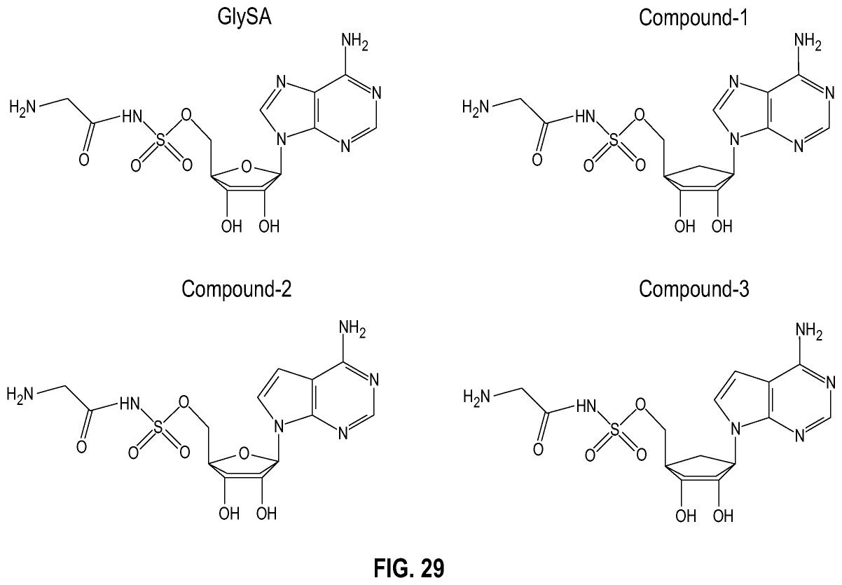

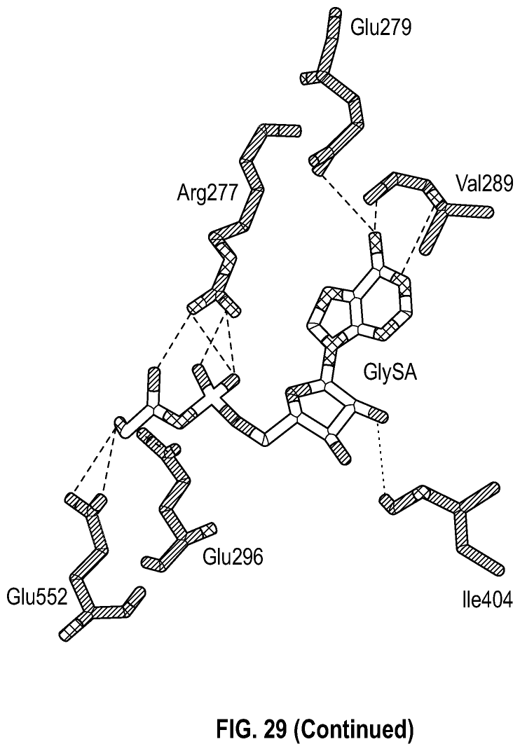

Also disclosed herein are Gly-SA derivatives that function as GlyRS inhibitors. Non-limiting examples of Gly-SA derivatives include Compound-1, Compound-2, and Compound-3 (shown below and in FIG. 29) and the analogues described for aaSA in Van de Vijver et al. (2008) J. Med. Chem. 51:3020-3029 (the content of which is incorporated by reference herein in its entirety). Moreover, chemical modifications for various aaRS inhibitors having similar chemical structure with Gly-SA have been described in, for example, Brown et al. (2000) Biochemistry 39(20):6003-6011, Lee et al. (2003) Bioorganic & Medicinal Chemistry Letters 12:1087-1092, Bernier et al. (2005) Bioorganic & Medicinal Chemistry 13:69-75, and Balg et al. (2007) Bioorganic & Medicinal Chemistry 15:295-304. One of skill in the art will appreciate that those chemical modifications can be introduced to Gly-SA in some embodiments to produce Gly-SA derivatives.

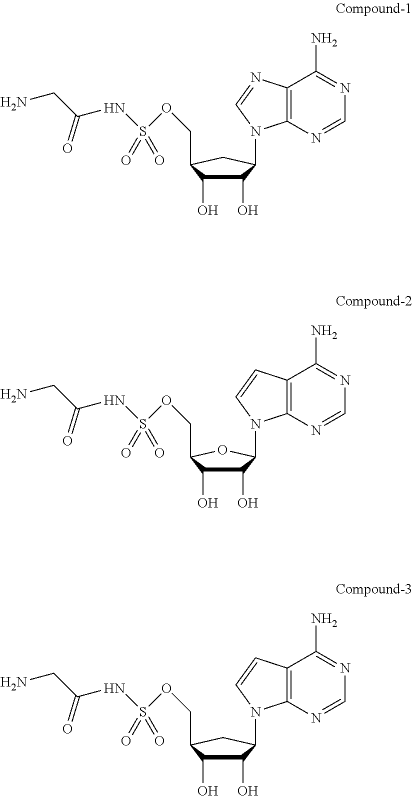

Also disclosed herein are siRNA and shRNA against GlyRS. Double-stranded RNA (dsRNA) directs the sequence-specific degradation of mRNA through a process known as RNA interference (RNAi). The process is known to occur in a wide variety of organisms, including embryos of mammals and other vertebrates. The use of these dsRNAs (or recombinantly produced or chemically synthesized oligonucleotides of the same or similar nature) enables the targeting of the GlyRS mRNAs, for example, GARS in humans, for degradation in mammalian cells. Use of long dsRNAs in mammalian cells to elicit RNAi is not desired in some embodiments because of the deleterious effects of the interferon response. Specific targeting of a particular gene function, which is possible with short oligonucleotides (e.g., 19-23 nt RNA), is useful in functional genomic and therapeutic applications.

Some embodiments disclosed herein provide small interfering RNA (siRNA) sequences, RNA interfering vectors, and RNA interfering lentiviruses that are directed at a GlyRS gene, e.g., the human GARS gene. In some embodiments, provided are isolated double-stranded ribonucleic acid (dsRNA) molecules that inhibit expression of a GlyRS protein, wherein a first strand of the dsRNA is substantially identical to at least 19 consecutive nucleotides of the GlyRS gene, and a second strand of the dsRNA is substantially complementary to the first strand.

In some embodiments, the dsRNA molecules are small hairpin RNA (shRNA) molecules. The following is a list of exemplary shRNAs targeting human GARS gene:

shGARS2277
(SEQ ID NO: 3)
5′-GCATGGAGTATCTCACAAAGT
shGARS-696
(SEQ ID NO: 4)
5′ GCCCAAAGATGATATTGTAGA
shGARS-1324
(SEQ ID NO: 5)
5′ GCTGCCCAGATTGGAAATTCT

In some embodiments, the shRNA may comprise a sense fragment, which comprises a nucleotide sequence substantially identical to a target sequence in the GlyRS gene, and an antisense fragment, wherein the sense and antisense fragments are separated by a loop fragment, wherein the loop fragments may comprise a sequence selected from the group consisting of UUCAAGAGA, AUG, CCC, UUCG, CCACC, CTCGAG, AAGCUU and CCACACC.

In some embodiments, an siRNA target sequence may be designed on the basis of the human GARS gene, preferably 15 to 27, more preferably 19 to 23, and optimally 19, 20 or 21, consecutive bases.

The following is a list of non-limiting exemplary siRNAs targeting human GARS gene:

(SEQ ID NO: 6)
GGCGTTACAGCCCAAAGAT
(SEQ ID NO: 7)
GCCCAAAGATGATATTGTA
(SEQ ID NO: 8)
CCTGGAGGCAGCACTTTAT
(SEQ ID NO: 9)
GCTGCCCAGATTGGAAATT
(SEQ ID NO: 10)
GGAGCAATTGGTAAGGCAT
(SEQ ID NO: 11)
GCAATTGGTAAGGCATATA
(SEQ ID NO: 12)
CCGAATGTAATTGAACCTT
(SEQ ID NO: 13)
GCCTGGGTAGGATCATGTA
(SEQ ID NO: 14)
GGAGATGAACAGAGAACAT
(SEQ ID NO: 15)
GCATGGAGTATCTCACAAA
(SEQ ID NO: 16)
CCAGAATGTGGCAGACCTT
(SEQ ID NO: 17)
CCTGGGTAGGATCATGTAT
(SEQ ID NO: 18)
GGCCCAGCTTGATAACTAT
(SEQ ID NO: 19)
GGGTACTTGAGACCAGAAA
(SEQ ID NO: 20)
GGCAGAAATTGAGCACTTT
(SEQ ID NO: 21)
GGAAGAAGTTGTTCCGAAT
(SEQ ID NO: 22)
GGATCATGTATACGGTATT
(SEQ ID NO: 23)
GGCATGGAGTATCTCACAA
(SEQ ID NO: 24)
CCTATGCTTTGAAGGTTCT
(SEQ ID NO: 25)
GCTTTGAAGGTTCTCGTGT
(SEQ ID NO: 26)
TCAGAGCTGTGTCCCTGAA
(SEQ ID NO: 27)
GCAAATCTGTTCGCTCGCA
(SEQ ID NO: 28)
GCGGCGATTTCATCATGCT
(SEQ ID NO: 29)
GCGATTTCATCATGCTCCG
(SEQ ID NO: 30)
CCCAAAGATGATATTGTAG
(SEQ ID NO: 31)
GCTGTGCTTTGAAGAACAA
(SEQ ID NO: 32)
GCACTTTATCCAAGAGGAA
(SEQ ID NO: 33)
TCCCATTACTGGAAATGAT
(SEQ ID NO: 34)
GCTATTTCATTGGCCGCAT
(SEQ ID NO: 35)
GCATCTACCTCTACCTCAC

Some embodiments disclosed herein provide siRNA molecules that mediate RNAi. The siRNA molecules disclosed herein can also comprise a 3′ hydroxyl group. The siRNA molecules can be single-stranded or double stranded; such molecules can be blunt ended or comprise overhanging ends (e.g., 5′, 3′). In some embodiments, the siRNA molecule is double stranded and either blunt ended or comprises overhanging ends.

In some embodiments, at least one strand of the siRNA molecule has a 3′ overhang from about 1 to about 6 nucleotides (e.g., pyrimidine nucleotides, purine nucleotides) in length. In some embodiments, the 3′ overhang is from about 1 to about 5 nucleotides, from about 1 to about 3 nucleotides and from about 2 to about 4 nucleotides in length. In one embodiment, the siRNA molecule is double stranded, one strand has a 3′ overhang and the other strand can be blunt-ended or have an overhang. In the embodiment in which the siRNA molecule can be double stranded and both strands comprise an overhang, the length of the overhangs may be the same or different for each strand. In a particular embodiment, the siRNA of the present invention comprises 21 nucleotide strands which are paired and which have overhangs of from about 1 to about 3, particularly about 2, nucleotides on both 3′ ends of the siRNA. In order to further enhance the stability of the RNA of the present invention, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The siRNA molecules disclosed herein can be obtained using a number of techniques known to those of skill in the art. For example, the siRNA can be chemically synthesized or recombinantly produced using methods known in the art. The siRNA can also be obtained using an in vitro system. The in vitro system can also be used to obtain siRNA of about 19 to about 23 nucleotides in length which mediates RNA interference of the mRNA of the GlyRS gene.

The method of obtaining the siRNA sequence using the in vitro system can further comprise isolating the RNA sequence from the combination. The siRNA molecules can be isolated using a number of techniques known to those of skill in the art. For example, gel electrophoresis can be used to separate siRNA from the combination, gel slices comprising the RNA sequences removed and RNAs eluted from the gel slices. Alternatively, non-denaturing methods, such as non-denaturing column chromatography, can be used to isolate the RNA produced. In addition, chromatography (e.g., size exclusion chromatography), glycerol gradient centrifugation, affinity purification with antibody can be used to isolate siRNAs. The RNA-protein complex isolated from the in vitro system can also be used directly in the methods described herein (e.g., method of mediating RNAi of mRNA of the GlyRS gene).

The siRNAs described herein can be used in a variety of ways. For example, the siRNA molecules can be used to mediate RNA interference of mRNA of a gene in a cell or organism. In a specific embodiment, the siRNA is introduced into human cells or a human in order to mediate RNA interference in the cells or in cells in the individual, such as to prevent or treat a disease or undesirable condition. In this method, a gene (or genes) that cause or contribute to the disease or undesirable condition is targeted and the corresponding mRNA (the transcriptional product of the targeted gene) is degraded by RNAi. In this embodiment, an siRNA that targets the corresponding mRNA (the mRNA of the targeted gene) for degradation is introduced into the cell or organism. The cell or organism is maintained under conditions under which degradation of the corresponding mRNA occurs, thereby mediating RNA interference of the mRNA of the gene in the cell or organism. In the event that the siRNA is introduced into a cell in which RNAi does not normally occur, the factors needed to mediate RNAi are introduced into such a cell or the expression of the needed factors is induced in such a cell. Alternatively, siRNA produced by other methods (e.g., chemical synthesis, recombinant DNA production) to have a composition the same as or sufficiently similar to an siRNA known to mediate RNAi can be similarly used to mediate RNAi. Such siRNAs can be altered by addition, deletion, substitution or modification of one or more nucleotides and/or can comprise non-nucleotide materials.

At the same time, disclosed herein is a GlyRS RNAi lentivirus and the preparation and application thereof. A nucleic acid construct that expresses the above-described siRNA may be constructed by means of gene cloning and packaged with a lentivirus that expresses the above-described siRNA. Cell experiments prove that the above-described siRNA sequence can specifically silence the expression of endogenous GlyRS genes in cells.

In some embodiments, a DNA sequence encoding the above-described siRNA may be contained in a lentivirus vector. In some embodiments, the lentivirus vector may further comprise a promoter sequence. In some embodiments, the lentivirus vector may further comprise a nucleotide sequence encoding a detectable marker in the cell, wherein the detectable marker may be a green fluorescent protein (GFP). In some embodiments, the lentivirus vector may be selected from the group consisting of pLentiLox 3.7, pLKO.1-puro, pLKO.1-CMV-tGFP, pLKO.1-puro-CMV-tGFP, pLKO.1-CMV-Neo, pLKO.1-Neo, pLKO.1-Neo-CMV-tGFP, pLKO.1-puro-CMV-TagCFP, pLKO.1-puro-CMV-TagYFP, pLKO.1-puro-CMV-TagRFP, pLKO.1-puro-CMV-TagFP635, pLKO.1-puro-UbC-TurboGFP, pLKO.1-puro-UbC-TagFP635, pLKO-puro-IPTG-1×LacO, pLKO-puro-IPTG-3×LacO, pLP1, pLP2, pLP/VSV-G, pENTR/U6, pLenti6/BLOCK-iT-DEST, pLenti6-GW/U6-laminshrna, pcDNA1.2/V5-GW/lacZ, pLenti6.2/N-Lumio/V5-DEST, pGCSIL-GFP and Lenti6.2/N-Lumio/V5-GW/lacZ. In some embodiments, the siRNA lentiviruses designed for GlyRS stably and specifically lower GlyRS expression and effectively inhibit neddylation.

### Methods of Identifying Inhibitors of Neddylation

Disclosed herein are methods of identifying an inhibitor of neddylation. The method, in some embodiments, comprises: providing one or more compounds; testing the one or more compounds for their ability to reduce or inhibit neddylation. In some embodiments, the compounds can be tested for their ability to reduce the binding between a GlyRS protein and NEDD8-conjugated Ubc12. In some embodiments, the methods comprise identifying one or more compounds that have the ability to reduce or inhibit the binding between the GlyRS protein and NEDD8-conjugated Ubc12 as inhibitors of neddylation. The method can further comprise testing the one or more compounds for their ability to reduce or inhibit aminoacylation activity of the GlyRS protein.

The potential inhibitory or binding effect of a chemical compound on GlyRS may be analyzed prior to its actual synthesis and testing by the use of computer modelling techniques. If the theoretical structure of the given compound suggests insufficient interaction and association between it and GlyRS, synthesis and testing of the compound is obviated. However, if computer modelling indicates a strong interaction, the molecule may then be synthesized and tested for its ability to bind to GlyRS and inhibit using a suitable assay. In this manner, synthesis of inoperative compounds may be avoided. An inhibitory or other binding compound of GlyRS may be computationally evaluated and designed by means of a series of steps in which chemical entities or fragments are screened and selected for their ability to associate with the individual binding pockets or other areas of GlyRS.

One of skill in the art may use various methods to test chemical entities or fragments for their ability to associate with GlyRS and more particularly with the individual binding pockets of the GlyRS active site or accessory binding site. PCT Patent Publication No. WO 2000058345 A1 describes design of GlyRS binding compounds using computer modeling, the content of which is hereby incorporated by reference in its entirety. In some embodiments, a known GlyRS inhibitor, such as Gly-SA, may be used as a starting point for designing derivative compounds that inhibit GlyRS.

In some embodiments, small molecule inhibitors of neddylation may be identified using standard techniques. For example, candidate compounds may be used in binding assays using conventional formats to screen inhibitors of interaction between GlyRS and a component of the neddylation pathway. One particularly suitable assay format includes the enzyme-linked immunosorbent assay (ELISA). Other assay formats may be used; these assay formats are not a limitation on the present disclosure.

In another aspect, the structure of the GlyRS protein permits the design and identification of synthetic compounds and/or other molecules which are characterized by the conformation of the GlyRS protein. Using known computer systems, the coordinates of the GlyRS protein structure may be provided in machine readable form, the test compounds designed and/or screened and their conformations superimposed on the structure of the GlyRS protein. Subsequently, suitable candidates identified as above may be screened for the desired GlyRS protein inhibitory bioactivity, stability, and the like. Once identified and screened for biological activity, these inhibitors may be used therapeutically or prophylactically to block GlyRS protein activity, and thus, neddylation in a cell.

In some embodiments, the identified inhibitor of neddylation binds to the catalytic domain of the GlyRS protein. The GlyRS protein can be, for example, a mammalian GlyRS protein, particularly a human GlyRS protein. In some embodiments, the human GlyRS protein comprises an amino acid sequence having at least 90% sequence identity to the amino acid sequence set forth in SEQ ID NO: 2.

In some embodiments, the compound that inhibits binding between the GlyRS protein and NEDD8-conjugated Ubc12 binds to a sequence comprising amino acids 84-93 of SEQ ID NO: 2. In some embodiments, the compound that inhibits binding between the GlyRS protein and NEDD8-conjugated Ubc12 binds to one or more of amino acids 84-93 of SEQ ID NO: 2. In some embodiments, the compound that inhibits binding between the GlyRS protein and NEDD8-conjugated Ubc12 binds to a sequence comprising amino acids 232-238 of SEQ ID NO: 2. In some embodiments, the compound that inhibits binding between the GlyRS protein and NEDD8-conjugated Ubc12 binds to one or more of amino acids 232-238 of SEQ ID NO: 2.

### Pharmaceutical Compositions and Methods of Administration

Some embodiments disclosed herein provide pharmaceutical compositions comprising one or more GlyRS inhibitors and a pharmaceutically acceptable excipient for the treatment of cancers. The GlyRS inhibitors can be any of the GlyRS inhibitors disclosed herein. As disclosed herein, the GlyRS inhibitors may be small molecules, nucleic acids, antibodies, peptides, or any combination thereof. In some embodiments, the GlyRS inhibitors may be small molecule compounds that inhibit or reduce the interaction between a GlyRS protein and components of the neddylation pathway, or isolated double-stranded dsRNA molecules that inhibit or reduce expression of a GlyRS protein. In some embodiments, the GlyRS inhibitor reduces or inhibits GlyRS functions in aminoacylation and neddylation. In some embodiments, the GlyRS inhibitor does not significantly reduce or inhibit GlyRS function in aminoacylation. In some embodiments, the GlyRS inhibitor only reduces or inhibits GlyRS function in neddylation.

In addition to the one or more GlyRS inhibitors, the pharmaceutical compositions disclosed herein can comprise one or more therapeutic agents. Non-limiting examples of therapeutic agents include chemotherapeutic agents, cancer drugs, or prodrugs or pharmaceutically acceptable salts thereof. The chemotherapeutic agents can be, for example, AZ-23, BMS-754807, bosutinib, cabozantinib, ceritinib, crizotinib, entrectinib, foretinib, GNF 5837, GW441756, imatinib mesylate, K252a, LOXO-101, MGCD516, nilotinib hydrochloride monohydrate, NVP-TAE684, PF-06463922, rebastinib, staurosporine, sorafenib tosylate, sunitinib malate, and TSR-011.[0089] Also provided are pharmaceutically acceptable prodrugs of the pharmaceutical compositions, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the agent). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in Bundgaard, Design of Prodrugs (Elsevier Press, 1985).

Also provided are pharmaceutically active metabolites of the pharmaceutical compositions, and uses of such metabolites in the methods of the invention. A “pharmaceutically active metabolite” means a pharmacologically active product of metabolism in the body of a compound or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any suitable formulation of the compounds described herein can be prepared. See, generally, Remington's Pharmaceutical Sciences, (2000) Hoover, J. E. editor, 20th edition, Lippincott Williams and Wilkins Publishing Company, Easton, Pa., pages 780-857. A formulation is selected to be suitable for an appropriate route of administration. Some routes of administration are oral, parenteral, by inhalation, topical, rectal, nasal, buccal, vaginal, via an implanted reservoir, or other drug administration methods. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The pharmaceutical compositions as described herein are generally soluble in organic solvents such as chloroform, dichloromethane, ethyl acetate, ethanol, methanol, isopropanol, acetonitrile, glycerol, N,N-dimethylformamide, N,N-dimetheylaceatmide, dimethylsulfoxide, or any combination thereof. In one embodiment, the present invention provides formulations prepared by mixing an agent with a pharmaceutically acceptable carrier. In one aspect, the formulation may be prepared using a method comprising: a) dissolving a described agent in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

The pharmaceutical compositions comprising GlyRS inhibitors and disclosed herein can be used in combination with a pharmaceutical composition comprising one or more therapeutic agents. As used herein the terms “combination” and “in combination with” mean the administration of a therapeutic agent described herein together with at least one additional pharmaceutical or medicinal agent (e.g., an anti-cancer agent), either sequentially or simultaneously. For example, the term encompasses dosing simultaneously, or within minutes or hours of each other, or on the same day, or on alternating days, or dosing the therapeutic agent described herein on a daily basis, or multiple days per week, or weekly basis, for example, while administering another compound such as a chemotherapeutic agent on the same day or alternating days or weeks or on a periodic basis during a time simultaneous therewith or concurrent therewith, or at least a part of the time during which the therapeutic agent described herein is dosed.

Pharmaceutical compositions for use in accordance with the present disclosure can be manufactured and/or formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Suitable routes of administration of the pharmaceutical compositions disclosed herein include, for example, oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The compound or combination of compounds disclosed herein can also be administered in sustained or controlled release dosage forms, including depot injections, osmotic pumps, pills, transdermal (including electrotransport) patches, and the like, for prolonged and/or timed, pulsed administration at a predetermined rate.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized. For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compound(s) or combination of compounds disclosed herein can be formulated readily by combining the active compound with pharmaceutically acceptable carriers well known in the art. Such carriers enable the compound or combination of compounds disclosed herein to be formulated as tablets, film coated tablets, pills, dragees, capsules, liquids, gels, get caps, pellets, beads, syrups, slurries, suspensions and the like, for oral ingestion by a patient to be treated. Pharmaceutical preparations for oral use can be obtained by combining the active compound with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. In addition, stabilizers can be added. All formulations for oral administration should be in dosages suitable for such administration. In some embodiments, formulations of the compound(s) or combination of compounds disclosed herein with an acceptable immediate release dissolution profile and a robust, scalable method of manufacture are disclosed.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner. For administration by inhalation, the compound or combination of compounds disclosed herein is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the active compounds in water-soluble form, such as eyedrops, or in gellan gum or hydrogels; ophthalmic ointments; ophthalmic suspensions, such as microparticulates, drug-containing small polymeric particles that are suspended in a liquid carrier medium, lipid-soluble formulations, microspheres, and ocular inserts. Suitable pharmaceutical formulations are most often and preferably formulated to be sterile, isotonic and buffered for stability and comfort. Pharmaceutical compositions for intranasal delviery may also include drops and sprays often prepared to simulate in many respects nasal secretions to ensure maintenance of normal ciliary action. As disclosed in Remington's Pharmaceutical Sciences, 18th Ed., Mack Publishing Co., Easton, Pa. (1990), which is incorporated herein by reference in its entirety, and well-known to those skilled in the art, suitable formulations are often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The compound(s) or combination of compounds disclosed herein may also be formulated in rectal compositions such as suppositories or retention enemas, e.g., containing conventional suppository bases such as cocoa butter or other glycerides.

In addition to the formulations described previously, the compound or combination of compounds disclosed herein may also be formulated as a depot preparation. Such long acting formulations may be administered by implantation (for example subcutaneously or intramuscularly) or by intramuscular injection. Thus, for example, the compound or combination of compounds disclosed herein may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. In some embdoimetns, small hydrophobic organic molecules may be directly administered intracellularly. Additional therapeutic or diagnostic agents may be incorporated into the pharmaceutical compositions. Alternatively or additionally, pharmaceutical compositions may be combined with other compositions that contain other therapeutic or diagnostic agents.

### Methods of Reducing Neddylation

Some embodiments disclosed herein provide methods of reducing neddylation in a cell, in a cell population, and/or in a subject. In some embodiments, the methods comprise contacting a cell, a cell population, and/or a subject with a composition comprising a GlyRS inhibitor, wherein the level of neddylation is decreased in the cell, the cell population, and/or the subject. In some embodiments, the methods comprise acquiring knowledge of the level of neddylation in the cell, the cell population, and/or the subject before and/or after contacting the cell, the cell population, and/or the subject with the composition. In some embodiments, the methods comprise identifying a cell, a cell population, and/or a subject having undesired level of neddylation before and/or after contacting the cell, the cell population, and/or the subject with the composition.

The composition can be, for example, a pharmaceutical composition. The GlyRS inhibitor can be any of the GlyRS inhibitor disclosed herein. The GlyRS inhibitor may inhibit GlyRS functions in aminoacylation and neddylation. In some embodiments, the GlyRS inhibitor does not significantly inhibit GlyRS function in aminoacylation. In some embodiments, the GlyRS inhibitor is an inhibitor for a mammalian GlyRS protein (e.g., a human GlyRS protein). In some embodiments, the GlyRS inhibitor is GlySA or a derivative thereof.

The cell can be, for example a mammalian cell (e.g., a human cell). The cell population can comprise, or consist of, mammalian cells (e.g., human cells). The cell or the cell population can be present in cell culture, a tissue, in an organ, or in a body of a subject. Contacting the cell or the cell population with the composition can be performed in vitro, ex vivo, in vivo, or a combination thereof.

### Methods of Inhibiting Cell Proliferation

Some embodiments disclosed herein provide methods of inhibiting or reducing cell proliferation. The methods, in some embodiments, comprise: contacting a cell, a cell population, or a subject with a pharmaceutical composition comprising a GlyRS inhibitor, wherein the proliferation of the cell, one or more cells present in the cell population or the subject is inhibited. As described herein, the inhibition of cell proliferation can be complete or partial. In some embodiments, a method of reducing cell proliferation is provided.

In some embodiments, the methods comprise acquiring knowledge of the level of cell proliferation in the cell, the cell population, and/or the subject before and/or after contacting the cell, the cell population, and/or the subject with the composition. In some embodiments, the methods comprise identifying a cell, a cell population, and/or a subject having undesired level of cell proliferation before and/or after contacting the cell, the cell population, and/or the subject with the composition.

The NEDD8 pathway has been shown to be essential for cellular function, through its critical role in mediating the ubiquitination by CRLs of numerous proteins involved in cell cycle progression and cell growth and survival. The relevance of the NEDD8 conjugation pathway in various cancer therapies has been discussed in Soucy et al., The NEDD8 Conjugation Pathway and Its Relevance in Cancer Biology and Therapy. Genes Cancer 1, 708-16 (2010), the content of which is hereby expressly incorporated by reference in its entirety. Table 1 of Soucy et al. summarizes various substrate proteins of the cullin-RING ligases and their associations with cancer, and is reproduced below. Additional substrates that are involved in tumorigenesis include: tumor suppressor NF2, which encodes Merlin, p53, Mdm2, epidermal growth factor receptor, VHL tumor suppressor protein, L11, or any combination thereof.

TABLE 1
Substrate Proteins of the Cullin-RING Ligases (CRLs) and Their Associations with Cancer

| Substrate | Role | CRL | Association with Cancer |
|---|---|---|---|
| Cdt-1 | DNA replication licensing factor | CRL1^{Skp2}/ | Dysregulated expression in human |
| | | CRL4^{cdt2} | tumors: overexpression linked to |
| | | | genomic instability |
| p27 | Cyclin-dependent kinase inhibitor | CRL1^{Skp2}/ | Reduced levels due to increased |
| | | CRL4 | degradation: seen in multiple cancers, |
| | | | associated with poor prognosis |
| plκBα | Inhibitor of NF-κB | CRL1^{βTrCP1} | Constitutive IKK activity results in |
| | | | plκBα degradation and constitutive NF- |
| | | | KB activity, demonstrated in human |
| | | | cancers including ABC-DLBCLNF-κB |
| | | | signaling associated with |
| | | | chemoresistance in various tumor types |
| NRF2 | Stress-response transcription factor | CRL3^{Keap1} | Overexpressed in multiple human |
| | | | cancers; may also be associated with |
| | | | acquired chemoresistance |
| HIF-1α | Stress (hypoxia)-response | CRL2^{VHL} | Aberrant expression of VHL tumor |
| | transcription factor | | suppressor protein results in |
| | | | development of a variety of tumors; |
| | | | HIF-1α important for tumor survival |
| Cyclin E | Cell cycle regulator | CRL1^{Fbw7} | Aberrant cyclin E expression associated |
| | | | with tumor development and |
| | | | progression in breast cancer and with |
| | | | various other cancers |
| c-Jun | AP1 transcription factor | CRL1^{Fbw7} | c-Jun identified as an oncoprotein |
| β-catenin | Transcription factor | CRL1^{βTrCP} | Upregulated in various cancers |
| | | | including colon and prostate cancer and |
| | | | melanoma |
| Cdc25A | Activator of cyclin-dependent | CRL1^{βTrCP} | Upregulated in various cancers |
| | kinase complexes CDK2/cyclin A | | |
| | and CDK2/cyclin E | | |
| Emi1 | APC^{Cdc20} inhibitor | CRL1^{βTrCP/Slimb} | Possible involvement in development |
| | | | of ovarian clear cell carcinoma |
| c-Myc | Cell proliferation regulator | CRL1^{Fbw7} | c-Myc identified as an oncoprotein |
| mTOR | Cell growth and proliferation | CRL1^{Fbw7} | Dysregulated mTOR signaling in |
| | signaling | | various human tumors |
| BimEL | Tumor suppressor; proapoptotic | CRL1^{βTrcP} | Levels decreased in transformed cells |
| | BH3-only protein | | through enhanced degradation |

Neddylation of the cullin proteins activates the E3 ligases for ubiquitination and promotes the degradation of their downstream targets, including key regulators of cell cycle. For example, c-Myc, c-Jun, cyclin E, Emil, Cdt-1, pIκBα, NRF2, HIF-1α, β-catenin, Cdc25A, mTOR, BimEL and p27.

Cell proliferation can be inhibited in various types of cells, including animal cells and plant cells. In some embodiments, the cell is a mammalian cell. The extent by which cell proliferation is reduced can vary. In some embodiments, the proliferation of the cell is reduced by at least 20%, 30%, 40%, 50%, 60%, 70%, 80%, 90%, 95%, or 99%.

The composition can be, for example, a pharmaceutical composition. The GlyRS inhibitor can be any of the GlyRS inhibitor disclosed herein. The GlyRS inhibitor may inhibit GlyRS functions in aminoacylation and neddylation. In some embodiments, the GlyRS inhibitor does not significantly inhibit GlyRS function in aminoacylation. In some embodiments, the GlyRS inhibitor is an inhibitor for a mammalian GlyRS protein (e.g., a human GlyRS protein). In some embodiments, the GlyRS inhibitor is GlySA or a derivative thereof.

The cell can be, for example a mammalian cell (e.g., a human cell). The cell population can comprise, or consist of, mammalian cells (e.g., human cells). The cell or the cell population can be present in cell culture, a tissue, in an organ, or in a body of a subject. Contacting the cell or the cell population with the composition can be performed in vitro, ex vivo, in vivo, or a combination thereof.

### Methods of Treating Cancer

Also disclosed herein are methods of treating or ameliorating cancer in a subject by administering a therapeutically effective amount of a pharmaceutical composition comprising a GlyRS inhibitor to the subject. As disclosed herein, the GlyRS inhibitor may be capable of inhibiting or reducing interaction between the GlyRS protein and the components of the neddylation pathway, thereby reduce proliferation of the cancer cells. For example, the methods may be used for treating or ameliorating a solid tumor or a hematological malignancy, for example, a cancer that is selected from the group consisting of breast cancer, cervical cancer, colon cancer, liver cancer, prostate cancer, melanoma, ovarian cancer, lung cancer, renal cell carcinoma, Schwannoma, mesothelioma, acute myeloid leukemia, multiple myeloma, non-Hodgkin lymphoma, and a combination thereof.

The GlyRS inhibitor can be any of the GlyRS inhibitor disclosed herein. The GlyRS inhibitor may inhibit GlyRS functions in aminoacylation and neddylation. In some embodiments, the GlyRS inhibitor does not significantly inhibit GlyRS function in aminoacylation. In some embodiments, the GlyRS inhibitor is an inhibitor for a mammalian GlyRS protein (e.g., a human GlyRS protein). In some embodiments, the GlyRS inhibitor is GlySA or a derivative thereof.

To practice the methods disclosed herein, a GlyRS inhibitor, and/or an agent that prevents or reduces interaction between a GlyRS protein and a component of the neddylation pathway, and pharmaceutical compositions thereof may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

### EXAMPLES

Embodiments of the present application are disclosed in further detail in the following examples, which are not in any way intended to limit the scope of the present disclosure.

### Example 1

### Glycyl-tRNA Synthetase is Critical for Neddylation by Protecting Activated E2

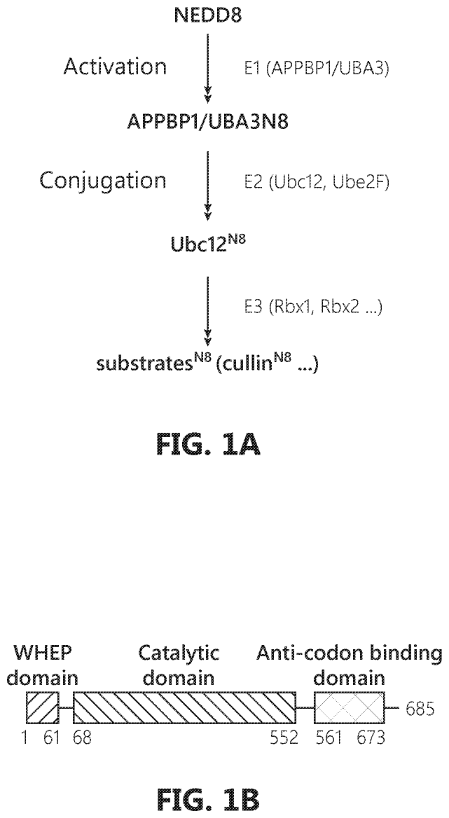

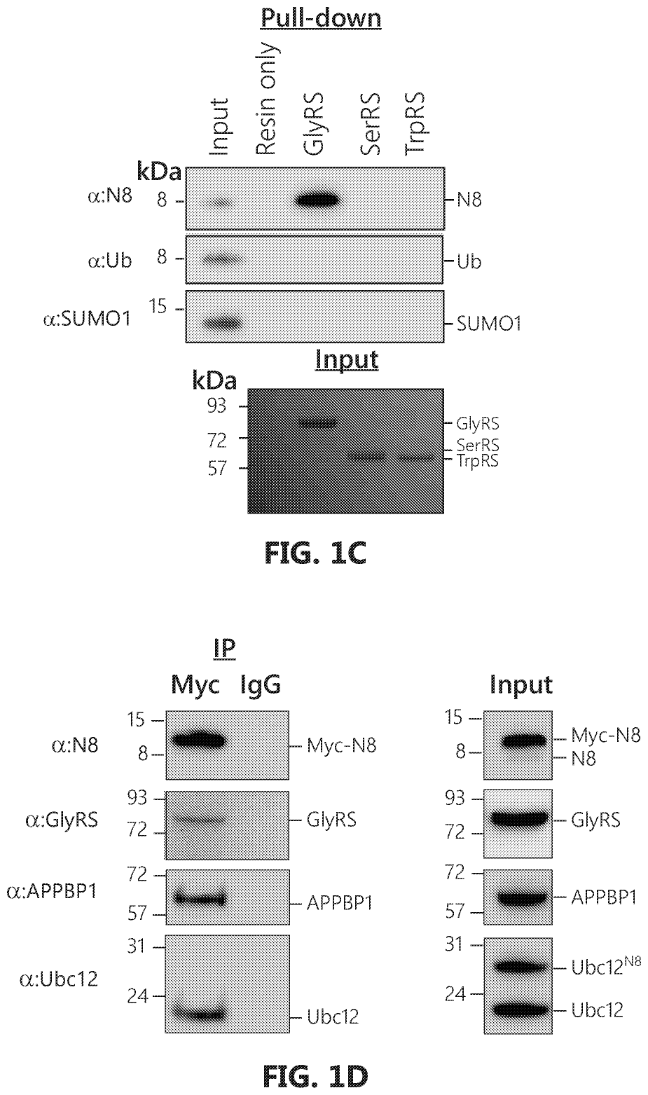

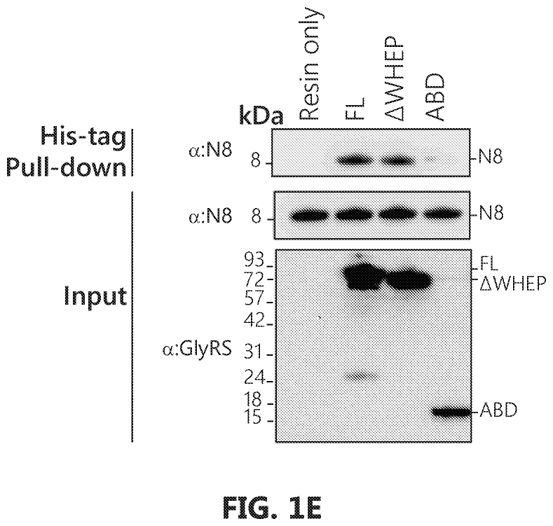

Ubiquitination and ubiquitin-like modifications are post-translational modifications in eukaryotes playing key roles in protein homeostasis and functions¹. Among them, neddylation-conjugating the ubiquitin-like protein NEDD8 to its target proteins—is an essential biological process in organisms from yeast to mammals to critically regulate cell cycle progression. Like ubiquitination, the modification is achieved through a sequential enzymatic cascade involving an activating enzyme (E1), a conjugating enzyme (E2), and a ligase (E3) (FIG. 1a). To date, one E1 (APPBP1/UBA3), two E2 (Ubc12 and Ubc2F), and several E3 ligases have been found for neddylation (FIG. 1a). Although many NEDD8 targets were reported⁸, the biological functions of neddylation so far have been primarily characterized in the context of its main target—the cullin family, a critical component of the ubiquitin E3 ligase family of cullin-RING ligases. Neddylation of the cullin proteins activates the E3 ligases for ubiquitination and promotes the degradation of their downstream targets, including key regulators of cell cycle.

Ubiquitin and most ubiquitin-like modifier proteins, including NEDD8, have a conserved C-terminal glycine that is used to activate, conjugate, and finally attach the modifiers to their targets. Structurally, the glycine residue is located at the tip of a flexible ‘tail’ protruding out from the central ubiquitin fold to provide with the accessibility. On the other hand, GlyRS is a member of the evolutionarily ancient aminoacyl-tRNA synthetase family essential for all living organisms. GlyRS catalyzes the aminoacylation reaction to attach glycine to the 3′ end of the cognate tRNAs to provide substrates for protein synthesis in the ribosome. Aminoacylation of tRNA is a two-step reaction: first, GlyRS activates glycine with ATP to generate Gly-AMP; then the glycyl moiety is transferred from Gly-AMP to the tRNA to make the ‘charged’ tRNA. Interestingly, the first step of this aminoacylation reaction is chemically equivalent to the first step activation reaction catalyzed by an E1 enzyme. Moreover, the specific amino acid binding pocket of a tRNA synthetase can be exploited for binding to a cognate amino acid residue on a protein to develop new functions.

By using biochemical and cell-based functional assays, a function connection between GlyRS and neddylation in human cells have been revealed. It turns out that GlyRS plays an important role in neddylation through direct interactions with multiple components of the neddylation pathway. Although it appears that GlyRS cannot function as an E1, it can bind to the APPBP1 subunit of E1 to capture and protect the activated E2 (NEDD8-conjugated Ubc12) to critically enhance neddylation. Knockdown of GlyRS, but not a different tRNA synthetase, decreases the global level of neddylation and delays cell cycle progression. This provides the first example of a translation factor directly functioning in posttranslational modification.

### Methods and Materials

### Plasmid Constructs and Protein Purification

GST-tagged or untagged APPBP1-UBA3, Ubc12, and NEDD8 proteins were purified as previously reported. GST-APPBP1 was obtained by injecting the purified GST-APPBP1/UBA3 into the Superdex 200 column and collected the fractions that contained only GST-APPBP1. N-terminal His-SUMO-tagged NEDD8 construct was generated by subcloning the pGEX2TK-NEDD8 into a modified pET28a vector and the protein was purified using a Nickel-NTA column followed by MonoQ ion exchange column. Ubiquitin and SUMO1 proteins (R&D) were purchased. His-tagged full-length and AWHEP GlyRS, SerRS, and TrpRS were purified as reported earlier. The ABD (V541-E685) of GlyRS was cloned, expressed, and purified in a similar way as for the full-length GlyRS. (The yield for ABD alone is higher than that for the full-length GlyRS). Tag-free full-length GlyRS, ΔF84-L93 (F84-L93 replaced by GGG), ΔI232-M238 (I232-M238 replaced by GG), AInsertionl (F147-F224 replaced by GSGSGG) and G526R GlyRS proteins were similarly prepared and had the N-terminal His-SUMO-tag removed by Ulp1 protease. Inserting the GlyRS sequence into a modified pET28a vector generated N-terminal GST-tagged GlyRS and the protein was purified by glutathione sepharose chromatography. GST-Cullin1_{cfd}/Rbx1 was purified by glutathione sepharose chromatography as well. Ube2F protein was purified by a Nickel-NTA column. The purity of proteins was examined by SDS-PAGE to be above 95%.

### Ubc12ⁿ⁸ Preparation and Stability Assay

The conjugated Ubc12 was prepared by mixing APPBP1/UBA3 (2 μM), Ubc12 (C111S; the mutant would form a stable ester linkage to NEDD8) (70 μM), and His-SUMO-tagged NEDD8 (100 μM). The mixture was incubated at 25° C. for 16 h in 50 mM NaOAc pH 5.5, 50 mM NaCl, 1 mM MgCl2, and 2 mM ATP. Ubc12^{N8} was then purified by gel filtration chromatography using a Superdex 200 column. The fractions containing only Ubc12^{N8} were concentrated and used for the study. The purity of proteins was examined by SDS-PAGE to be above 95%.

The wild type Ubc12 conjugation was performed in a similar manner except using wild type Ubc12 instead of Ubc12 c 111s at 25° C. for 16 h in 50 mM Tris pH 7.4, 50 mM NaCl, 1 mM MgCl2, and 2 mM ATP. Ubc12^{N8} was then purified by gel filtration chromatography using a Superdex 200 column. The fractions containing only Ubc12^{N8} were concentrated and used for the study. The purity of proteins was examined by SDS-PAGE to be above 95%. The stability assay was carried out by incubating Ubc12^{N8} (504) with GlyRS, BSA or same volume of PBS buffer at 37° for indicated time in the PBS buffer (PH7.4 supplemented with 5 mM DTT). Samples were then subjected to SDS-PAGE and stained with commassie blue.

Cell Culture and shRNA Knockdown

HEK293 and HeLa cells were cultured in DMEM media supplemented with 10% FBS. Short-hairpin RNA (shRNA) sequences targeting the human GlyRS (5′-GCATGGAGTATCTCACAAAGT-3′, SEQ ID NO: 3) or human SerRS (5′-GGCATAGGGACCCATCATTGA-3′, SEQ ID NO: 36) were inserted into the pLentiLox-hH1 plasmid, modified from the pLentiLox 3.7 plasmid to contain a H1 promoter (between Xba I and Xho I sites) to drive shRNA expression. All transfections were done with Lipofectamine 2000 (Invitrogen) and cells were harvested 48 hours after transfection.

### Cycloheximide Chasing Assay

HeLa cells at 80% confluence were transfected with pLentiLox-hH1 vectors containing either a scramble sequence or GlyRS specific sequence using Lipofectamine 2000. 48 hours after transfection, medium were replaced with that containing 30 μg/mL cycloheximide (Cellsignalling) or 20 uM MG-132 (#508338, Fisher) as indicated. MLN4924 (1-502, Bostonbiochem) samples are prepared by treating the cells with 0.2 uM MLN4924 for 24 hours and then followed by cycloheximide or MG132. Samples are collected and lysed with acid lysis buffer and later subjected to SDS-PAGE.

### Immunobloting and Antibodies

Cells were washed with PBS and then lysed with either the lysis buffer (#4930; Cell Signaling Technology) or the acid lysis buffer (50 mM HEPES, 150 mM NaCl, 0.1% (w/v) SDS, pH 6.0) supplemented with protease cocktail (Roche). The supernatant of the lysates was used for Western blotting. The antibodies used in this studies include anti-cullin1 (H213; Santa cruz) anti-UBA3 (F-10; Santa cruz), anti-GlyRS (B01P; Abnova), anti-SerRS (homemade), anti-V5 (R96-CUS; Invitrogen). Anti-NEDD8 (#2745), anti-Ubiquitin (#3936), anti-SUMO1 (#4930), anti-Ubc12 (#5641), anti-Ubc9 (#4918), anti-Flag (#2908), anti-UBA1(#4891), anti-UbcH7 (#3848), anti-UBA2 (#8688), anti-p27kip (#3698) and anti-α-Tubulin (#3873) are all from Cell Signaling. Immunoblots are quantified and analyzed using ImageJ. The integrated areas of the bands were normalized to that of the corresponding a-tubulin level. Error bars indicate standard deviation with n>3. P values are calculated by one-tailed Student's t test.

### Immunoprecipitation

2 μg of anti-V5 (R96-CUS; Invitrogen), anti-GlyRS (D-10; Santa Cruz), anti-myc (9E10; Santa Cruz) antibodies or mouse IgG (#5415, Cell Signaling) were coupled to 30 μl of protein G-sepharose (Amersham Biosciences) beads and used for immunoprecipitations. Supernatant of HEK293 cells lysates were then added and incubated with the antibodies for 3 h or overnight at 4° C. The G-sepharose beads were then washed four times with 1 mL of cold PBS buffer (pH 7.4). The beads-bound proteins were eluted and denatured with SDS-loading buffer and subjected to SDS-PAGE and Western blotting.

### FACS Analysis

HeLa cells were transfected with either pCDNA6-V5c vectors containing either GARS or G526R mutant using Lipofectaming 2000. 48 hours after transefection, cells were treated with medium containing 0.5 uM MLN4924 or same amount of DMSO. 24 hours after treatment, cells were washed once with sorting buffer (PBS supplemented with 1% FBS and 5 mM EDTA) and collected using 0.05% Trypsin. The mixture was then spinned at 500 g for 5 min to spin cells down to the pellets. Cells were then washed twice with sorting buffer and then suspended and fixed with 70% EtOH at 4 for 2 hours. After fixation, cells were washed twice with sorting buffer and suspended with the PI staining solution. Samples were later analyzed by flow cytometry (BD FACS Canto).

### Affinity Pull-Down Assay

Glutathione sepharose beads (GE Healthcare) were equilibrated with TEE buffer (50 mM Tris pH 7.9), 1 mM EDTA, and 1 mM EGTA). GST-fusion proteins were mixed with 50 μl of glutathione sepharose beads and incubated for 2 h at 4° C. and then washed with TEE buffer twice. Aliquots of the protein-bound beads were then incubated together with different forms of GlyRS for 2 h at 4° C. Finally, the beads were washed 5 times with washing buffer (20 mM Hepes pH 7.9, 150 mM NaCl, 0.5 mM EDTA, 10% Glycerol, 0.1% Triton X-100, and 1 mM DTT) and proteins were eluted with SDS sample buffer and analyzed by immunoblotting. Control experiments were performed with GST-coated beads. His-tag pull-down assay were carried out in a similar manner using purified his-tagged proteins and incubated with nickel-NTA beads (Qiagen).

### Hydrogen-Deuterium Exchange Mass Spectrometry

Solution-phase amide HDX was carried out with a fully automated system as described previously. Briefly, 4 μL of protein was diluted to 20 μL with D2O-comtaining buffer and incubated at 4° C. for 10, 30, 60, 900, or 3600 s. Samples were diluted to 50 μL with 3M urea, 1% TFA at 1° C. to denature the proteins and minimize back-exchange. Samples were then passed across an immobilized pepsin column at 50 μL/min in 0.1% TFA at 15° C. Resulting peptides were trapped on a C8 cartridge (Hypersil Gold, Thermo Fisher). Peptides were then gradient eluted (4% CH3CN to 40% CH3CN, 0.3% formic acid) at 1° C. across a 1 mm×50 mm C18 reversed phase HPLC column (Hypersil Gold, Thermo Fisher) and electrosprayed directly into an orbitrap mass spectrometer (either LTQ Orbitrap or Q-Exactive, Thermo Fisher). Data were processed with in-house software and visualized with PyMol (Schrödinger, LLC). To measure the difference in exchange rates, the average percent deuterium uptake for unbound GlyRS protein was calculated at all time points. From this value, the average percent deuterium uptake for GlyRS protein bound to NEDD8 was subtracted. Negative perturbation values indicate exchange rates are slower for GlyRS bound to NEDD8, which suggests the region is less accessible to amide exchange due to structural alteration or direct contact between GlyRS and NEDD8. GlyRS-Ubc12 interaction was analyzed in a similar way.

### Biolayer Interferometry

The dissociation constants (K_{d}) were obtained with biolayer interferometry by using an Octet QK system (ForteBio, Menlo Park, Calif., USA). Samples or buffer were dispensed into 96-well plates (Millipore, Billerica, Mass.) at 200 μL per well. Operating temperature was maintained at 30° C. Proteins were diluted into kinetic buffer (PBS with 0.1% BSA and 0.002% Tween-20) and immobilized on either anti-GST or Ni-NTA sensor tips. The other proteins were diluted using the same buffer into a range of different concentrations. Assays with Ubc12^{N8} were carried out using the acid kinetic buffer (50 mM NaOAc pH 5.5, 50 mM NaCl, 0.1% BSA, and 0.002% Tween-20). The raw data were processed by subtraction to reference cells and then aligned with baselines. The dissociation constants K_{d} were obtained by fitting the processed data using the 1:1 model in the Octet analysis software with R²>0.99.

### Molecular Modeling

The Ubc12^{N8}-GlyRS interaction is obtained by using the Patchdock server. GlyRS (PDB: 2PME) is assigned as the receptor and Ubc12^{N8} (PDB: 4P50 chain G&H) is assigned as the ligand. Clustering RMSD is set at 4.0. The GlyRS-APPBP1 interaction is modeled using similar settings with APPBP1/UBA3 (PDB: 2NVU chain A&B) assigned as the receptor and GlyRS assigned as the ligand. Molecular visualization and analysis were performed using PyMOL (The PyMOL Molecular Graphics System, Version 1.2r1, Schrödinger, LLC.).

### Results

### GlyRS Specifically Binds to NEDD8 Through the Catalytic Domain

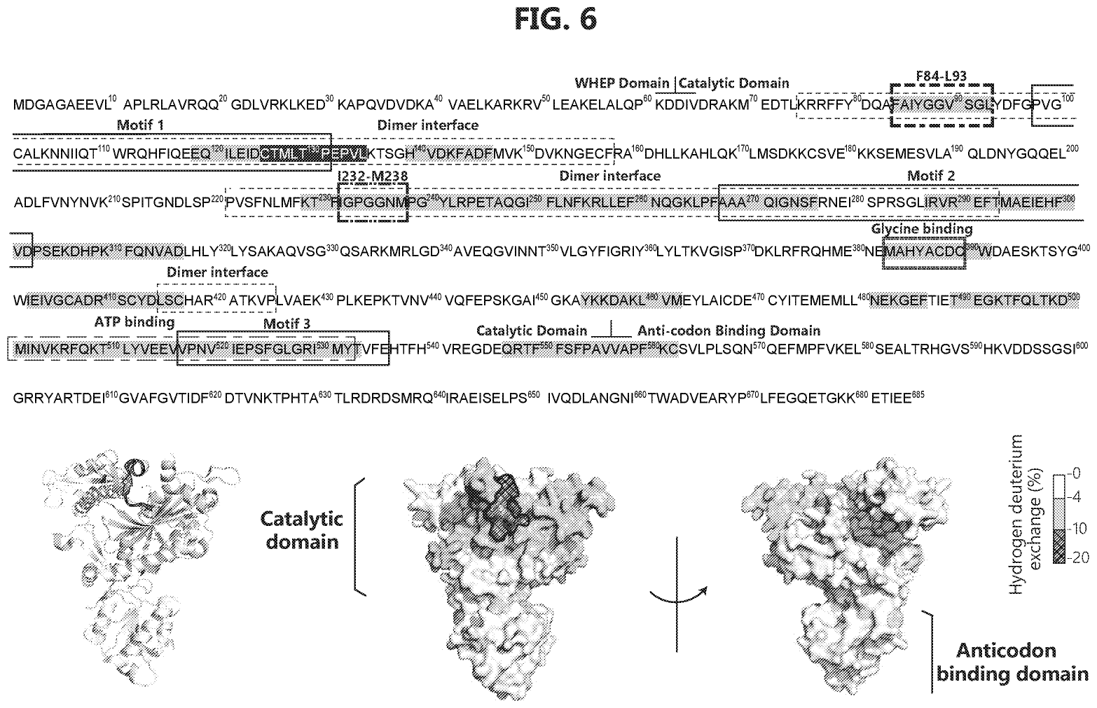

Human GlyRS is composed of three distinct domains: the N-terminal metazoan-specific WHEP domain, catalytic domain, and the C-terminal anticodon-binding domain (ABD) (FIG. 1b). GlyRS interaction with ubiquitin or ubiquitin-like proteins such as NEDD8 and SUMO1 were investigated. As shown in FIG. 1c, purified human GlyRS protein can specifically bind to NEDD8, but not ubiquitin or SUMO1. Two other human tRNA synthetases were tested side-by-side (SerRS and TrpRS) and showed no interaction with NEDD8, ubiquitin or SUMO1 (FIG. 1c). The GlyRS-NEDD8 interaction was verified in HEK293 cells by co-immunoprecipitation (FIG. 1d). Furthermore, by using truncated recombinant proteins, the interaction was mapped to the catalytic domain of GlyRS (FIG. 1e). Hydrogen-deuterium exchange (HDX) analysis (monitored by mass spectrometry) further confirmed that the catalytic domain is the site for interaction with NEDD8 (FIG. 6).

### GlyRS Plays a Critical Role in Neddylation

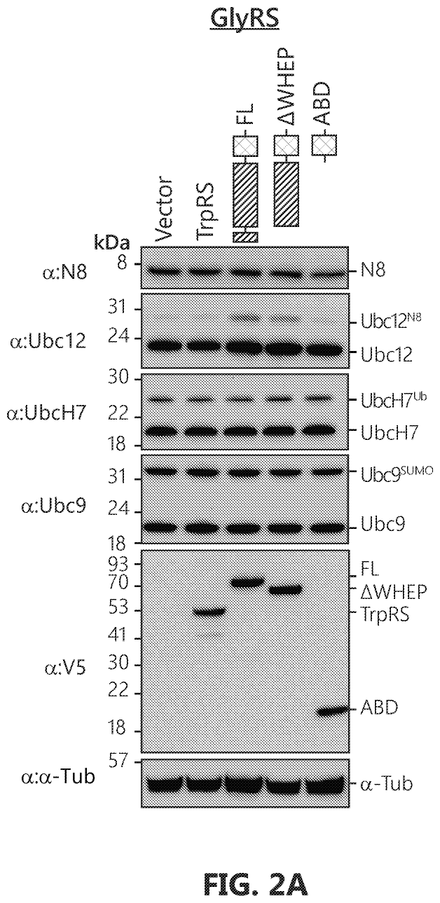

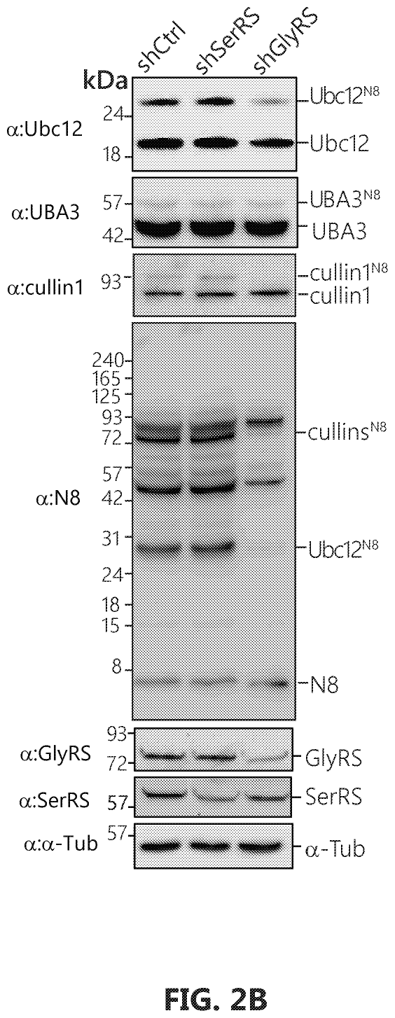

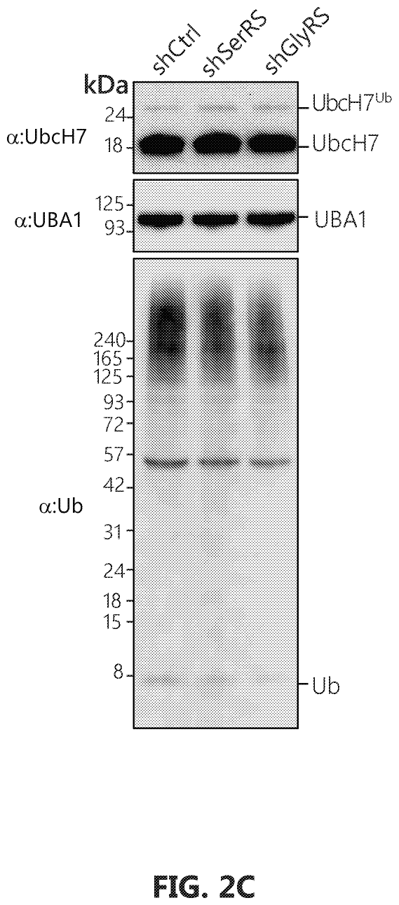

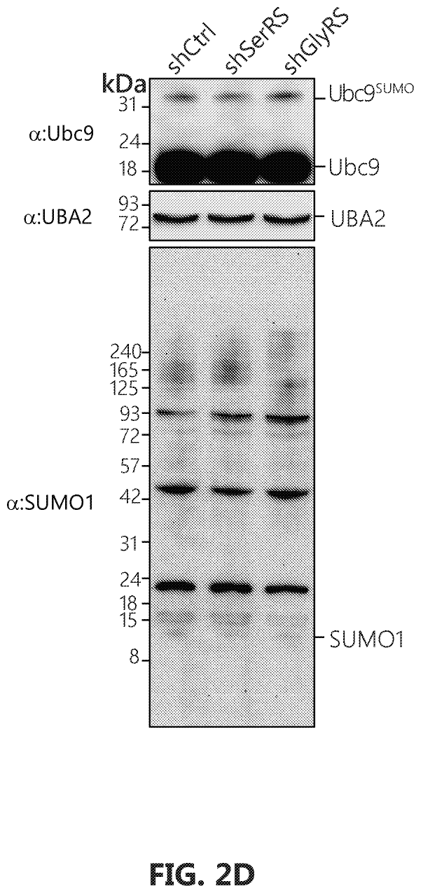

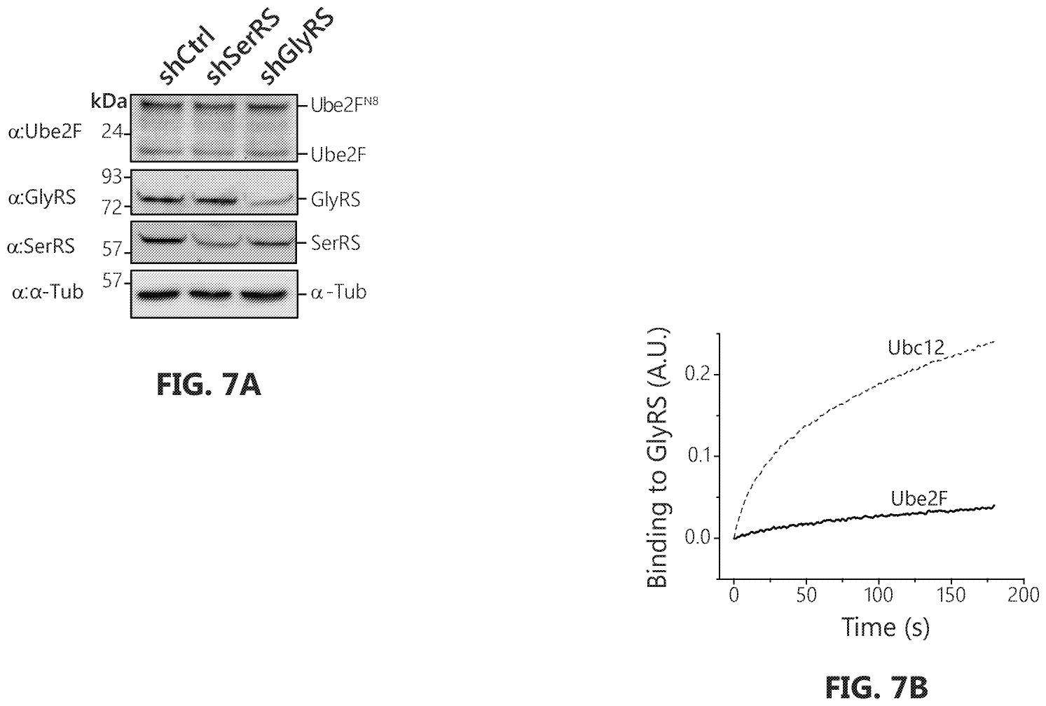

To test the effect of GlyRS in neddylation, GlyRS was ectopically expressed in HEK293 cells. The overexpression of GlyRS (but not TrpRS) increased the amount of NEDD8-conjugated Ubc12 (an E2 for neddylation), but not the ubiquitin-conjugated UbcH7 (an E2 for ubiquitination) and SUMO-conjugated Ubc9 (an E2 for sumoylation) (FIG. 2a). The core synthetase (AWHEP) was still active, while ABD domain alone did not have the effect (FIG. 2a), further highlighting the importance of the catalytic domain. Consistently, knockdown of GlyRS in HeLa cells led to significant decrease of NEDD8-conjugated Ubc12 (Ubc12^{N8}) (FIG. 2b), but not of the conjugated UbcH7 and Ubc9 (UbcH7^{ub} and Ubc9^{Sumo} (FIG. 2c,d). It was moted that the GlyRS knockdown had no effect on E1 neddylation (UBA3^{N8}) or the level of free NEDD8 (FIG. 2b). Also, GlyRS knockdown did not affect Ube2F (FIG. 7a), the other less frequently used E2 for neddylation⁸. Knockdown of a different tRNA synthetase (SerRS) had no effect on all E2s was tested (FIG. 2b-d and FIG. 7a). Importantly, knockdown of GlyRS, but not SerRS, also decreased the global level of neddylation, including neddylation of cullin proteins (FIG. 2b). These observations indicate that GlyRS plays an important role in the neddylation process, most likely through mechanism that enhances Ubc12 activity and/or protects the NEDD8-conjugated Ubc12.

GlyRS Strongly Binds to NEDD8-Conjugation Ubc12 and Protects it from Degradation

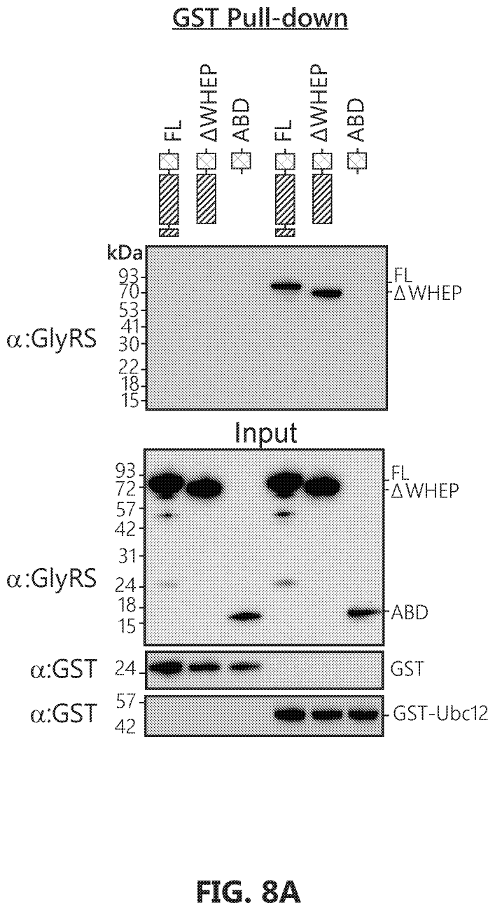

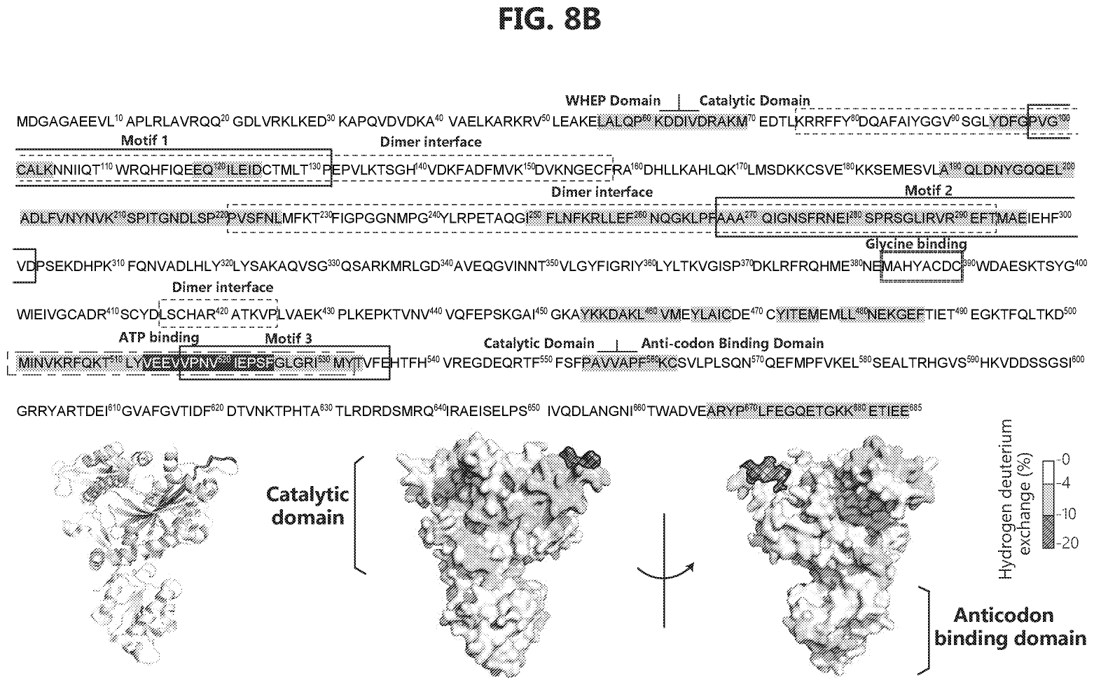

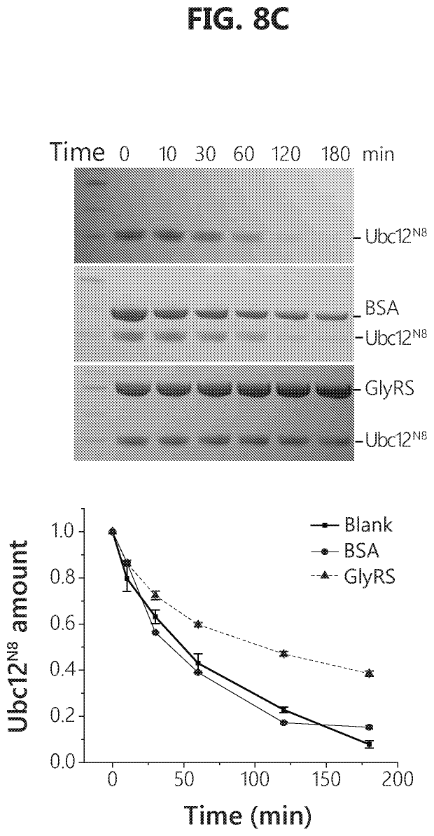

To understand how GlyRS promotes the cellular level of Ubc12^{N8}, whether GlyRS could interact with Ubc12 was tested. GST pull-down assay together with HDX analysis showed that GlyRS could bind to Ubc12 and again the catalytic domain is primarily responsible for this interaction (FIG. 8a & b). Consistent with the lack of effect on Ube2F conjugation when GlyRS was knocked down (FIG. 7a), there was very weak binding between Ube2F and GlyRS, if any (FIG. 7b).

As a conjugating enzyme, Ubc12 exists in two forms in the cell, the apo form and the conjugated form Ubc12^{N8}. The latter form is achieved by linking a NEDD8 molecule, transferred from the activating enzyme APPBP1/UBA3, to the catalytic cysteine residue (Cys111) of Ubc12 via a thioester bond. Because GlyRS can interact with both Ubc12 and NEDD8, it was tempting to test if GlyRS could bind to Ubc12^{N8} and protect it from degradation. Thioester bonds, like the one that links NEDD8 to Ubc12, are common intermediates in biological reactions and are highly labile.

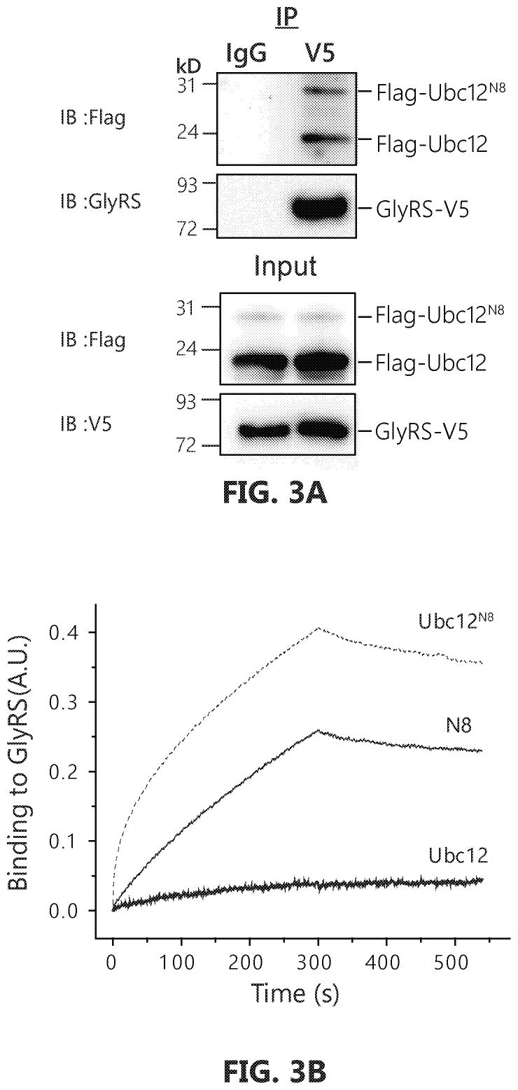

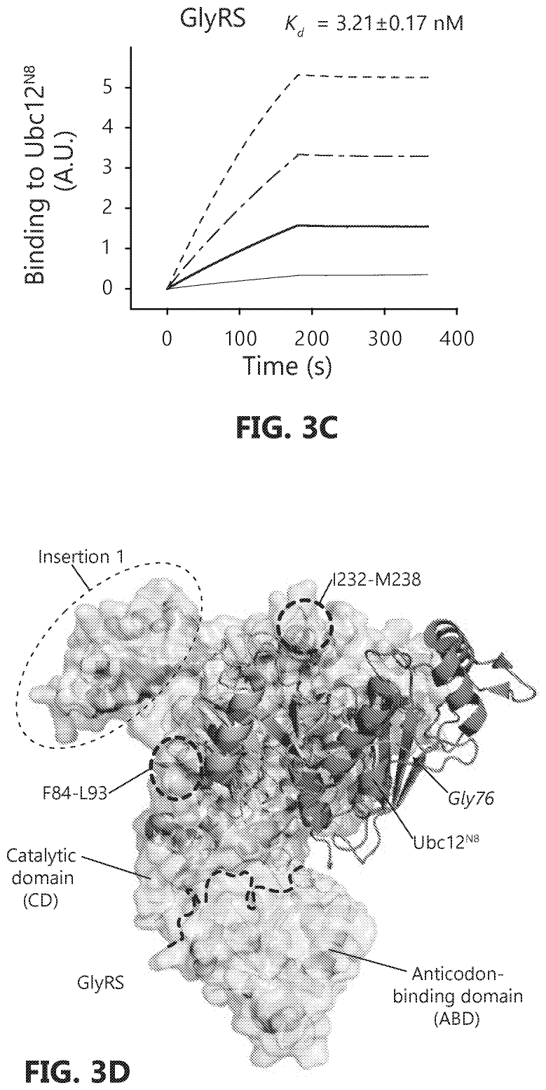

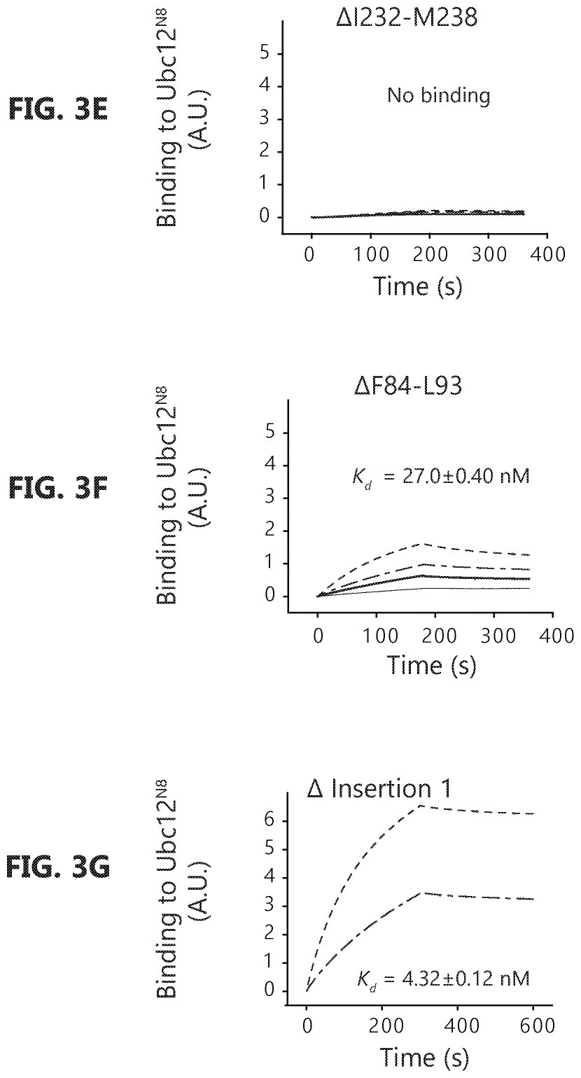

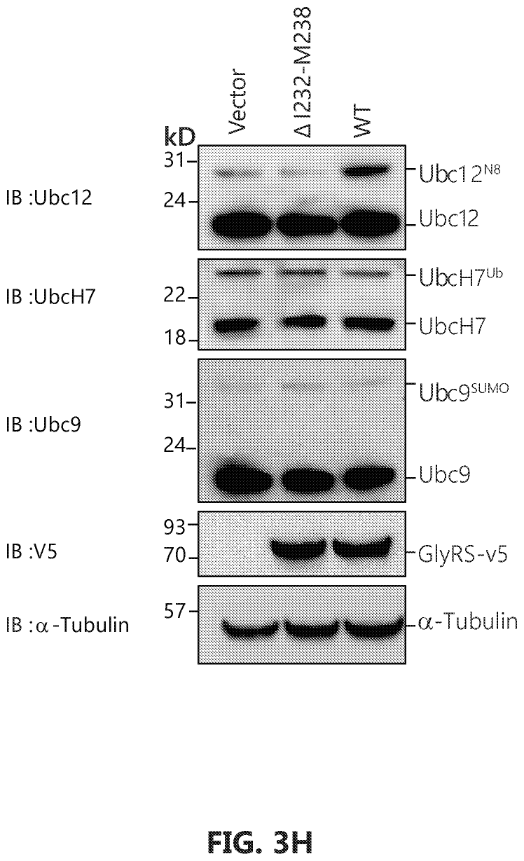

Indeed, GlyRS can bind to Ubc12^{N8} in HEK293 cells as detected by co-immunoprecipitation (FIG. 3a). Moreover, relative to the input, more Ubc12^{N8} than Ubc12 was bound to GlyRS, indicating that GlyRS preferably binds to NEDD8-conjugated Ubc12. To quantify the difference, Ubc12^{N8} was enzymatically made and purified, and its binding affinity to GlyRS was measured using biolayer interferometry. Remarkably, Ubc12^{N8} binds to immobilized GlyRS with a K_{d} of 4.09±0.30 nM, which is 100-fold and 30-fold stronger the K_{d} for Ubc12 alone (488±73 nM) and for NEDD8 alone (126±19 nM), respectively (FIG. 3b). The GlyRS-Ubc12^{N8} interaction was also analyzed reversely by immobilizing Ubc12^{N8} to give a similar K_{d} of 3.21±0.17 nM (FIG. 3c). In the presence of GlyRS, but not BSA, Ubc12^{N8} was significantly more stable than in the absence of GlyRS (FIG. 8c). Therefore, it is possible that GlyRS enhances neddylation by protecting reaction intermediate Ubc12^{N8}.

### Model of GlyRS-Ubc12ⁿ⁸ Complex

Using the crystal structure of human GlyRS16 and of Ubc12N8 (adapted from its complex with substrate cullin and E3), a model was generated for the GlyRS-Ubc12^{N8} interaction using the Patchdock algorithm (http://bioinfo3d.cs.tau.ac.il/PatchDock/). The top solution places Ubc12^{N8} right on top of the dimerization interface of GlyRS, with Ubc12 wedged against motifs 1 and 2 that are conserved for class II tRNA synthetases, and with NEDD8 snuggled in between two β-hairpin loops (F84-L93 and I232-M238) (FIG. 3d). This model is in general consistency with the results obtained from hydrogen-deuterium exchange analysis (FIG. 6 and FIG. 8b). For example, motif 2 and part of motif 1 of GlyRS had decreased levels of deuterium incorporation as a result of Ubc12 binding; the two β-hairpin loops (F84-L93 and I232-M238) also have reduced deuterium incorporation when binding to NEDD8 (FIG. 6). To further validate the model, two deletion mutants of the β-hairpin loops (Δ84-93 and Δ232-238) were created and A84-93 decreased binding of GlyRS to Ubc12^{N8} by 9 fold (K_{d}=27.0±0.40 nM) and A232-238 abolished the binding completely (FIG. 3e & f). Seemingly, Δ232-238 not only disrupts the NEDD8-binding site, but also creates potential conformational changes that affect Ubc12 binding. In contrast, deletion of an insertion domain unique to GlyRS (Insertion 1), which shows no involvement in the GlyRS-Ubc12^{N8} interaction according to the model (FIG. 3d), had no effect on the binding (K_{d}=4.32±0.12 nM) (FIG. 3g). Consistent with its loss of binding to Ubc12^{N8}, the A232-238 GlyRS was completely inactive for promoting Ubc12 conjugation (FIG. 3h), suggesting the function of GlyRS in promoting neddylation is tightly linked to its ability to bind and protect Ubc12^{N8}.

GlyRS Captures Ubc12ⁿ⁸ Released from E1

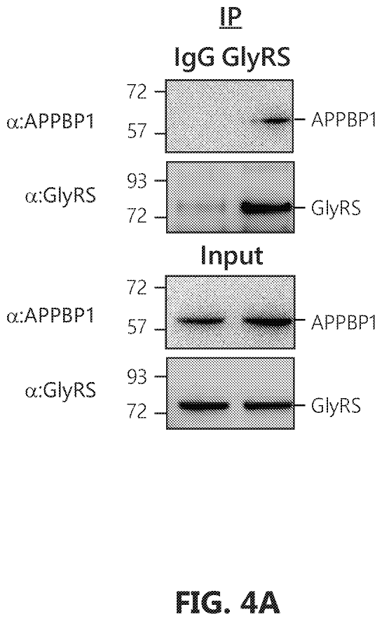

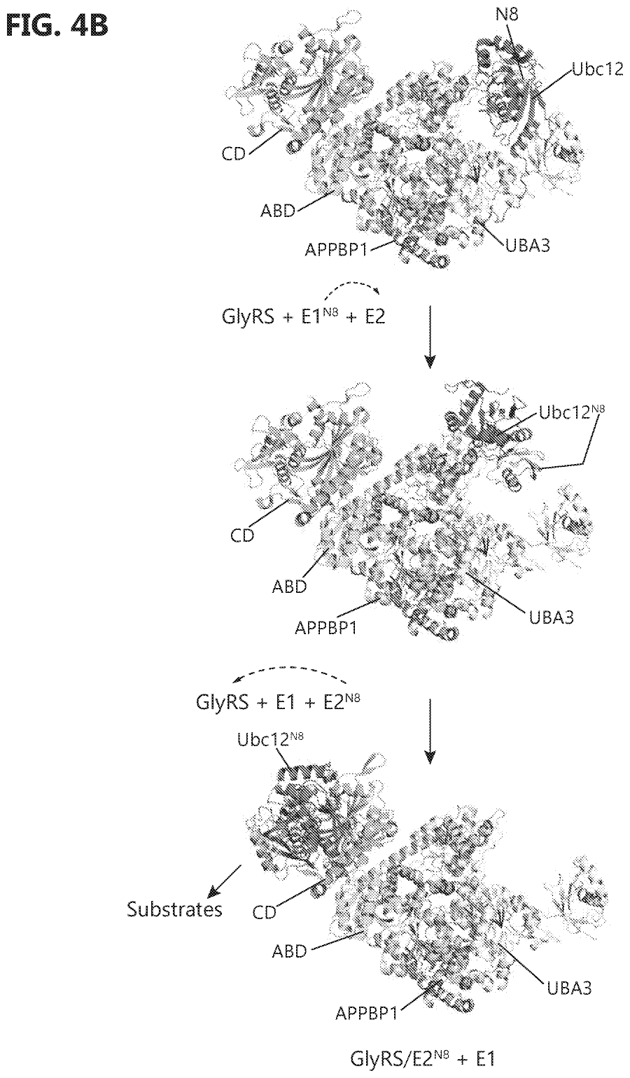

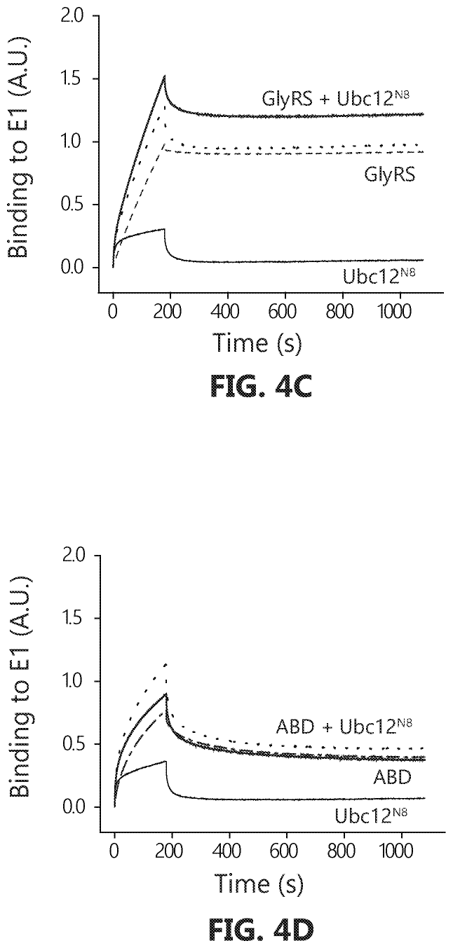

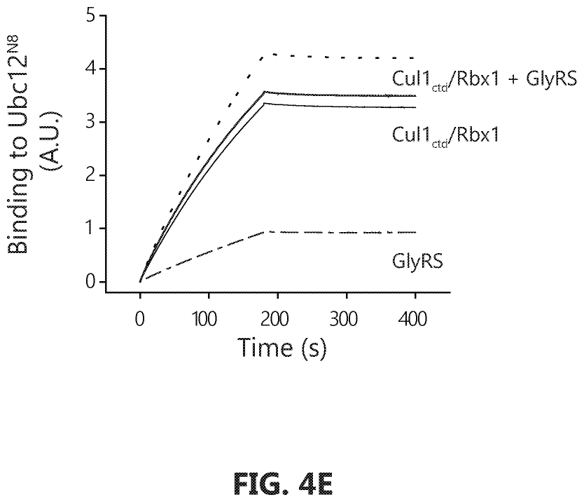

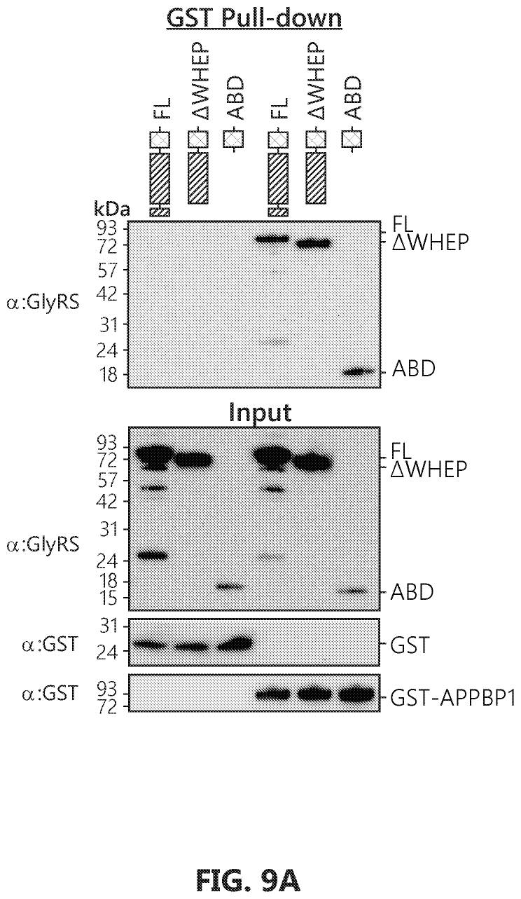

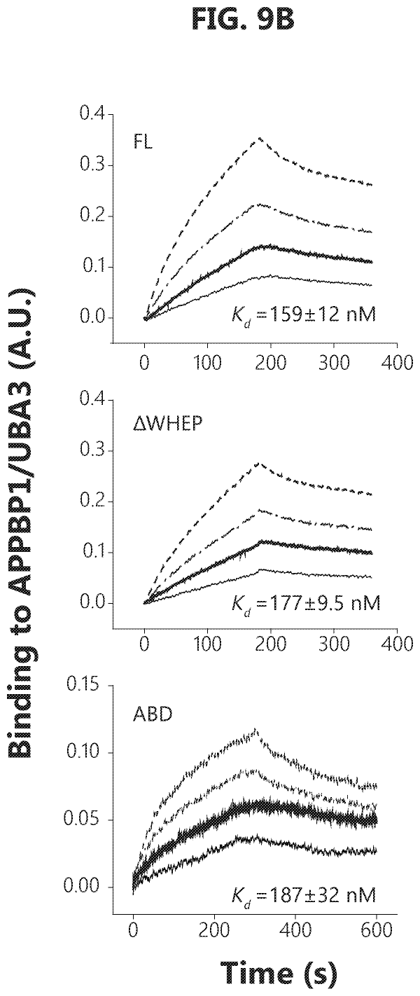

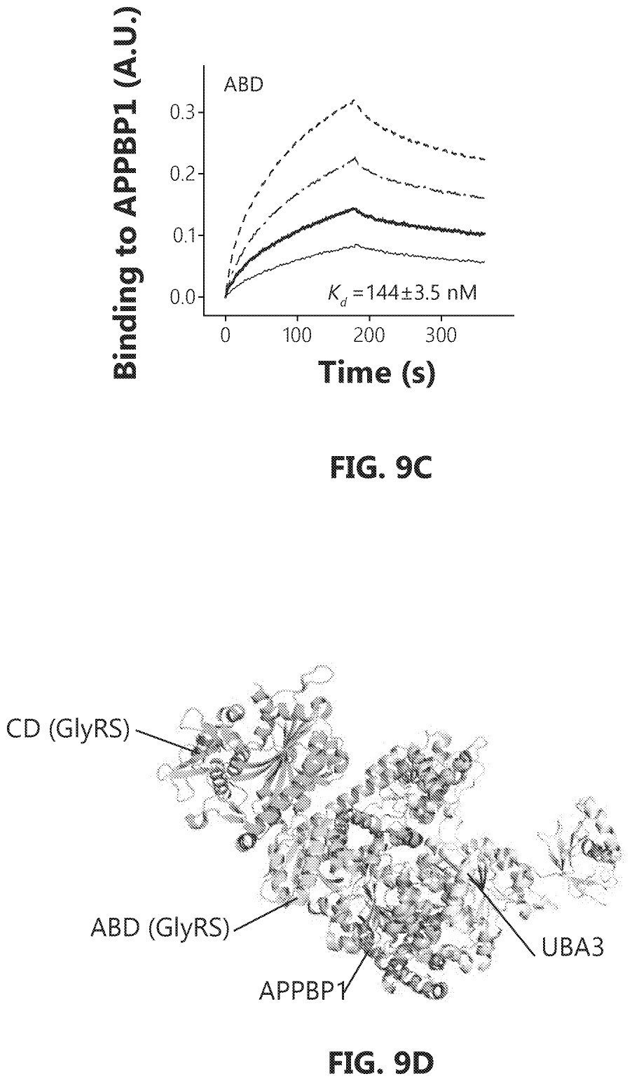

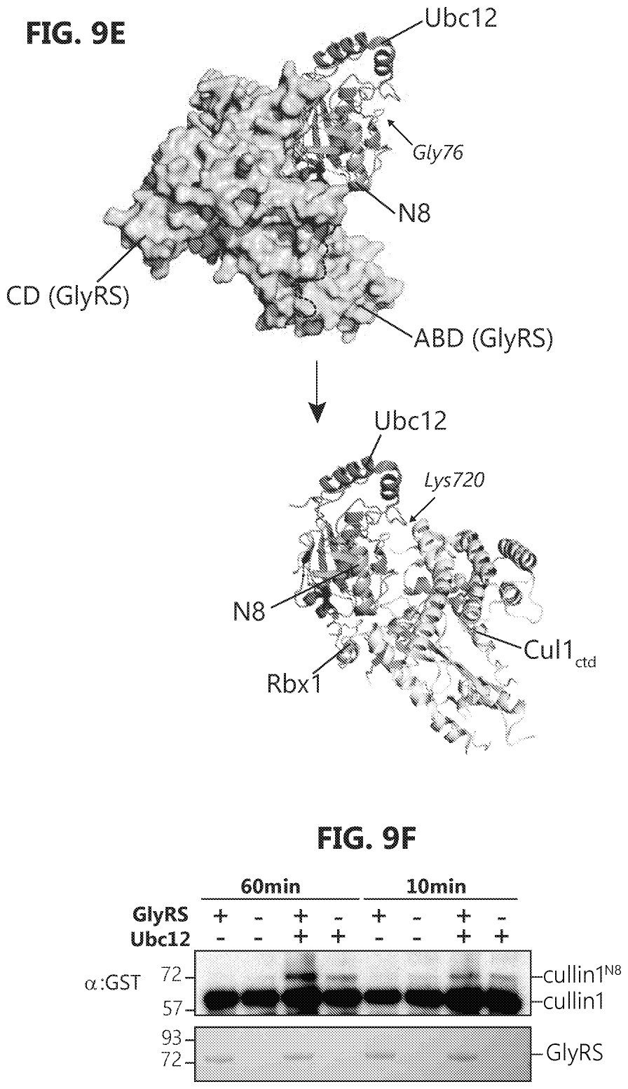

It was found that GlyRS could also bind to the heterodimeric E1 enzyme for neddylation (APPBP1/UBA3) (FIG. 9a & b). However, unlike NEDD8 and Ubc12, E1 binds to the anticodon-binding domain (ABD) of GlyRS, as revealed by both GST-pull down and biolayer interferometry analysis (FIG. 9a,b,c). The APPBP1/UBA3 heterodimer has similar binding affinities to full-length GlyRS (Ka=159±12 nM) and the ABD domain alone (Ka=187±32 nM) (FIG. 9b). Furthermore, similar binding affinity was determined for APPBP1 subunit alone with the ABD of GlyRS (Ka=144±3.5 nM) (FIG. 9c), suggesting that the APPBP1 subunit of E1 is responsible for the GlyRS interaction. The APPBP1-GlyRS interaction was further validated by co-immunoprecipitation in HEK293 cells between endogenous proteins (FIG. 4a). A modeling study through Patchdock also suggested that the ABD of GlyRS interacts with the APPBP1 subunit of the E1 (FIG. 9d).

The fact that GlyRS uses different domains to interact with E1 and Ubc12^{N8} interaction suggests that GlyRS may be able to simultaneously bind to both E1 and Ubc12^{N8}. However, this is not possible if E1 and Ubc12^{N8} are in complex with each other (FIG. 4b). It is important to note that the E1-Ubc12^{N8} interaction is weak, because once NEDD8 is transferred from E1 to E2 during the enzymatic cascade, the association is weakened through conformational changes to facilitate the release of Ubc12^{N8} for the next event. Therefore, the role of GlyRS might be to capture and protect the conjugated E2 after it is released from the E1 and before it finds a correct E3 and/or substrate. By binding to the APPBP1 subunit of E1 through the ABD domain, GlyRS is in proximity to capture the released Ubc12^{N8} (FIG. 4b).

An experiment was designed to test this concept using biolayer interferometry analysis. E1 was immobilized to detect its interaction with Ubc12^{N8} and GlyRS, separately and simultaneously. The binding of Ubc12^{N8} to E1 was weak and the disassociation was faster (FIG. 4c). In contrast, the binding of Ubc12^{N8} to GlyRS was much stronger (FIG. 4c). Interestingly, when both Ubc12^{N8} and GlyRS are present, the overall binding was stronger than the sum of the individual bindings (FIG. 4c), suggesting that a ternary complex of E1-GlyRS-Ubc12^{N8} is formed that prevents Ubc12^{N8} from releasing to the solvent.

The ABD domain of GlyRS was used as a control for this experiment.

Although ABD domain alone interacted with the E1 as strongly as the full length GlyRS (FIG. 9a&b), the absence of the catalytic domain of GlyRS for capturing Ubc12^{N8} would not allow the formation of a ternary complex. Indeed, the ternary complex with ABD domain was not detected (FIG. 4d).

GlyRS does not Interfere with Transferring Ubc12^{N8} to Substrate

Because there is a tight binding between GlyRS and Ubc12^{N8}, it is important to confirm that the binding does not impede Ubc12^{N8} from passing NEDD8 down to its downstream targets such as cullin. Notably, the protection of Ubc12^{N8} by GlyRS does not seem to require burying the thioester bond between Ubc12 and NEDD8. In the model of the GlyRS-Ubc12^{N8} complex (FIG. 3d), the thioester bond between the C-terminal glycine residue Gly76 of NEDD8 and Cys111 of Ubc12 is facing outward rather than buried inside (FIG. 9e). The positioning of this thioester bond is fully compatible with transferring the NEDD8 from Ubc12 to the acceptor residue Lys720 of cullin1 (FIG. 9e). Using cullin1-Rbx1 (RING-box protein 1) as a substrate, it was further tested if cullin1-Rbx1 can bind to Ubc12^{N8} in the presence of GlyRS. It was found that not only cullin1-Rbx1 could bind to Ubc12^{N8} in the presence of GlyRS, the binding seems to release GlyRS from Ubc12^{N8} (FIG. 4e), presumably to allow GlyRS to turnover. Using an in vitro neddylation assay, it was demonstrated that the presence of GlyRS did not decrease but rather increased the neddylation of cullin1 (FIG. 9f).

### GlyRS Promotes Cell Cycle Progression

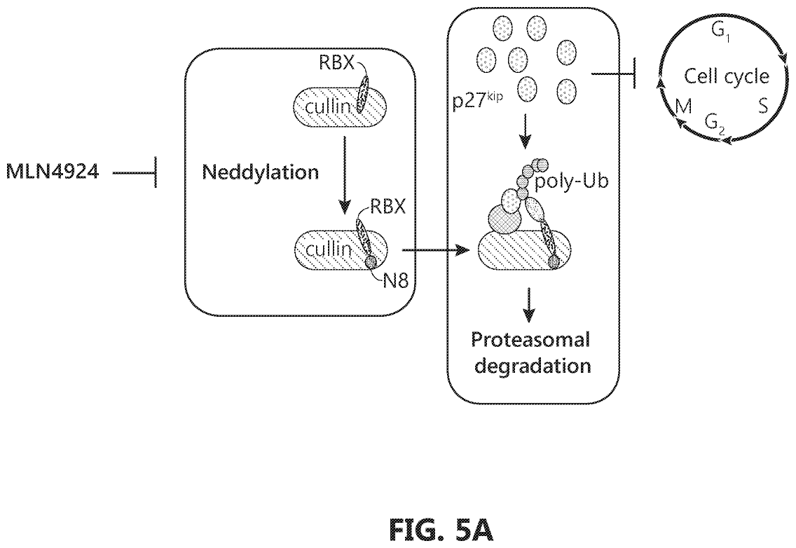

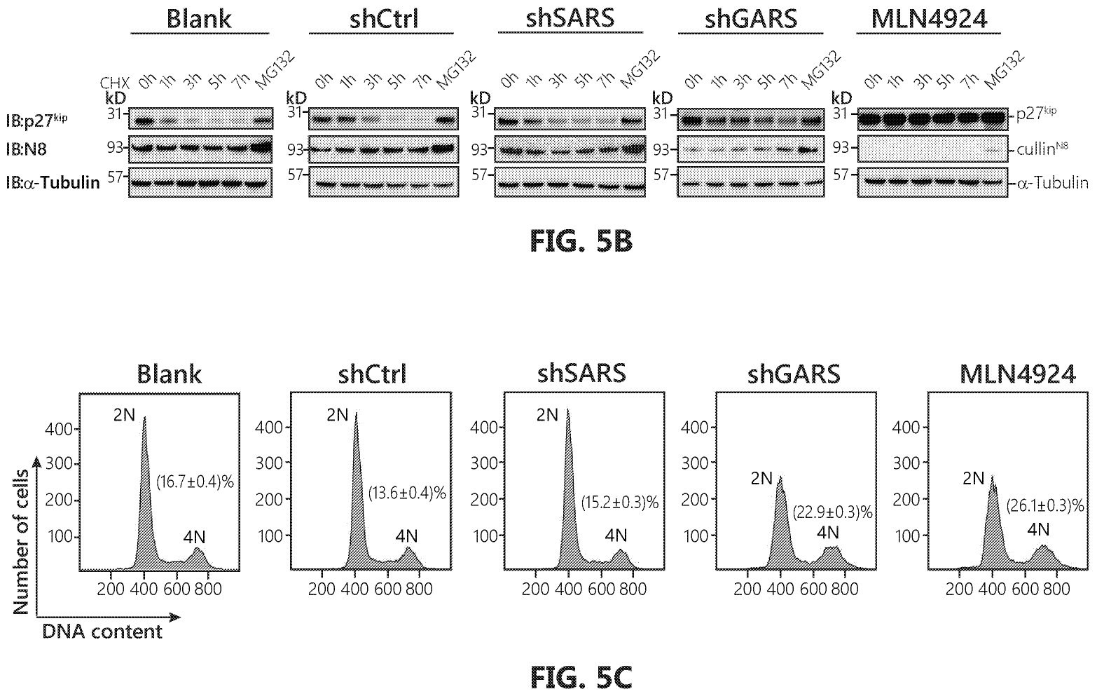

The biological functions of neddylation are best known in the context of cullin proteins as the target. Neddylation of the cullin activates the CRL1(cullin1-RING) ubiquitin ligases and facilitates the degradation of their downstream targets, including cell cycle inhibitor p27^{kip} (FIG. 5a). p27^{kip} induces cell cycle arrest by binding to cyclin-CDK (cyclin-dependent kinase) complexes to inhibit their catalytic activity. Therefore, degradation of p27^{kip} through cullin neddylation promotes cell cycle progression and cell proliferation. Because this is a well-established pathway, p27^{kip} degradation and cell cycle progression were focused on to study the biological role of GlyRS in neddylation.

To evaluate p27^{kip} degradation in cells, cycloheximide, an inhibitor of translation, was used to block new protein synthesis. In control cells (untreated cells or cells transfected with a control shRNA (shCtrl)), the level of p27^{kip} decreased rapidly upon cycloheximide treatment (half-life=2.5 h) (FIG. 5b). (Proteasome inhibitor MG132 was used as a control to confirm the decrease of p27^{kip} level was due to ubiquitin-dependent proteolysis.) However, in cells transfected with an shRNA to knock down GlyRS (shGARS), the half-life of p27^{kip} was significantly extended (˜5 h) (FIG. 5b). And the stabilization of p27^{kip} was concurrent with a decreased level of cullin^{N8} (FIG. 5b). To further confirm that this effect is related to neddylation, a neddylation specific inhibitor MLN4924 was used, which binds to the ATP-binding site of UBA3 to block neddylation²¹. MLN4924 treatment completely abolished cullin neddylation and blocked p27^{kip} degradation (FIG. 5b).

It was expected the stabilization of p27^{kip} would lead to cell cycle arrest. To test flow cytometry cell cycle analysis was used. Indeed, compared with the control cells, HeLa cells transfected with shGARS or treated with MLN4924 showed significantly decreased number of diploid (2N) cells and increased population of tetraploid (4N) cells (FIG. 5c), indicating cell cycle arrest. Therefore, the role of GlyRS in neddylation was linked to cell cycle progression.

The study described in this example provides the first example that a ‘translation factor’ can act in post-translational modification. In fact, multiple components of the translation machinery including tRNA synthetases have been reported as substrates of neddylation, however, there was no indication that they can function directly to influence the post-translational modification pathway. Despite no E1-like activity was found in GlyRS an enhancer role for neddylation has been revealed. This role is achieved through the two essential domains of GlyRS for aminoacylation—the catalytic domain and the anticodon-binding domain (ABD). Results from biochemical and structural analysis support the model, in which GlyRS, by docking on the APPBP1 subunit of E1 through the ABD domain, is in proximity to capture, by the catalytic domain, the NEDD8-conjugated E2 (Ubc12^{N8}) after it is released from the E1 and before it finds the correct E3 and/or substrate to transfer the NEDD8 modifier. The confinement provided by the synthetase would protect the conjugated E2 from random hydrolysis and thereby enhance the overall efficiency of the neddylation pathway. As neddylation is well established for its role in promoting cell cycle progression and proliferation, a synergy may exist between the aminoacylation function of GlyRS to support new protein synthesis and its neddylation enhancer function to stimulate cell proliferation.

The selective NEDD8 E1 inhibitor MLN4924 is currently being tested in several clinical trials for hematological malignancies and solid tumors. As was shown here in FIG. 5, a partial knockdown of GlyRS expression in HeLa cells had a similar effect as MLN4924 in causing cell cycle arrest, suggesting that inhibition of GlyRS may be considered for cancer treatment as well. Importantly, inhibition of GlyRS would not only suppress neddylation but also impede protein synthesis, both of which are undesirable for tumor growth.

According to the online Human Protein Atlas, all components of the neddylation pathway are predominantly localized in the nucleus, consistent with their prominent role in cell cycle regulation. However, many reports suggested that Ubc12, and neddylation substrates are also localized in the cytoplasm, indicative of the existence of other cytoplasmic targets for neddylation. On the other hand, tRNA synthetases are predominantly cytoplasmic proteins for their role in protein synthesis; however, a large repertoire of regulatory functions of tRNA synthetases beyond their enzymatic role in protein synthesis has been reported and many tRNA synthetases are found in the nucleus to carry out important biological functions, such as regulating vascular development, activating p53 signaling, promoting DNA damage response, and regulating gene expression under immunological challenge. Although a nuclear localization signal sequence cannot be readily identified in GlyRS, given the role of GlyRS in neddylation, GlyRS may also reside in the nucleus.

It is interesting to note that often time the regulatory functions of tRNA synthetase are linked to the new domains that were added to the catalytic core during evolution. In GlyRS, this new domain is the WHEP domain, which was added to the synthetase in metazoans. The role of the WHEP domain in regulating neddylation remains to be further characterized, however, based on binding and functional results (FIG. 1e, FIG. 2a and FIG. 9a&b), it appears that the WHEP domain is not directly involved, suggesting that the role of GlyRS in neddylation should not be limited to animals and may exist in all eukaryotes where the modification occurs.

### Example 2

### GlyRS Association with Progression of Multiple Cancer Types

In this example, the association between GlyRS activity and progression of cancer is accessed by analyzing the expression level of GlyRS in multiple cancer patient samples. The results demonstrate that GlyRS is associated with progression of various cancer types, including breast cancer, ovarian cancer, lung cancer, breast duct carcinoma, colorectal adenocarcinoma and lung squamous cell carcinoma.

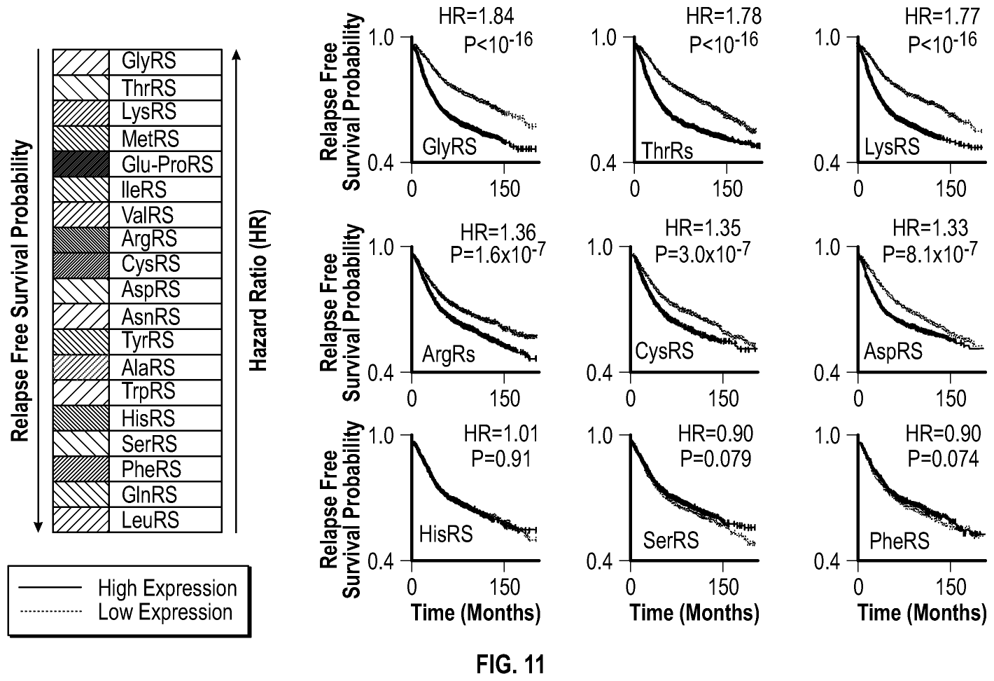

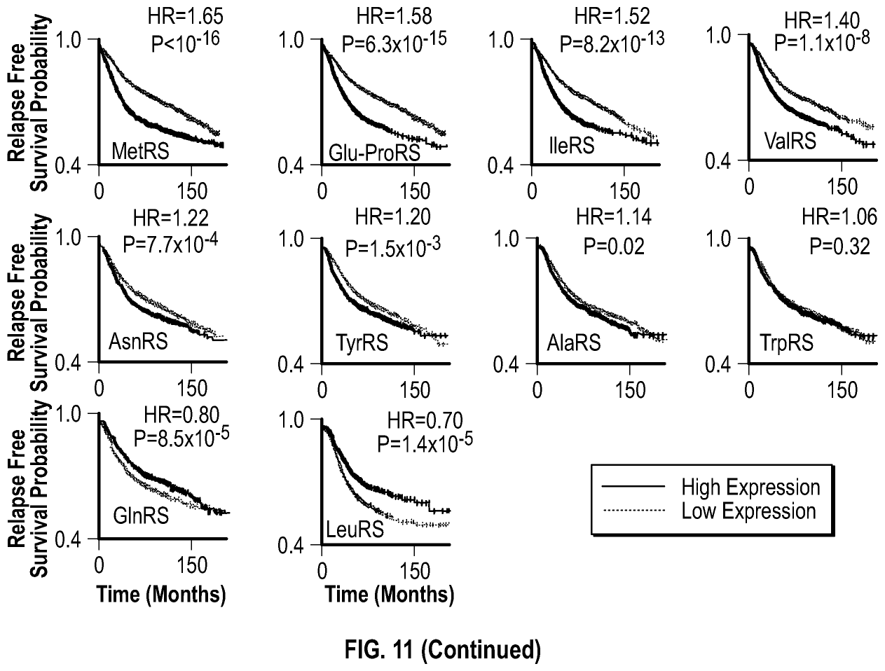

As shown in FIG. 11, high level of GlyRS is associated with rapid breast cancer progression. The Kaplan-Meier plots and hazard ratio (HR) analysis of human tRNA synthetases in breast cancer patients are shown in FIG. 11. Patient samples were divided in halves as low-expression and high-expression sets for each tRNA synthetase in the analysis. n=3,557 patients. P values were calculated with two-sided log-rank tests.

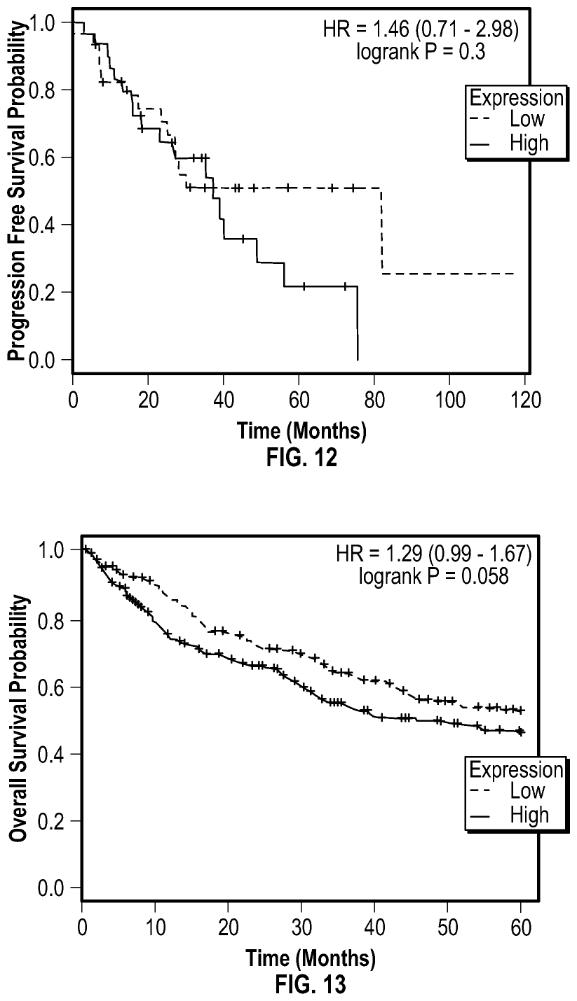

High level of GlyRS was also found to be associated with rapid ovarian cancer progression (see FIG. 12). The Kaplan-Meier plots and hazard ratio (HR) analysis of human GlyRS in stage 2 ovarian cancer patients are shown in FIG. 12. Patient samples were divided in halves as low-expression and high-expression sets for GlyRS in the analysis. n=60 patients. P values were calculated with two-sided log-rank tests.

FIG. 13 shows experimental data demonstrating that high level of GlyRS is associated with rapid lung cancer progression. The Kaplan-Meier plots and hazard ratio (HR) analysis of human GlyRS in lung squamous cell carcinoma patients are shown. Patient samples were divided in halves as low-expression and high-expression sets for GlyRS in the analysis. n=524 patients. P values were calculated with two-sided log-rank tests.

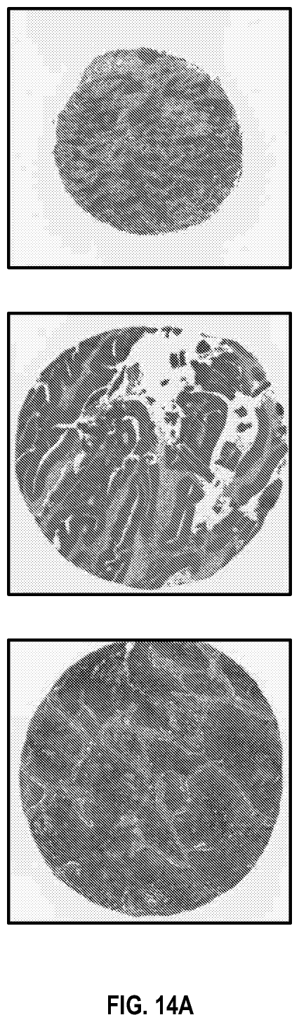

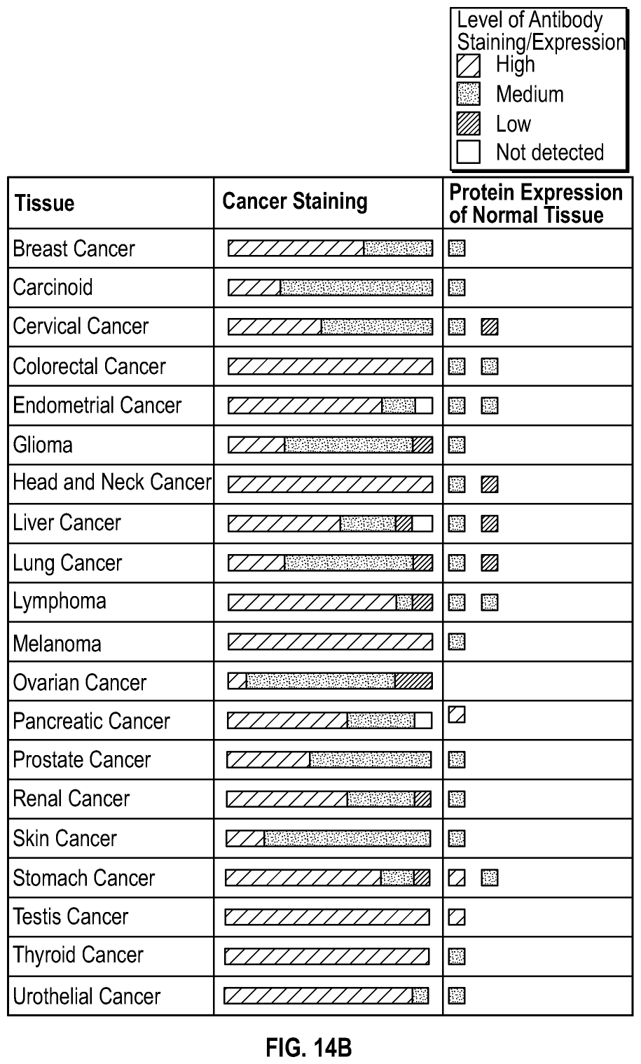

Also shown in FIGS. 14A and 14B, higher level of GlyRS staining in most malignant patient cancer tissue samples. As shown in FIG. 14A, high level staining of GlyRS is observed in patient tissue samples of breast duct carcinoma, colorectal adenocarcinoma and lung squamous cell carcinoma. As shown in FIG. 14B, most malignant patient cancer tissue samples show higher level of GlyRS expression compared to normal tissue.

### Example 3

### GlySA Targets GlyRS with Dual Mechanism

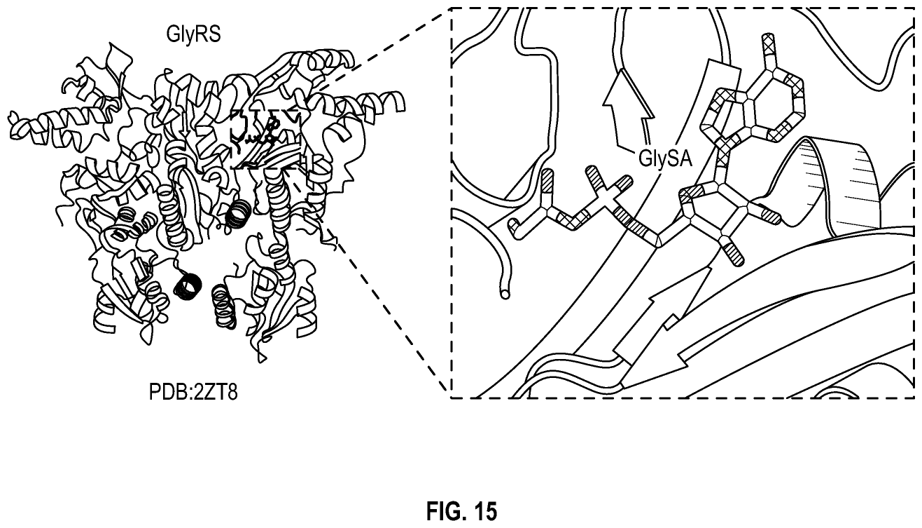

In this example, the mechanism under which GlySA, a GlyRS inhibitor, inhibits GlyRS, was investigated. FIG. 15 shows a non-limiting schematic illustration of GlySA binding to GlyRS active site. GlySA is an analog of Gly-AMP, reaction intermediate of GlyRS. The inhibitory effects of GlySA on GlyRS aminoacylation and neddylation were tested in both cell lines and mice models.

### Biochemical Experiments

Biochemical experiments were performed to study the inhibiting activity of GlySA to GlyRS. Aminoacylation assay was performed using recombinant human GlyRS (200 nM) proteins at room temperature (RT). MLN4924 is a known inhibitor of neddylation currently used in clinical trials for multiple solid and hematopoietic cancers. MLN4924 targets the E1 enzyme (UBA3) of neddylation.

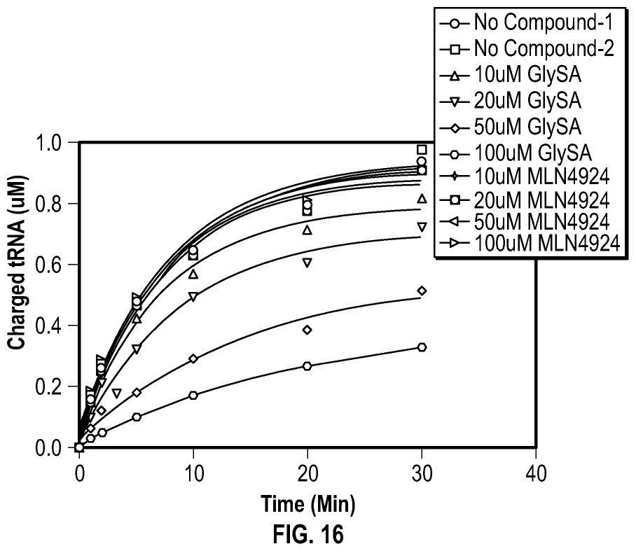

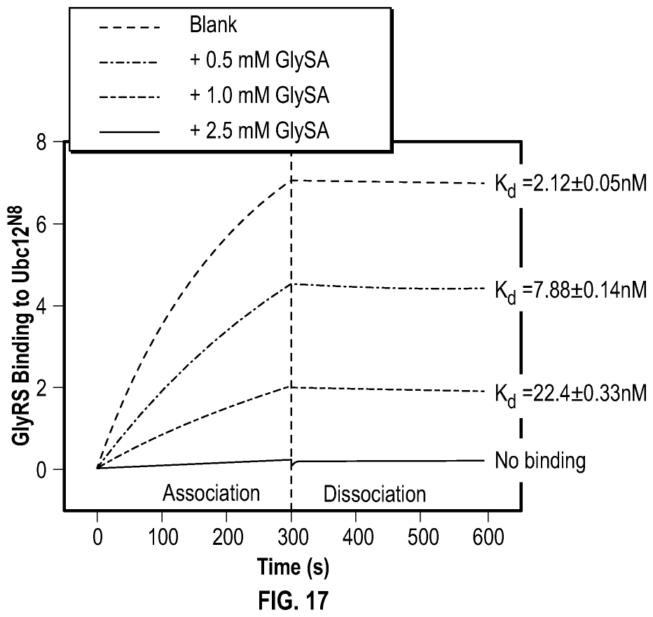

As shown in FIG. 16, GlySA (but not MLN4924) inhibits GlyRS aminoacylation. As shown in FIG. 17, GlySA decreases GlyRS binding to activated NEDD8 E2 (Ubc12^{N8}). The interactions of GlyRS to that of Ubc12^{N8} were compared in the presence of DMSO or GlySA at 30° C. by biolayer interferometry (Octet).

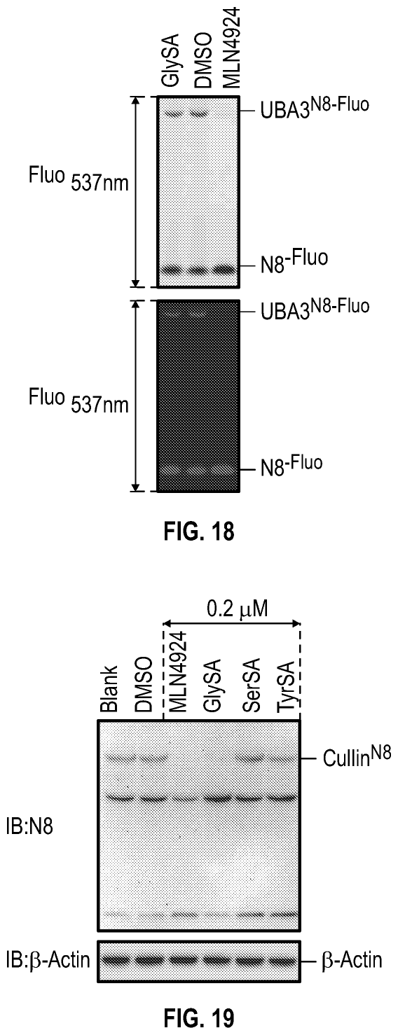

In vitro NEDD8 activation assay was performed with recombinant human APPBP1-UBA3 (2.7 μM) protein and fluorescein-labeled NEDD8 proteins in the reaction buffer at 37° C. for 1 hour. The concentration of GlySA and MLN4924 was 300 μM. FIG. 18 shows that unlike MLN4924, GlySA does not affect NEDD8 E1 (UBA3) activation.

### Cell Experiments

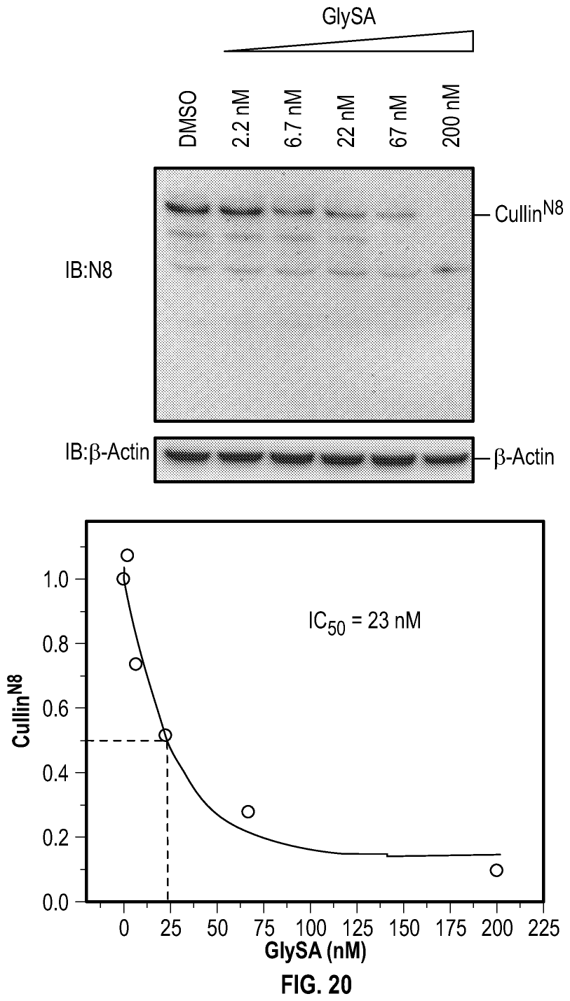

Cell experiments were also performed to study the inhibiting activity of GlySA to GlyRS. MDA-MB-231 cells at 80% confluence were treated with compounds for overnight and then the cells were harvested and lysed with the acid lysis buffer and subjected to non-reducing SDS-PAGE. SerSA and TyrSA are analogs of Ser-AMP and Tyr-AMP, reaction intermediate of SerRS and TyrRS, respectively. It was found that GlySA, but not SerSA, TyrSA, inhibits neddylation in MDA-MB-231 cells (FIG. 19). Concentration range of GlySA in inhibiting neddylation in MDA-MB-231 cells were also evaluated and the results are shown in FIG. 20.

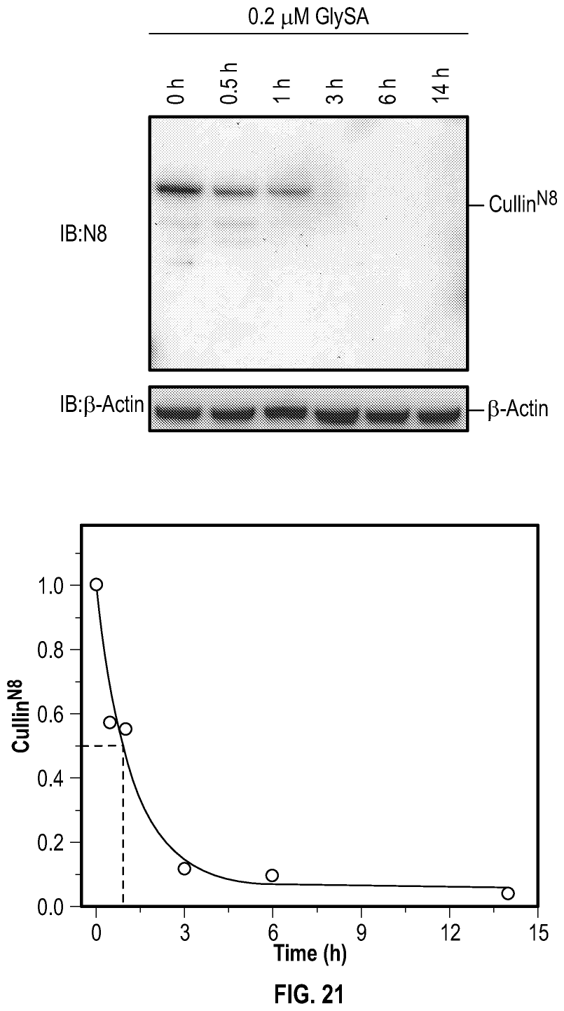

MDA-MB-231 cells at 80% confluence were treated with 200 nM GlySA and then the cells were harvested at different time points and lysed with the acid lysis buffer and subjected to non-reducing SDS-PAGE. FIG. 21 shows experimental data on time course of GlySA in inhibiting neddylation in MDA-MB-231 cells.

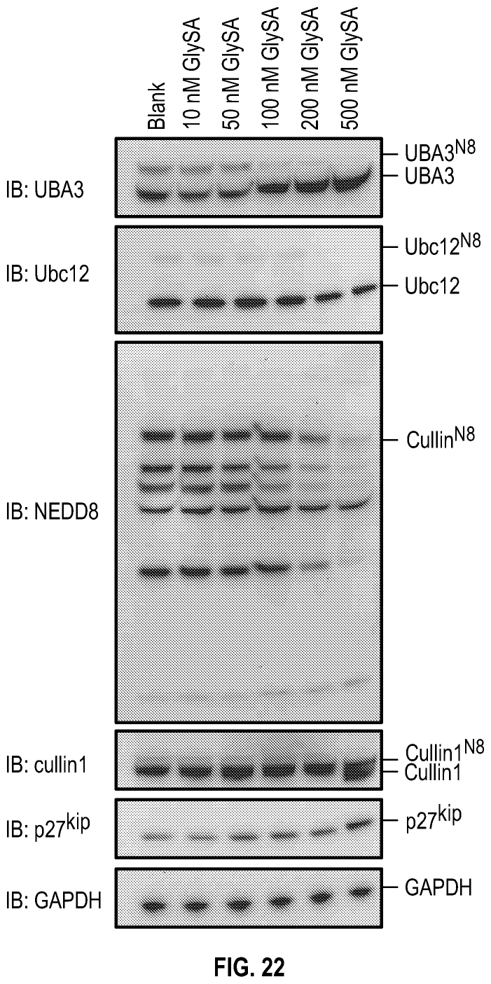

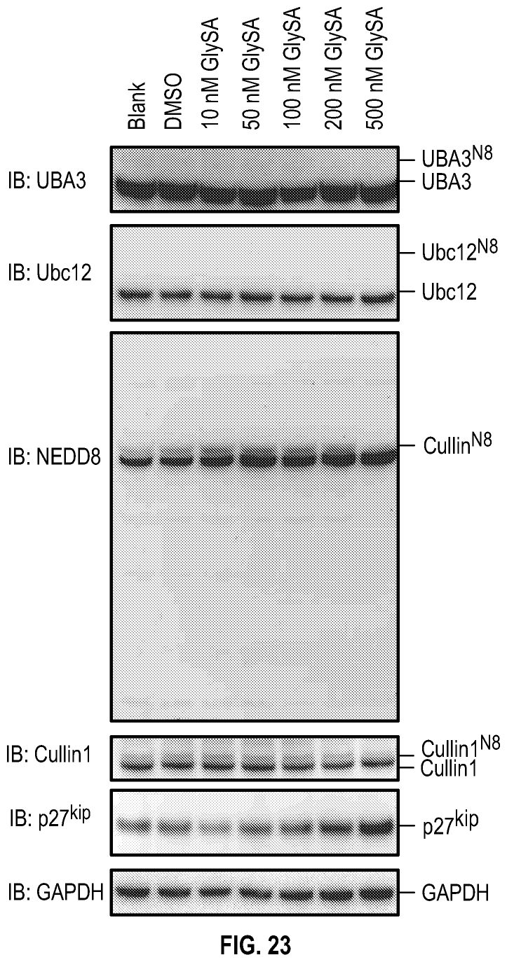

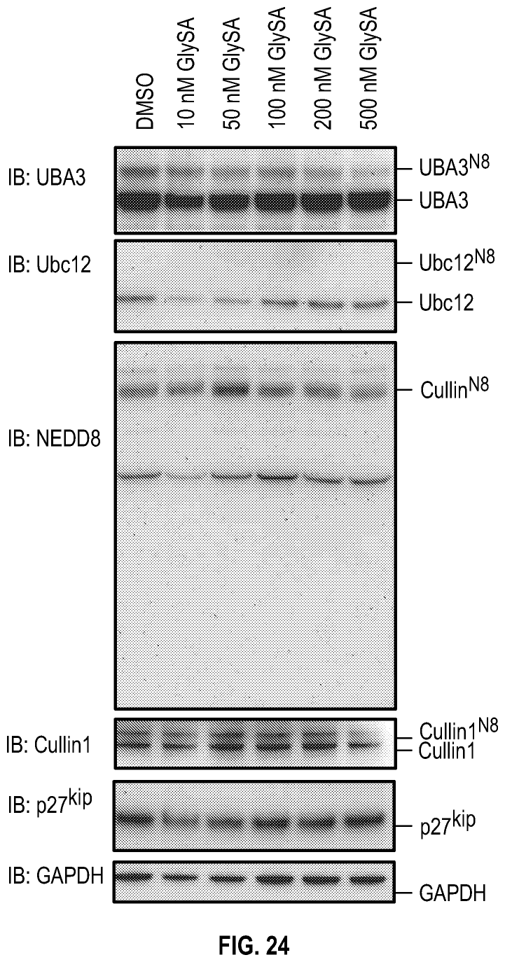

The inhibitory effect of GlySA on key components and substrates of the neddylation pathways were studied using various cancer cell lines. MDA-MB-231 cells at 80% confluence were treated with compounds for overnight and then the cells were harvested and lysed with the acid lysis buffer and subjected to non-reducing SDS-PAGE. FIG. 22 shows GlySA's effect on key components and substrates of the neddylation pathway in MDA-MB-231 cells. MDA-MB-468 cells at 80% confluence were treated with compounds for overnight and then the cells were harvested and lysed with the acid lysis buffer and subjected to non-reducing SDS-PAGE. FIG. 23 shows experimental data on GlySA effect on key components and substrates of neddylation in MDA-MB-468 cells. MCF7 cells at 80% confluence were also treated with compounds for overnight and then the cells were harvested and lysed with the acid lysis buffer and subjected to non-reducing SDS-PAGE. FIG. 24 shows experimental data on GlySA effect on key components and substrates of neddylation in MCF7 cells.

### NCI 60 Cell One-Dose Screen

### General Description:

As of early 2007 all compounds submitted to the NCI 60 Cell screen are tested initially at a single high dose (10-5 M) in the full NCI 60 cell panel. Only compounds which satisfy pre-determined threshold inhibition criteria in a minimum number of cell lines will progress to the full 5-dose assay. The threshold inhibition criteria for progression to the 5-dose screen was selected to efficiently capture compounds with anti-proliferative activity based on careful analysis of historical DTP screening data. The threshold criteria may be updated as additional data becomes available.

### Interpretation of One-Dose Data:

The One-dose data will be reported as a mean graph of the percent growth of treated cells and will be similar in appearance to mean graphs from the 5-dose assay. The number reported for the One-dose assay is growth relative to the no-drug control, and relative to the time zero number of cells. This allows detection of both growth inhibition (values between 0 and 100) and lethality (values less than 0). This is the same as for the 5-dose assay, described below. For example, a value of 100 means no growth inhibition. A value of 40 would mean 60% growth inhibition. A value of 0 means no net growth over the course of the experiment. A value of −40 would mean 40% lethality. A value of −100 means all cells are dead. Information from the One-dose mean graph is available for COMPARE analysis.

### NCI 60 Cell Five-Dose Screen

### General Description:

Compounds which exhibit significant growth inhibition in the One-Dose Screen are evaluated against the 60 cell panel at five concentration levels.

The human tumor cell lines of the cancer screening panel grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO2, 95% air and 100% relative humidity for 24 h prior to addition of experimental drugs.

After 24 hours, two plates of each cell line fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO2, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti>=Tz

[(Ti−Tz)/Tz]×00 for concentrations for which Ti<Tz.

Three dose response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

The general procedures of NCI 60 Cell One-Dose Screen and NCI 60 Cell Five-Dose Screen were used to evaluate the inhibitory effect of GlySA on the growth of various cancer cell lines. The results are shown in Table 2 (one-dose screen) and Table 3 (five-dose screen) below.

TABLE 2
List of cancer cell lines sorted according to the GlySA growth inhibition effect.

| Panel name | Cell name | Growth inhibition count (%) |
|---|---|---|
| Renal Cancer | A498 | −58.80134788 |
| Non-Small Cell Lung Cancer | NCI-H226 | −54.57056308 |
| Non-Small Cell Lung Cancer | NCI-H522 | −51.58866995 |
| Melanoma | MDA-MB-435 | −49.81617647 |
| Melanoma | LOX IMVI | −45.56709265 |
| CNS Cancer | U251 | −39.33823529 |
| Renal Cancer | RXF 393 | −36.59850484 |
| Melanoma | SK-MEL-2 | −34.37833511 |
| Breast Cancer | MDA-MB-468 | −34.302595 |
| CNS Cancer | SF-295 | −33.68333333 |
| Non-Small Cell Lung Cancer | HOP-62 | −32.11206897 |
| Prostate Cancer | DU-145 | −31.72348485 |
| Melanoma | M14 | −23.10222672 |
| Ovarian Cancer | OVCAR-4 | −22.10026212 |
| Ovarian Cancer | OVCAR-8 | −11.91763727 |
| CNS Cancer | SF-539 | −9.360885276 |
| Leukemia | HL-60(TB) | −9.284776903 |
| Renal Cancer | 786-0 | −7.021837349 |
| CNS Cancer | SNB-75 | −6.717877095 |
| CNS Cancer | SNB-19 | −6.057336621 |
| Melanoma | MALME-3M | −2.822580645 |
| Non-Small Cell Lung Cancer | NCI-H322M | −1.738410596 |
| Melanoma | SK-MEL-5 | −0.3413371 |
| Ovarian Cancer | OVCAR-5 | 0.291560931 |
| Colon Cancer | KM12 | 0.734685773 |
| Renal Cancer | SN12C | 1.029729281 |
| Leukemia | MOLT-4 | 1.145822073 |
| Ovarian Cancer | NCI/ADR-RES | 2.072589755 |
| Breast Cancer | MDA-MB-231/ATCC | 2.607853364 |
| Non-Small Cell Lung Cancer | NCI-H460 | 3.350093377 |
| Colon Cancer | SW-620 | 3.712061898 |
| CNS Cancer | SF-268 | 3.776035626 |
| Renal Cancer | CAKI-1 | 5.110152835 |
| Non-Small Cell Lung Cancer | NCI-H23 | 5.695418902 |
| Colon Cancer | HT29 | 6.002098636 |
| Renal Cancer | UO-31 | 6.083971978 |
| Ovarian Cancer | SK-OV-3 | 6.281150543 |
| Colon Cancer | HCT-116 | 6.566078895 |
| Leukemia | K-562 | 7.376219586 |
| Breast Cancer | T-47D | 7.655948339 |
| Breast Cancer | MCF7 | 8.271239384 |
| Melanoma | SK-MEL-28 | 8.357448779 |
| Prostate Cancer | PC-3 | 9.662392044 |
| Non-Small Cell Lung Cancer | A549/ATCC | 9.701463415 |
| Leukemia | SR | 9.925740754 |
| Ovarian Cancer | OVCAR-3 | 10.04947249 |
| Colon Cancer | HCT-15 | 11.41530732 |
| Ovarian Cancer | IGROV1 | 11.70363841 |
| Renal Cancer | ACHN | 11.7925978 |
| Breast Cancer | HS 578T | 16.07429649 |
| Non-Small Cell Lung Cancer | HOP-92 | 17.26373251 |
| Leukemia | RPMI-8226 | 18.03788521 |
| Breast Cancer | BT-549 | 21.05943912 |
| Leukemia | CCRF-CEM | 21.67622168 |
| Melanoma | UACC-257 | 27.43273074 |
| Melanoma | UACC-62 | 30.70756508 |
| Colon Cancer | HCC-2998 | 41.35051734 |
| Non-Small Cell Lung Cancer | EKVX | 44.07083614 |
| Colon Cancer | COLO 205 | 48.50851164 |

TABLE 3
List of cancer cell lines sorted according to the GlySA (LC₅₀/GI₅₀).

| Panel name | Cell name | GI₅₀ (μM) | LC₅₀ (μM) | LC₅₀/GI₅₀ |
|---|---|---|---|---|
| Non-Small Cell Lung Cancer | HOP-62 | 0.372 | >100 | >269.2 |
| CNS Cancer | SF-268 | 0.501 | >100 | >199.5 |
| Renal Cancer | SN12C | 0.562 | 95.50 | 169.8 |
| Prostate Cancer | DU-145 | 0.692 | >100 | >144.5 |
| Ovarian Cancer | OVCAR-5 | 0.741 | >100 | >134.9 |
| Leukemia | SR | 0.933 | >100 | >107.2 |
| Ovarian Cancer | OVCAR-4 | 0.389 | 23.99 | 61.7 |
| Breast Cancer | T-47D | 2.344 | >100 | >42.7 |
| Ovarian Cancer | OVCAR-8 | 2.455 | >100 | >40.7 |
| Ovarian Cancer | NCI/ADR-RES | 2.512 | >100 | >39.8 |
| Non-Small Cell Lung Cancer | EKVX | 2.570 | >100 | >38.9 |
| Non-Small Cell Lung Cancer | NCI-H23 | 2.570 | >100 | >38.9 |
| Leukemia | MOLT-4 | 2.630 | >100 | >38.0 |
| Leukemia | HL-60(TB) | 2.754 | >100 | >36.3 |
| Breast Cancer | MCF7 | 2.818 | >100 | >35.5 |
| Breast Cancer | HS 578T | 2.884 | >100 | >34.7 |
| Breast Cancer | MDA-MB-231/ATCC | 0.525 | 18.20 | 34.7 |
| Colon Cancer | HT29 | 2.951 | >100 | >33.9 |
| Colon Cancer | HCT-116 | 3.162 | >100 | >31.6 |
| Colon Cancer | KM12 | 3.236 | >100 | >30.9 |
| Non-Small Cell Lung Cancer | NCI-H460 | 3.236 | >100 | >30.9 |
| Leukemia | RPMI-8226 | 3.311 | >100 | >30.2 |
| Colon Cancer | COLO 205 | 2.291 | 64.57 | 28.2 |
| Colon Cancer | HCT-15 | 2.951 | 81.28 | 27.5 |
| Leukemia | CCRF-CEM | 3.715 | >100 | >26.9 |
| Leukemia | K-562 | 3.715 | >100 | >26.9 |
| Colon Cancer | SW-620 | 3.715 | >100 | >26.9 |
| Prostate Cancer | PC-3 | 2.630 | 56.23 | 21.4 |
| CNS Cancer | SNB-19 | 0.380 | 6.92 | 18.2 |
| Ovarian Cancer | IGROV1 | 3.020 | 47.86 | 15.8 |
| Non-Small Cell Lung Cancer | NCI-H322M | 1.514 | 22.91 | 15.1 |
| Ovarian Cancer | SK-OV-3 | 2.884 | 37.15 | 12.9 |
| Renal Cancer | ACHN | 2.884 | 33.88 | 11.7 |
| Breast Cancer | BT-549 | 2.344 | 25.12 | 10.7 |
| CNS Cancer | U251 | 0.550 | 5.75 | 10.5 |
| Breast Cancer | MDA-MB-468 | 0.741 | 7.59 | 10.2 |
| Non-Small Cell Lung Cancer | HOP-92 | 2.951 | 27.54 | 9.3 |
| Renal Cancer | TK-10 | 2.754 | 23.44 | 8.5 |
| CNS Cancer | SNB-75 | 1.148 | 9.55 | 8.3 |
| Non-Small Cell Lung Cancer | A549/ATCC | 2.344 | 19.05 | 8.1 |
| CNS Cancer | SF-295 | 0.871 | 6.92 | 7.9 |
| CNS Cancer | SF-539 | 1.445 | 8.91 | 6.2 |
| Ovarian Cancer | OVCAR-3 | 1.995 | 10.00 | 5.0 |
| Non-Small Cell Lung Cancer | NCI-H522 | 1.738 | 8.51 | 4.9 |
| Colon Cancer | HCC-2998 | 2.042 | 9.33 | 4.6 |
| Melanoma | SK-MEL-28 | 1.950 | 8.91 | 4.6 |
| Melanoma | SK-MEL-2 | 2.239 | 10.00 | 4.5 |
| Melanoma | LOX IMVI | 1.698 | 7.41 | 4.4 |
| Melanoma | UACC-62 | 1.862 | 7.76 | 4.2 |
| Renal Cancer | A498 | 1.585 | 6.46 | 4.1 |
| Melanoma | MALME-3M | 1.905 | 7.76 | 4.1 |
| Melanoma | M14 | 1.820 | 7.24 | 4.0 |
| Renal Cancer | RXF 393 | 2.138 | 8.32 | 3.9 |
| Renal Cancer | 786-0 | 1.778 | 6.76 | 3.8 |
| Melanoma | MDA-MB-435 | 1.820 | 6.61 | 3.6 |
| Renal Cancer | UO-31 | 1.698 | 6.17 | 3.6 |
| Non-Small Cell Lung Cancer | NCI-H226 | 1.995 | 7.08 | 3.5 |
| Melanoma | SK-MEL-5 | 1.862 | 6.03 | 3.2 |

### Mice Experiments

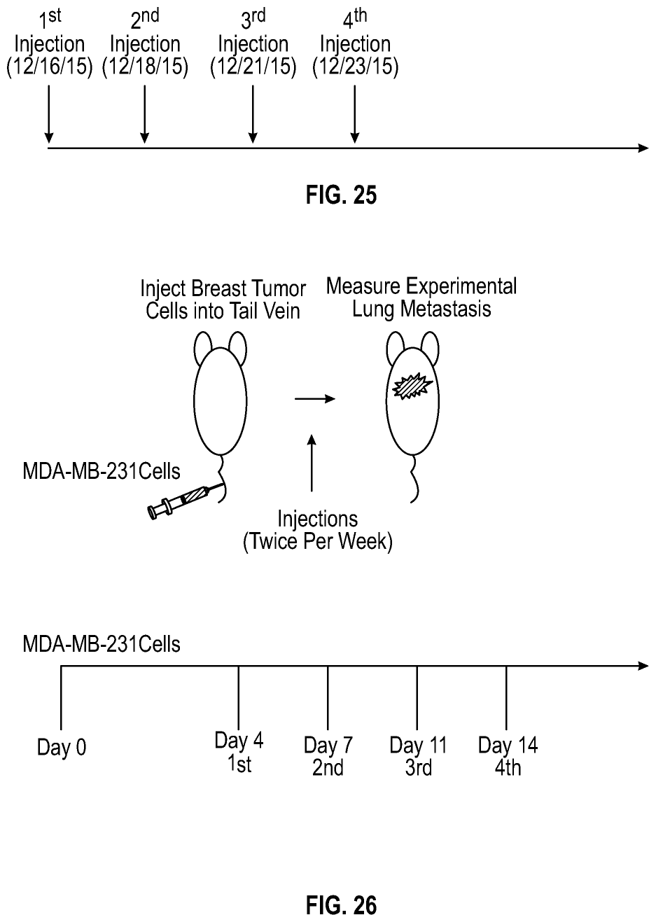

Animal experiments were also performed to study the use of GlySA to treat cancer. A maximum tolerant dosage assay of GlySA was conducted. GlySA (DMSO stock solution diluted by saline) were administrated to three month old female BALB CJ mice via tail vein injections. Mice after four injections were evaluated and all were alive. The GlySA concentration tested were 0.4 mg/kg (10 μM), 2.0 mg/kg (50 μM), 4.0 mg/kg (100 μM). N=3 for each group. A schematic illustration of the assay is shown in FIG. 25.

FIG. 26 shows a schematic illustration of lung metastasis assay methods conducted. 1×10⁵ MDA-MB-231 cells were injected via tail vein to NOD.Cg-Prkdc^{scid} Il2rg mice. Then mice were separated into 3 groups. Group A: vehicle alone (PBS with 1% DMSO), group B: GlySA (4 mg/kg; 100 μM), and group C: MLN4924 GlySA (4.4 mg/kg; 100 μM) were administrated via tail vein injections twice per week. N=10 for each group.

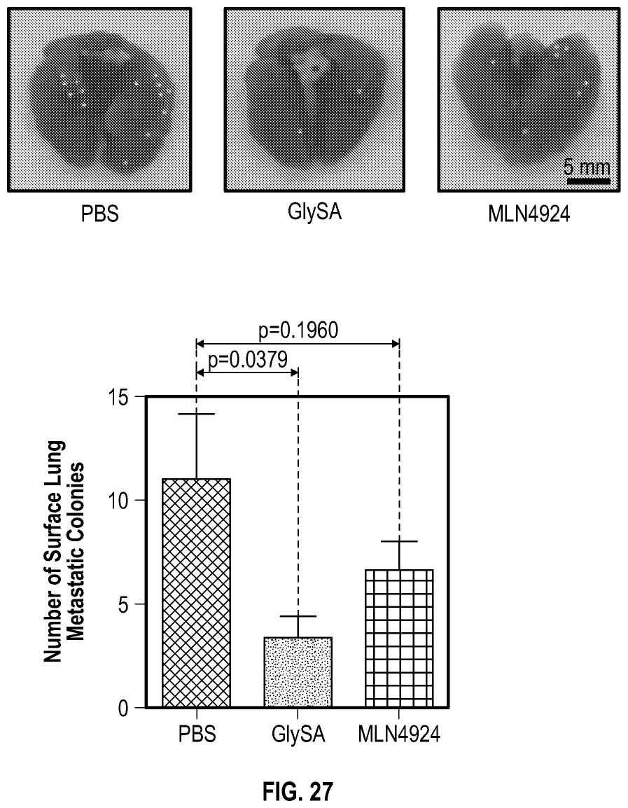

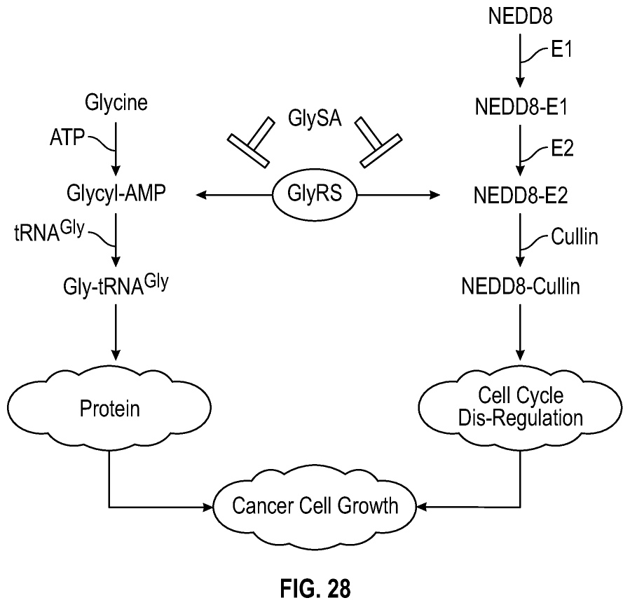

It was found that GlySA treatment reduces lung metastasis in mice (FIG. 27). Top panel of FIG. 27 shows mice lungs 14 days after tumor cells (MDA-MB-231) injection. White dots show the surface tumor colonies. Bottom panel of FIG. 27 shows numbers of lung metastasis colonies are analyzed by two tails unpaired T test. The error bars represent SEM (n=8-10).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

1. A method of reducing neddylation in a cell, the method comprises:
contacting a cell with a composition comprising a Glycyl-tRNA synthetase (GlyRS) inhibitor,
wherein the level of neddylation is decreased in the cell.

2. (canceled)

3. (canceled)

4. (canceled)

5. (canceled)

6. (canceled)

7. (canceled)

8. The method claim 1, wherein the GlyRS inhibitor is an inhibitor for a human GlyRS gene product.

9. The method of claim 8, wherein the human GlyRS gene product comprises an amino acid sequence having at least 90% identity to the amino acid sequence set forth in SEQ ID NO: 2.

10. (canceled)

11. The method of claim 1, wherein the GlyRS inhibitor is a protein, a nucleic acid, a small molecule compound, or a combination thereof.

12. The method of claim 11, wherein the GlyRS inhibitor is an RNA molecule capable of silencing the expression of a GlyRS gene.

13. The method of claim 11, wherein the GlyRS inhibitor is an RNA molecule that binds to an mRNA encoded by a GlyRS gene.

14. (canceled)

15. The method of claim 13, wherein the GlyRS gene is the human GARS gene.

16. The method of claim 15, wherein the human GARS gene comprises a nucleotide sequence having at least 90% sequence identity to the nucleotide sequence set forth in SEQ ID NO: 1.

17. The method of claim 16, wherein the GlyRS inhibitor is a short-hairpin RNA (shRNA) comprising a nucleotide sequence having at least 90% sequence identity to the nucleic acid sequence of SEQ ID NOs:3-35.

18. The method of claim 1, wherein the GlyRS inhibitor is GlySA or a derivative thereof.

19. The method of claim 1, wherein the GlyRS inhibitor interferes with the binding between the GlyRS protein and NEDD8 in the cell.

20. The method of claim 1, wherein the GlyRS inhibitor interferes with the binding between the GlyRS protein and Ubc12 in the cell.

21. The method of claim 1, wherein the GlyRS inhibitor interferes with the binding between the GlyRS protein and NEDD8-conjugated Ubc12 in the cell.

22. The method of claim 21, wherein the GlyRS inhibitor binds to one or more of amino acids 84-93 and 232-238 and amino acids Arg277, Glu279, Val289, Glu296, Ile404, and Glu552 of SEQ ID NO: 2.

23. (canceled)

24. (canceled)

25. (canceled)

26. The method of claim 1, wherein the GlyRS inhibitor decreases the amount of NEDD8-conjugated Ubc12 in the cell, wherein the GlyRS inhibitor decreases Ubc12 activity in the cell, wherein the GlyRS inhibitor increases Ubc12 degradation in the cell, wherein the GlyRS inhibitor interferes with the binding between a GlyRS protein and heterodimeric E1 enzyme for neddylation (APPBP1/UBA3) in the cell, or wherein the GlyRS inhibitor decreases neddylation of a cullin protein in the cell.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A pharmaceutical composition comprising a Glycyl-tRNA synthetase (GlyRS) inhibitor and a pharmaceutically acceptable excipient,
wherein the pharmaceutical composition comprises an isolated double-stranded ribonucleic acid (dsRNA) molecule that inhibits expression of a Glycyl-tRNA synthetase (GlyRS) gene, wherein a first strand of the dsRNA is substantially identical to at least 19 consecutive nucleotides of the GlyRS gene, and a second strand of the dsRNA is substantially complementary to the first strand, or an isolated single stranded oligonucleotide that is complementary to a portion of a Glycyl-tRNA synthetase (GlyRS) gene of at least 10 consecutive nucleotides.

55. (canceled)

56. (canceled)

57. (canceled)

58. The method of claim 54, wherein the GlyRS inhibitor inhibits GlyRS functions in aminoacylation and neddylation.

59. The method of claim 54, wherein the GlyRS inhibitor does not significantly inhibit GlyRS function in aminoacylation.

60. (canceled)

61. (canceled)

62. (canceled)

63. (canceled)

64. (canceled)

65. (canceled)

66. (canceled)

67. (canceled)

68. (canceled)

69. (canceled)

70. A method of treating or ameliorating cancer in a subject, comprising:
administering a therapeutically effective amount of a pharmaceutical composition comprising a Glycyl-tRNA synthetase (GlyRS) inhibitor to a subject in need thereof.

71. (canceled)

72. (canceled)

73. (canceled)

74. (canceled)

75. (canceled)

76. (canceled)

77. (canceled)

78. (canceled)

79. The method of claim 70, wherein the GlyRS inhibitor does not significantly inhibit GlyRS function in aminoacylation, and wherein the GlyRS inhibitor is an inhibitor for a human GlyRS.

80. (canceled)
